(19) United States
(12) Patent Application Publication
(10) Pub. No.: US 2025/0362596 A1
(43) Pub. Date: Nov. 27, 2025

(54) RADIATION-SENSITIVE COMPOSITION, METHOD FOR FORMING PATTERN, AND RADIATION-SENSITIVE ACID GENERATOR

(71) Applicant: JSR CORPORATION, Tokyo (JP)

(72) Inventors: Ryuichi NEMOTO, Tokyo (JP); Michihiro MITA, Tokyo (JP); Atsuto NISHII, Tokyo (JP); Hajime INAMI, Tokyo (JP)

(73) Assignee: JSR CORPORATION, Tokyo (JP)

(21) Appl. No.: 19/293,284

(22) Filed: Aug. 7, 2025

Related U.S. Application Data
(63) Continuation-in-part of application No. PCT/JP2024/007065, filed on Feb. 27, 2024.

(30) Foreign Application Priority Data
Feb. 28, 2023 (JP) 2023-029806

(51) Int. Cl. G03F 7/004; C07C 381/12; G03F 7/00; G03F 7/038; G03F 7/039; G03F 7/32

(52) U.S. Cl. CPC G03F 7/0045; C07C 381/12; G03F 7/0046; G03F 7/0382; G03F 7/0388; G03F 7/0397; G03F 7/322; G03F 7/70033

(57) ABSTRACT

A radiation-sensitive composition includes: an onium salt compound represented by formula (1); a polymer including a structural unit which includes an acid-dissociable group; and a solvent. In the formula (1), W is an organic group having 3 to 40 carbon atoms and having at least one cyclic structure; L is a (r+1)-valent linking group, and r is an integer of 1 to 3; when r is 1, p and q are each independently an integer of 1 to 3, and when r is 2 or 3, each of a plurality of p's and a plurality of q's are each independently an integer of 0 to 3, provided that when r is 2 or 3, at least one of a plurality of p's is 1 or more and at least one of a plurality of q's is 1 or more; M⁺ is a monovalent onium cation.

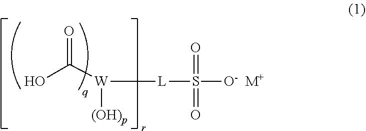

### CROSS-REFERENCE TO RELATED APPLICATIONS

The present application is a continuation-in-part application of International Patent Application No. PCT/JP2024/007065 filed Feb. 27, 2024, which claims priority to Japanese Patent Application No. 2023-029806 filed Feb. 28, 2023. The contents of these applications are incorporated herein by reference in their entirety.

### BACKGROUND OF THE DISCLOSURE

### Technical Field

The present disclosure relates to a radiation-sensitive composition, a method for forming a pattern and a radiation-sensitive acid generator.

### Background Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a resin into an alkaline or organic developer between an exposed part and a non-exposed part.

In the photolithography technique, the micronization of the pattern is promoted by using a short-wavelength radioactive ray such as an ArF excimer laser or by using an immersion exposure method (liquid immersion lithography) in which exposure is performed in a state in which a space between a lens of an exposure apparatus and a resist film is filled with a liquid medium. As a next-generation technology, lithography using shorter wavelength radiation such as electron beams, X-rays and EUV (extreme ultraviolet rays) is also being considered.

To form a finer resist pattern in the formation of a circuit of a semiconductor device by a photolithography technique, various studies have been conducted on a photoacid generator, which is one of main components of a resist composition (see, for example, JP-A-2020-75910 and JP-B2-5083528).

### SUMMARY

According to an aspect of the present disclosure, a radiation-sensitive composition includes: an onium salt compound represented by formula (1); a polymer including a structural unit which includes an acid-dissociable group; and a solvent.

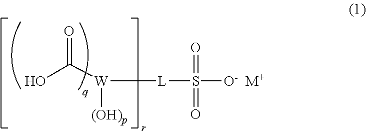

In the formula (1), W is an organic group having 3 to 40 carbon atoms and having at least one cyclic structure; L is a (r+1)-valent linking group, and r is an integer of 1 to 3; when r is 1, p and q are each independently an integer of 1 to 3, and when r is 2 or 3, each of a plurality of p's and a plurality of q's are each independently an integer of 0 to 3, provided that when r is 2 or 3, at least one of a plurality of p's is 1 or more and at least one of a plurality of q's is 1 or more; M⁺ is a monovalent onium cation.

According to another aspect of the present disclosure, a method for forming a pattern, includes: applying the above-described radiation-sensitive composition directly or indirectly to a substrate to form a resist film; exposing the resist film to light; and developing the exposed resist film. According to a further aspect of the present disclosure, a radiation-sensitive acid generator is represented by formula (1),

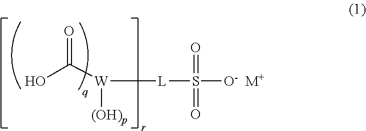

In the formula (1), W is an organic group having 3 to 40 carbon atoms and having at least one cyclic structure; L is a (r+1)-valent linking group, and r is an integer of 1 to 3; when r is 1, p and q are each independently an integer of 1 to 3, and when r is 2 or 3, each of a plurality of p's and a plurality of q's are each independently an integer of 0 to 3, provided that when r is 2 or 3, at least one of a plurality of p's is 1 or more and at least one of a plurality of q's is 1 or more; M⁺ is a monovalent onium cation.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

The resist composition is required to have various resist performances such as sensitivity, line width roughness (LWR), which indicates variations in a line width and a line width of a resist pattern, pattern rectangularity, which indicates rectangularity of a sectional shape of a resist pattern, development defect performance, exposure latitude (EL), critical dimension uniformity (CDU), which indicates uniformity of a hole diameter, and pattern circularity, which indicates roundness of a hole shape.

That is, the present disclosure relates, in an embodiment, to a radiation-sensitive composition comprising:
an onium salt compound represented by formula (1) below (hereinafter, also referred to as “onium salt compound (1)”);
a polymer containing a structural unit having an acid-dissociable group; and
a solvent,

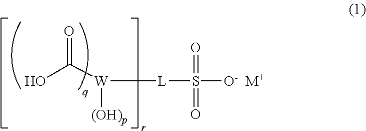

in the formula (1), W is an organic group having 3 to 40 carbon atoms and having at least one cyclic structure; L is a (r+1)-valent linking group, and r is an integer of 1 to 3; when r is 1, both p and q are integers of 1 to 3, and when r is 2 or 3, each of a plurality of p's and a plurality of q's is an integer of 0 to 3, provided that when r is 2 or 3, at least one of a plurality of p's is 1 or more and at least one of a plurality of q's is 1 or more; M⁺ is a monovalent onium cation.

Since the radiation-sensitive composition contains the onium salt compound (1), a resist film exhibiting excellent sensitivity, LWR, pattern rectangularity, development defect performance, EL, CDU, and pattern circularity can be formed. The reason for this is not bound by any theory, but can be expected as follows.

The anion of the onium salt compound (1) has a carboxy group and a hydroxy group, and these groups interact with the polymer in the composition, so that the diffusion length of a generated acid can be appropriately shortened, and LWR and EL can be improved. In addition, it is presumed that since the anion of the onium salt compound (1) has a carboxy group and a hydroxy group, solubility in a developer is greatly improved, and insoluble components are reduced, so that development defects can be more efficiently suppressed, and given various resist performances can be exhibited.

The present disclosure relates, in another embodiment, to a method for forming a pattern, comprising:
applying the radiation-sensitive composition directly or indirectly to a substrate to form a resist film;
exposing the resist film to light; and
developing the exposed resist film.

In the method for forming a pattern, a high-quality resist pattern can be efficiently formed because of the use of the radiation-sensitive composition capable of forming a resist film excellent in sensitivity, LWR, pattern rectangularity, development defect performance, EL, CDU performance, and pattern circularity.

The present disclosure relates, in still another embodiment, to a radiation-sensitive acid generator represented by formula (1):

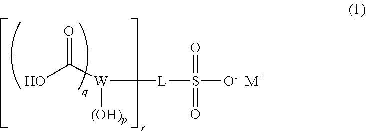

in the formula (1), W is an organic group having 3 to 40 carbon atoms and having at least one cyclic structure; L is a (r+1)-valent linking group, and r is an integer of 1 to 3; when r is 1, both p and q are integers of 1 to 3, and when r is 2 or 3, each of a plurality of p's and a plurality of q's is an integer of 0 to 3, provided that when r is 2 or 3, at least one of a plurality of p's is 1 or more and at least one of a plurality of q's is 1 or more; M⁺ is a monovalent onium cation.

Since the radiation-sensitive acid generator contains the onium salt compound (1) having the above specific structure, good sensitivity, LWR, pattern rectangularity, development defect performance, EL, CDU, and pattern circularity can be imparted to a resist film obtained when the radiation-sensitive acid generator is used in a radiation-sensitive composition.

Hereinbelow, embodiments of the present disclosure will be described in detail, but the present disclosure is not limited to these embodiments. Combinations of suitable embodiments are also preferable.

### <Radiation-Sensitive Composition>

The radiation-sensitive composition (hereinafter also simply referred to as “composition”) according to the present embodiment includes an onium salt compound (1), a polymer containing a structural unit having an acid-dissociable group, and a solvent. The composition may further contain other optional components as long as the effects of the present invention are not impaired. Owing to the inclusion of specific onium salt compound (1) as radiation-sensitive acid generators in a radiation-sensitive composition, the radiation-sensitive composition can impart sensitivity, LWR, pattern rectangularity, development defect performance, EL, CDU, and pattern circularity at high levels to a resist film of the radiation-sensitive composition.

### (Onium Salt Compound (1))

The onium salt compound (1) is represented by the above formula (1), and functions as a radiation-sensitive acid generator, which generates an acid upon irradiation with radiation. Depending on the structure of the onium salt compound (1), the onium salt compound (1) also functions as a radiation-sensitive strong acid generator, and can also function as an acid diffusion controlling agent, which generates an acid having a pka higher than that of the acid to be generated from the radiation-sensitive strong acid generator upon irradiation with radiation. In the present disclosure, it is preferable to use the onium salt compound (1) as a radiation-sensitive strong acid generator from the viewpoint of development defect performance. The onium salt compound (1) as a radiation-sensitive strong acid generator will be described below.

The organic group having 3 to 40 carbon atoms, containing at least one cyclic structure, and represented by W is not particularly limited, and may be either a group containing only a cyclic structure or a group containing a cyclic structure and a chain structure in combination. The cyclic structure may be any of a monocyclic structure, a polycyclic structure, or a combination thereof. In addition, the cyclic structure may be any of an alicyclic structure, an aromatic ring structure, a heterocyclic structure, or a combination thereof. In the case of a combination, the combination may be a structure in which cyclic structures may be bonded by a chain structure, or two or more cyclic structures may form a condensed cyclic structure or a bridged cyclic structure. The number of the cyclic structures in the organic group is just required to be 1, or may be 2 or more. There may intervene a divalent hetero atom-containing group between adjacent carbon atoms forming the backbone of the cyclic structure or chain structure, and a hydrogen atom on a carbon atom in the cyclic structure or chain structure may be replaced with another substituent.

Examples of alicyclic structures include a monovalent alicyclic hydrocarbon group having 3 to 20 carbon atoms. Examples of the monovalent alicyclic hydrocarbon group having 3 to 20 carbon atoms include monovalent monocyclic or polycyclic saturated hydrocarbon groups and monocyclic or polycyclic unsaturated hydrocarbon groups. As the monocyclic saturated hydrocarbon groups, a cyclopentyl group, a cyclohexyl group, a cycloheptyl group, and a cyclooctyl group are preferable. As the polycyclic cycloalkyl group, bridged alicyclic hydrocarbon groups such as a norbornyl group, an adamantyl group, a tricyclodecyl group, and a tetracyclododecyl group are preferable. Examples of the monocyclic unsaturated hydrocarbon group include monocyclic cycloalkenyl groups such as a cyclopropenyl group, a cyclobutenyl group, a cyclopentenyl group, and a cyclohexenyl group. Examples of the polycyclic unsaturated hydrocarbon group include polycyclic cycloalkenyl groups such as a norbornenyl group, a tricyclodecenyl group, and a tetracyclododecenyl group. The bridged alicyclic hydrocarbon group refers to a polycyclic alicyclic hydrocarbon group in which two carbon atoms that constitute an alicyclic ring and are not adjacent to each other are bonded by a linking group containing one or more carbon atoms.

Examples of aromatic ring structures include a monovalent aromatic hydrocarbon group having 6 to 20 carbon atoms. Examples of the monovalent aromatic hydrocarbon group having 6 to 20 carbon atoms include: aryl groups such as a phenyl group, a tolyl group, a xylyl group, a naphthyl group, and an anthryl group; and aralkyl groups such as a benzyl group, a phenethyl group, and a naphthylmethyl group.

Examples of the heterocyclic structure include a group obtained by removing one hydrogen atom from an aromatic heterocyclic structure and a group obtained by removing one hydrogen atom from an alicyclic heterocyclic structure. A 5-membered aromatic structure having aromaticity due to introduction of a hetero atom is also included in the heterocyclic structure. Examples of the hetero atom include an oxygen atom, a nitrogen atom, and a sulfur atom.

Examples of the aromatic heterocyclic structure include:
oxygen atom-containing aromatic heterocyclic structures such as furan, pyran, benzofuran, and benzopyran;
nitrogen atom-containing aromatic heterocyclic structures such as pyrrole, imidazole, pyridine, pyrimidine, pyrazine, indole, quinoline, isoquinoline, acridine, phenazine, and carbazole;
sulfur atom-containing aromatic heterocyclic structures such as thiophene; and
aromatic heterocyclic structures containing a plurality of hetero atoms such as thiazole, benzothiazole, thiazine, and oxazine.

Examples of the alicyclic heterocyclic structure include:
oxygen atom-containing alicyclic heterocyclic structures such as oxirane, tetrahydrofuran, tetrahydropyran, dioxolane, and dioxane;
nitrogen atom-containing alicyclic heterocyclic structures such as aziridine, pyrrolidine, piperidine, and piperazine;
sulfur atom-containing alicyclic heterocyclic structures such as thietane, thiolane, and thiane; and
alicyclic heterocyclic structures containing a plurality of hetero atoms such as morpholine, 1,2-oxathiolane, and 1,3-oxathiolane;
a lactone structure, a cyclic carbonate structure, and a sultone structure.

The heterocyclic structures include a lactone structure, a cyclic carbonate structure, a sultone structure, a cyclic acetal, or a combination thereof.

Examples of the chain structure include a monovalent chain organic group having 1 to 30 carbon atoms. The monovalent chain organic group having 1 to 30 carbon atoms is not particularly limited as long as a chain structure is possessed. Examples of the chain structure include a monovalent chain hydrocarbon group having 1 to 30 carbon atoms, which may be saturated or unsaturated, linear or branched, a group obtained by substituting some or all of hydrogen atoms contained in the chain hydrocarbon group with a substituent, a group containing a divalent hetero atom-containing group in a carbon-carbon bond of these groups, or a combination thereof.

Examples of the monovalent chain hydrocarbon group having 1 to 30 carbon atoms include a linear or branched saturated hydrocarbon group having 1 to 30 carbon atoms and a linear or branched unsaturated hydrocarbon group having 1 to 30 carbon atoms. Examples of the linear or branched saturated hydrocarbon group having 1 to 30 carbon atoms include alkyl groups such as a methyl group, an ethyl group, a n-propyl group, an i-propyl group, a n-butyl group, a 2-methylpropyl group, a 1-methylpropyl group, a t-butyl group, a n-pentyl group, an isopentyl group, a neopentyl group, a n-hexyl group, an i-hexyl group, a n-heptyl group, and an i-heptyl group. Examples of the linear or branched unsaturated hydrocarbon group having 1 to 30 carbon atoms include alkenyl groups such as an ethenyl group, a propenyl group, and a butenyl group; and alkynyl groups such as an ethynyl group, a propynyl group, and a butynyl group.

Examples of the substituent that substitutes some or all of the hydrogen atoms of the chain hydrocarbon group include halogen atoms such as a fluorine atom, a chlorine atom, a bromine atom, and an iodine atom; a hydroxy group; a carboxy group; a cyano group; a nitro group; an amino group; an aldehyde group; a thiol group; and an oxo group (═O).

As the divalent hetero atom-containing group in the group containing the divalent hetero atom-containing group in a carbon-carbon bond of the chain hydrocarbon group, —CO—, —C(═O)O—, —CS—, —O—, —S—, —SO₂—, and —NR″—, and a combination of two or more thereof can be suitably used. R″ represents a hydrogen atom or a monovalent hydrocarbon group having 1 to 5 carbon atoms. When the chain hydrocarbon group has the divalent hetero atom-containing groups, the number of the divalent hetero atom-containing groups is preferably 1, 2, or 3, and more preferably 1 or 2.

The bonding sites of the carboxy groups and the hydroxy groups bonded to W in the formula (1) are not particularly limited, and these groups may be bonded anywhere on the structure represented by W. Preferably, the carboxy groups and the hydroxy groups are bonded directly or indirectly to the same cyclic structure or different cyclic structures. More preferably, the carboxy groups and the hydroxy groups are bonded directly to the same cyclic structure or different cyclic structures. Still more preferably, the carboxy groups and the hydroxy groups are bonded directly to the same cyclic structure, and at least one hydroxy group is bonded to a carbon atom adjacent to a carbon atom to which a carboxy group is bonded.

As an embodiment in which a carboxy group and a hydroxy group are bonded directly or bonded indirectly to the same cyclic structure, the partial structure “—W(OH)ₚ(COOH)_{q}” in the formula (1) preferably contains one or more groups selected from the group consisting of groups represented by the following formulas (W-1) to (W-5).

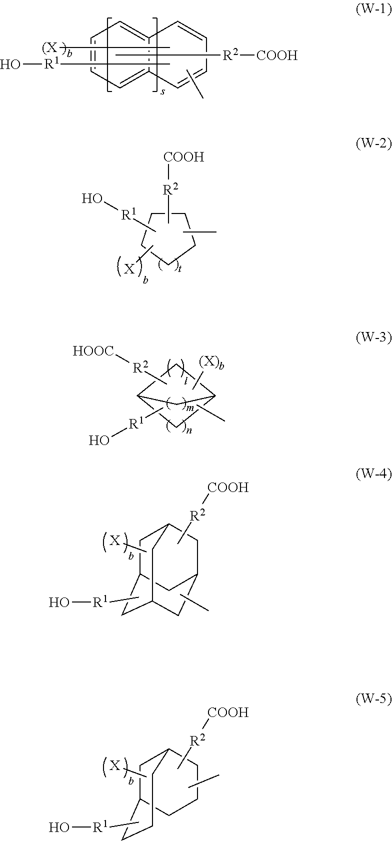

wherein, s is an integer of 0 to 2 and t is an integer of 1 to 3; 1, m, and n are each independently an integer of 1 to 6; X is a hydrogen atom, an organic group having 1 to 12 carbon atoms, a cyano group, a hydroxy group, or a halogen atom; b is an integer of 1 to 10; when b is 2 or more, a plurality of X's may be the same or different from each other; R¹ and R² are the same or different from each other, and are each a single bond or a divalent organic group.

In the formula (W-1), s is an integer of 0 to 2, preferably 0 or 1. In the formula (W-2), t is an integer of 1 to 3, preferably 1 or 2. In the formula (W-3), l, m, and n are each independently an integer of 1 to 6, and it is preferable that 1 is 2, m is 1, and n is 2.

Examples of the organic group having 1 to 12 carbon atoms as X in each of the formulas (W-1) to (W-5) include a hydrocarbon group having 1 to 12 carbon atoms and a monovalent organic group represented by —X¹—Y—X² having 1 to 12 carbon atoms, wherein X¹ is a single bond or a divalent hydrocarbon group having 1 to 11 carbon atoms, Y is —O—, —CO—, —COO—, —OCO—, —OCOO—, —NHCO—, or —CONH—, and X² is a monovalent hydrocarbon group having 1 to 12 carbon atoms.

Examples of the hydrocarbon groups having 1 to 12 carbon atoms as the aforementioned X and X² include a monovalent chain hydrocarbon group having 1 to 12 carbon atoms, a monovalent alicyclic hydrocarbon group having 3 to 12 carbon atoms, a monovalent aromatic hydrocarbon group having 6 to 12 carbon atoms, or a combination thereof.

As the chain hydrocarbon group having 1 to 12 carbon atoms above, a group corresponding to a carbon number of 1 to 12 among the monovalent chain hydrocarbon groups having 1 to 30 carbon atoms in W in formula (1) above can be suitably used.

As the alicyclic hydrocarbon groups having 3 to 12 carbon atoms above, a group corresponding to a carbon number of 3 to 12 among the monovalent alicyclic hydrocarbon groups having 3 to 20 carbon atoms in W of the formula (1) above can be suitably used.

As the monovalent aromatic hydrocarbon group having 6 to 12 carbon atoms above, a group corresponding to a carbon number of 6 to 12 among the monovalent aromatic hydrocarbon group having 6 to 20 carbon atoms in W of the formula (1) above can be suitably used.

As the divalent hydrocarbon group having 1 to 11 carbon atoms represented by X¹, groups obtained by removing one hydrogen atom from each of the groups corresponding to 1 to 11 carbon atoms among the groups recited as the hydrocarbon group having 1 to 12 carbon atoms can be suitably employed.

Examples of the halogen atom represented by X include a fluorine atom, a chlorine atom, a bromine atom, and an iodine atom, and among these, a fluorine atom and an iodine atom are preferable.

b is an integer of 1 to 3, and is preferably 1 or 2. When b is 2 or more, a plurality of X's may be the same or different from each other.

Examples of the divalent organic groups represented by R¹ and R² include a divalent organic group having 1 to 30 carbon atoms, and specifically include a divalent hydrocarbon group having 1 to 30 carbon atoms, a group in which the hydrocarbon group contains a divalent hetero atom-containing group between adjacent carbon atoms or at one terminal of the hydrocarbon group, a group obtained by replacing some or all of the hydrogen atoms of the group or the hydrocarbon group with a monovalent hetero atom-containing group.

Examples of the divalent organic group having 1 to 30 carbon atoms include groups obtained by removing one hydrogen atom from each of the monovalent chain hydrocarbon groups having 1 to 30 carbon atoms, the monovalent alicyclic hydrocarbon groups having 3 to 20 carbon atoms, and the monovalent aromatic hydrocarbon groups having 6 to 20 carbon atoms in W of the formula (1). Examples of the divalent organic group having 1 to 30 carbon atoms further include a group obtained by removing two hydrogen atoms from the aromatic heterocyclic structure in W of the formula (1) and a group obtained by removing two hydrogen atoms from the alicyclic heterocyclic structure in W of the formula (1).

As the divalent hetero atom-containing group, the divalent hetero atom-containing group in W of the formula (1) can be suitably employed.

Examples of the monovalent hetero atom-containing group include a halogen atom such as a fluorine atom, a chlorine atom, a bromine atom, or an iodine atom, a hydroxy group, a carboxy group, a cyano group, an amino group, and a sulfanyl group (—SH).

Among them, a single bond, a divalent chain hydrocarbon group, or a group containing a divalent hetero atom-containing group between adjacent carbon atoms of the divalent chain hydrocarbon group are preferable as R¹ and R², and a single bond is more preferable.

As an embodiment in which carboxy groups and hydroxy groups are bonded directly or bonded indirectly to different cyclic structures, it is preferable that a partial structure “—W(OH)ₚ(COOH)_{q}” in the formula (1) contains
one or more groups selected from the group consisting of groups represented by formulas (W-6) to (W-9) below, and
also contains one or more groups selected from the group consisting of groups represented by formulas (W-10) to (W-13) below.

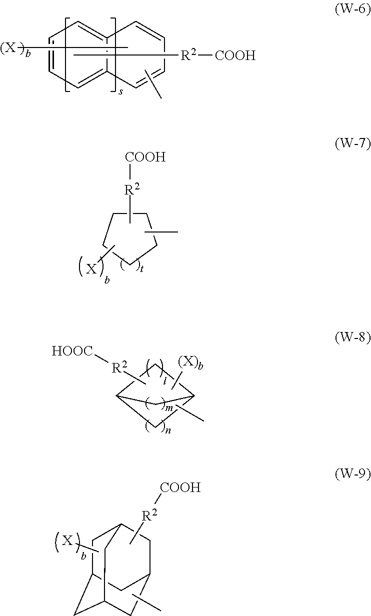

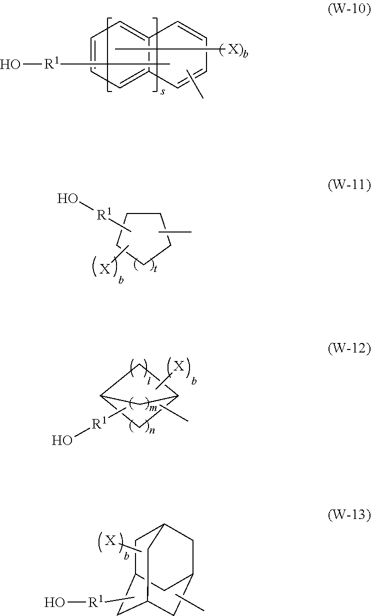

s, t, l, m, n, X, b, R¹, and R² in the formulas (W-6) to (W-13), have the same meanings as those in the formulas (W-1) to (W-5).

In addition, one or more groups selected from the group consisting of the groups represented by the formulas (W-6) to (W-9) and one or more groups selected from the group consisting of the groups represented by the formulas (W-10) to (W-13) may be bonded with a divalent organic group interposed therebetween.

Examples of such a divalent organic group include a divalent organic group represented by —X¹—Y—X¹—. X¹ and Y have the same meanings as those in the formulas (W-1) to (W-5). The two X¹'s may be the same or different. Specifically, examples of the divalent organic group include —CH₂OC(═O)— and —OC(═O)—.

The L in the formula (1) is a (r+1)-valent linking group, r is 1 to 3, and preferably is 1 or 2. As to p and q, when r is 1, both p and q are 1 to 3, and when r is 2 or 3, each of a plurality of p's and a plurality of q's is 0 to 3. It is noted that when r is 2 or 3, at least one of a plurality of p's is 1 or more and at least one of the plurality of q's is 1 or more.

Examples of wherein the (r+1)-valent linking group represented by L include a group containing one or more bonding groups selected from the group consisting of an ether bond, an amide bond, an ester bond, and an acetal bond.

The L is preferably at least one structure selected from among structures represented by formulas (L-1) to (L-5) below.

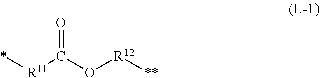

in the formula (L-1), R¹¹ is a single bond or a substituted or unsubstituted divalent hydrocarbon group having 1 to 12 carbon atoms; R¹² is a substituted or unsubstituted divalent hydrocarbon group having 1 to 12 carbon atoms; * is a bond bonding to W in the formula (1), and ** is a bond bonding to S of SO₃⁻ in the formula (1),

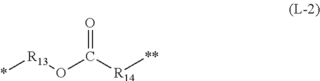

in the formula (L-2), R¹³ is a single bond or a substituted or unsubstituted divalent hydrocarbon group having 1 to 12 carbon atoms; R¹⁴ is a substituted or unsubstituted divalent hydrocarbon group having 1 to 12 carbon atoms; * is a bond bonding to W in the formula (1), and ** is a bond bonding to S of SO₃⁻ in the formula (1),

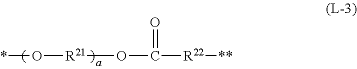

in the formula (L-3), R²¹ and R²² are the same or different from each other and are each a substituted or unsubstituted divalent hydrocarbon group having 1 to 12 carbon atoms, and a is an integer of 1 to 3; * is a bond bonding to W in the formula (1), and ** is a bond bonding to S of SO₃⁻ in the formula (1),

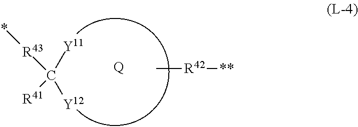

in the formula (L-4), Y¹¹ and Y¹² are each independently an oxygen atom or a sulfur atom; R⁴¹ is a hydrogen atom, a substituted or unsubstituted monovalent hydrocarbon group having 1 to 10 carbon atoms, or a monovalent organic group represented by —X¹—Y—X² and having 1 to 12 carbon atoms, wherein X¹ is a single bond or a divalent hydrocarbon group having 1 to 11 carbon atoms, Y is —O—, —CO—, —COO—, —OCO—, —OCOO—, —NHCO—, or —CONH—, and X² is a substituted or unsubstituted monovalent hydrocarbon group having 1 to 12 carbon atoms; R 42 is a single bond or a substituted or unsubstituted divalent hydrocarbon group having 1 to 10 carbon atoms; R⁴³ is a single bond or a divalent organic group; Q is a cyclic (thio) acetal structure that forms a monocyclic ring or a condensed ring together with Y¹¹, Y¹², and a carbon atom to which Y¹¹ and Y¹² are bonded; * is a bond bonding to W in the formula (1), and ** is a bond bonding to S of SO₃⁻ in the formula (1),

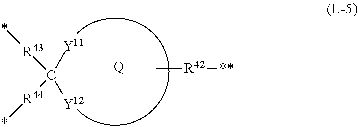

in the formula (L-5), Y¹¹, Y¹², R⁴², R⁴³, and Q each have the same meaning as in the formula (L-4); R⁴⁴ is a single bond or a divalent organic group; * is a bond bonding to W in the formula (1), and ** is a bond bonding to S of SO₃⁻ in the formula (1).

As the divalent hydrocarbon groups having 1 to 12 carbon atoms represented by R¹¹, R¹², R¹³, R¹⁴, R²¹, and R²² in the formulas (L-1) to (L-3), groups obtained by removing one hydrogen atom from each of the groups recited as the hydrocarbon group having 1 to 12 carbon atoms as the X's in the formulas (W-1) to (W-5) can be suitably employed.

As the monovalent hydrocarbon group having 1 to 10 carbon atoms represented by R⁴¹ in the formula (L-4), among the groups recited as the hydrocarbon group having 1 to 12 carbon atoms as the X's in the formulas (W-1) to (W-5), groups corresponding to 1 to 10 carbon atoms can be suitably employed.

The monovalent organic group having 1 to 12 carbon atoms represented by —X¹—Y—X² in the formula (L-4) has the same meaning as those in the formulas (W-1) to (W-5).

As the divalent hydrocarbon group having 1 to 10 carbon atoms represented by R⁴² in the formulas (L-4) and (L-5), groups obtained by removing one hydrogen atom from each of the groups corresponding to 1 to 10 carbon atoms among the groups recited as the hydrocarbon group having 1 to 12 carbon atoms as the X's in the formulas (W-1) to (W-5) can be suitably employed.

As the divalent organic groups represented by R⁴³ and R⁴⁴ in the formulas (L-4) and (L-5), the divalent organic group as R¹ in the formulas (W-1) to (W-5) can be suitably employed.

Examples of the substituent that replaces some or all of the hydrogen atoms of the hydrocarbon group include the substituents in W of the formula (1).

The L may contain a cyclic structure, and the cyclic structure of the L and the cyclic structure of W may form together a spiro ring structure. Specifically, for example, when W has a cyclohexane ring structure and L has a cyclic (thio) acetal structure represented by the formula (L-4), the spiro ring structure described below is formed. However, this is an example of forming a spiro ring structure, and an embodiment in which a spiro ring structure is formed is not limited thereto.

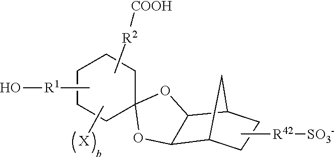

wherein, R¹, R², X, and b have the same meanings as those of the formulas (W-1) to (W-4), and R⁴² has the same meaning as that of the formula (L-4).

In order for the onium salt compound (1) to sufficiently function as a radiation-sensitive strong acid generator, a fluorine or a fluorinated hydrocarbon group is preferably bonded to a carbon atom adjacent to the sulfur atom of the sulfonate ion (SO₃⁻).

Specific examples of the anion of the onium salt compound (1) as a radiation-sensitive strong acid generator include, but are not limited to, structures of formulae (1-1-1) to (Jan. 1, 1936).

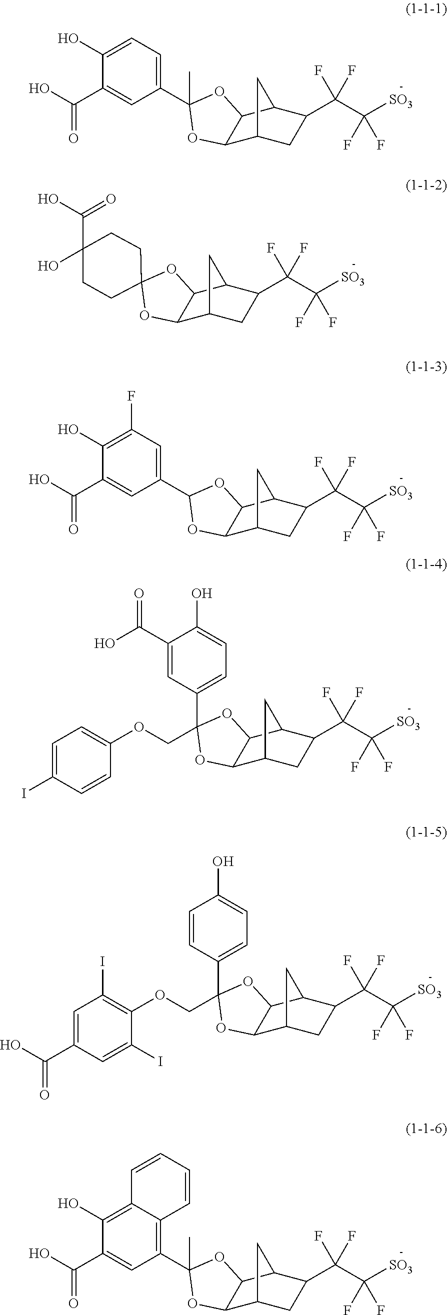

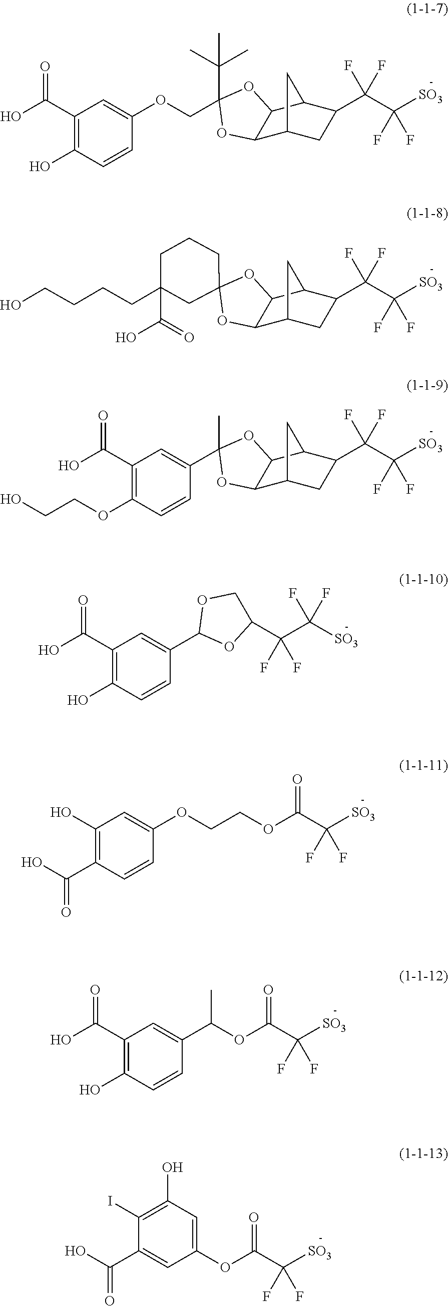

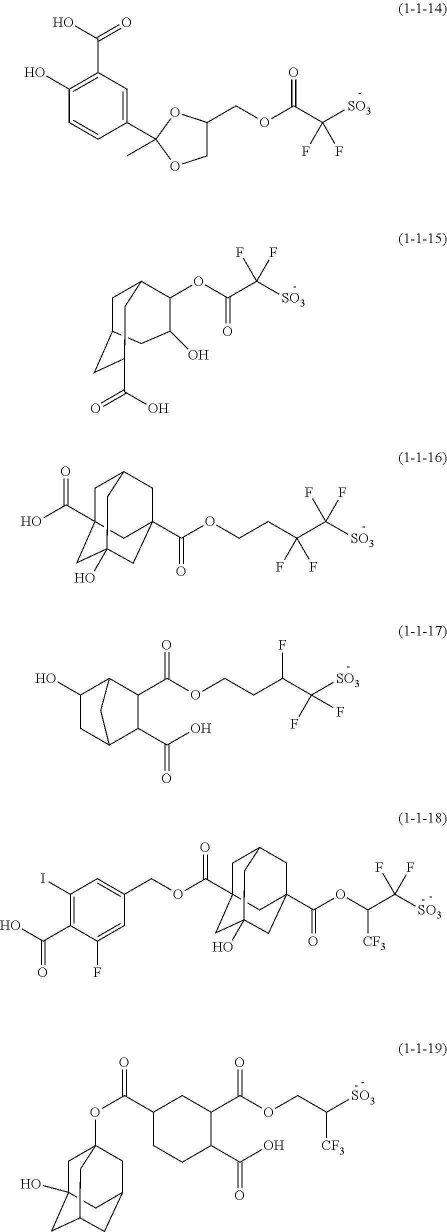

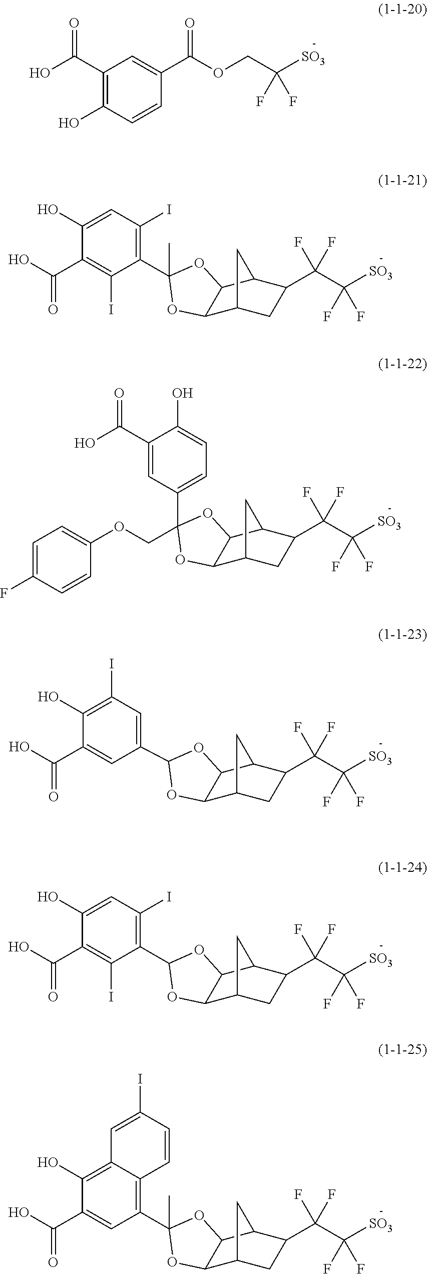

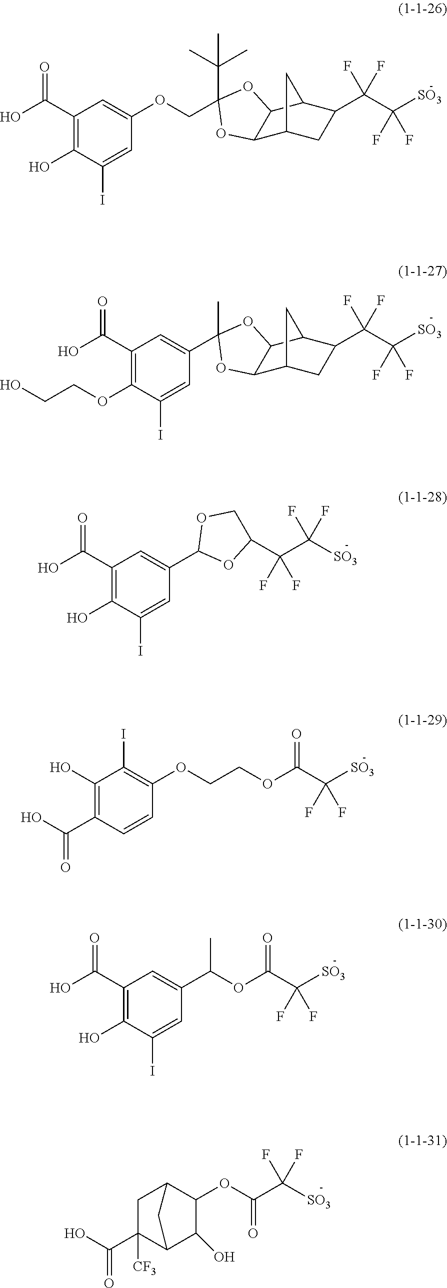

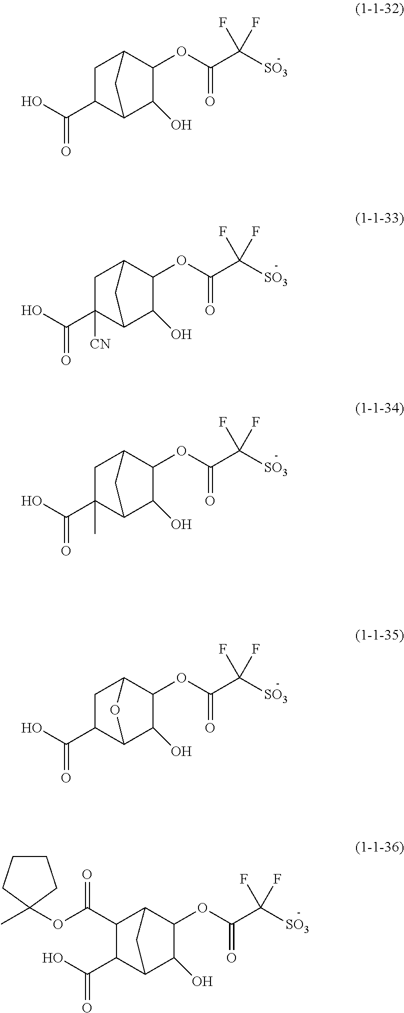

An example of the monovalent onium cation represented by M⁺ of the formula (1) is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, a iodonium cation, a phosphonium cation, a diazonium cation, and a pyridinium cation. Among 10 them, a sulfonium cation or a iodonium cation is preferred. The sulfonium cation or the iodonium cation is preferably represented by any of the formulas (X-1) to (X-6).

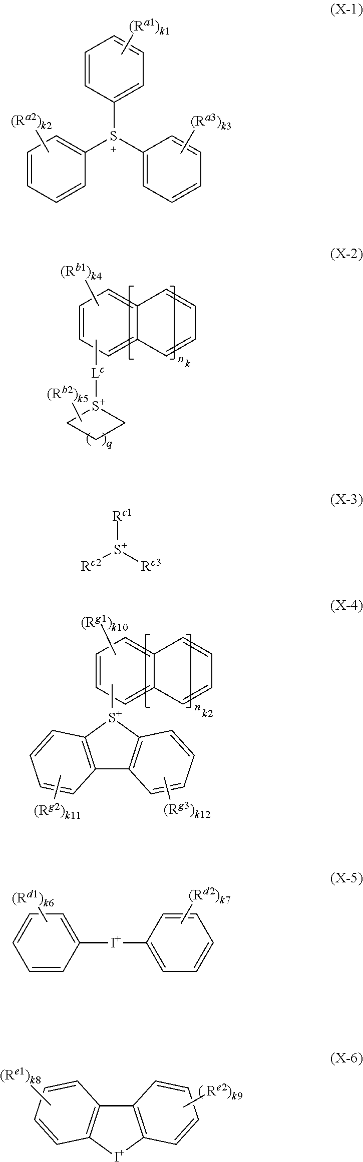

In the formula (X-1), R^{a1}, R^{a2} and R^{a3} are each independently a substituted or unsubstituted, straight or branched chain alkyl group, alkoxy group, or alkoxycarbonyloxy group having 1 to 12 carbon atoms; a substituted or unsubstituted, monocyclic or polycyclic cycloalkyl group having 3 to 12 carbon atoms; a substituted or unsubstituted aromatic hydrocarbon group having 6 to 12 carbon atoms; a hydroxy group, a halogen atom, —OSO₂—R^{P}, —SO₂-R^{Q}, —S—R^{T}, —O—, —CO— or a combination thereof; or a ring structure obtained by combining two or more of these groups. The ring structure may contain heteroatoms such as O and S between the carbon-carbon bonds forming the skeleton. R^{P}, R^{Q} and R^{T} are each independently a substituted or unsubstituted, straight or branched chain alkyl group having 1 to 12 carbon atoms; a substituted or unsubstituted alicyclic hydrocarbon group having 5 to 25 carbon atoms; or a substituted or unsubstituted aromatic hydrocarbon group having 6 to 12 carbon atoms. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the formula (X-2), R^{b1} is a substituted or unsubstituted, straight chain or branched alkyl group or alkoxy group having 1 to 20 carbon atoms; an alkoxyalkyl group; a substituted or unsubstituted acyl group having 2 to 8 carbon atoms; or a substituted or unsubstituted aromatic hydrocarbon group having 6 to 8 carbon atoms; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, straight chain or branched alkyl group having 1 to 7 carbon atoms; or a substituted or unsubstituted aromatic hydrocarbon group having 6 or 7 carbon atoms. L^{C} is a single bond or divalent linking group. k5 is an integer of 0 to 4. When there are a plurality of R^{b2}, a plurality of R^{b2} may be each identical or different. A plurality of R^{b2} may represent a ring structure obtained by combining them. q is an integer of 0 to 3. In the formula, the ring structure containing S+ may contain a heteroatom such as O or S between the carbon-carbon bonds forming the skeleton.

In the formula (X-3), R^{c1}, R^{c2} and R^{c3} are each independently a substituted or unsubstituted, straight or branched chain alkyl group having 1 to 12 carbon atoms.

In the formula (X-4), R^{g1} is a substituted or unsubstituted linear or branched alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ2 is 0 or 1. When nₖ2 is 0, k10 is an integer of 0 to 4, and when nₖ2 is 1, k10 is an integer of 0 to 7. When there are two or more R^{g1}s, the two or more R^{g1}s are the same or different from each other, and may represent a cyclic structure formed by combining them together. R^{g2} and R^{g3} are each independently a substituted or unsubstituted linear or branched alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. K11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the formula (X-5), R^{d1} and R^{d2} are each independently a substituted or unsubstituted, straight or branched chain alkyl group, alkoxy group or alkoxycarbonyl group having 1 to 12 carbon atoms; a substituted or unsubstituted aromatic hydrocarbon group having 6 to 12 carbon atoms; a halogen atom; a halogenated alkyl group having 1 to 4 carbon atoms; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the formula (X-6), R^{e1} and R^{e2} are each independently a halogen atom; a substituted or unsubstituted straight or branched chain alkyl group having 1 to 12 carbon atoms; or a substituted or unsubstituted aromatic hydrocarbon group having 6 to 12 carbon atoms. k8 and k9 are each independently an integer of 0 to 4.

Specific examples of the radiation-sensitive onium cation include, but not limited thereto, the structures represented by the formulas (1-2-1) to (Jan. 2, 1954).

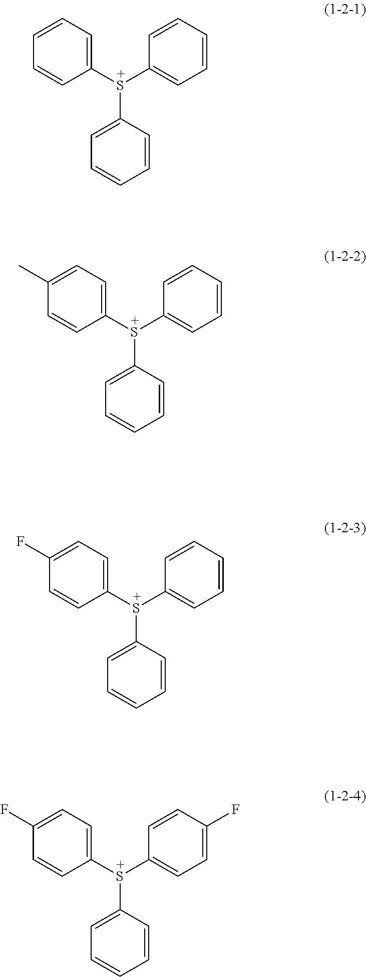

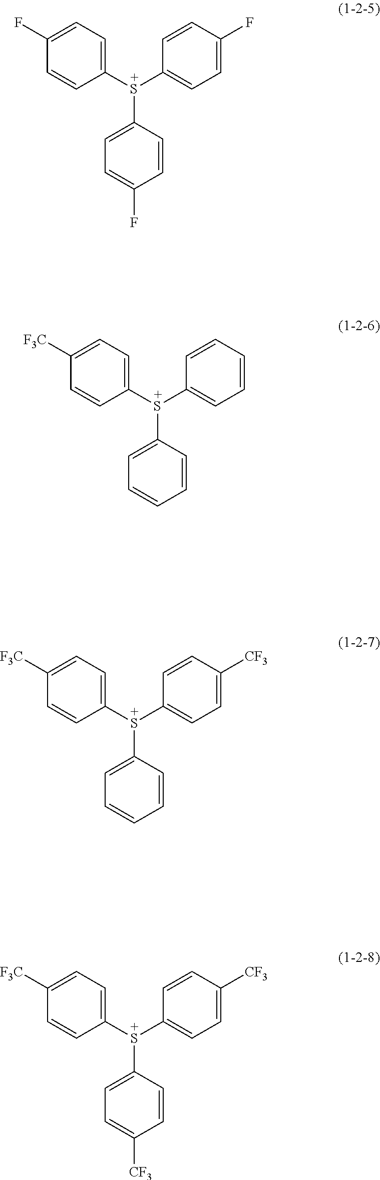

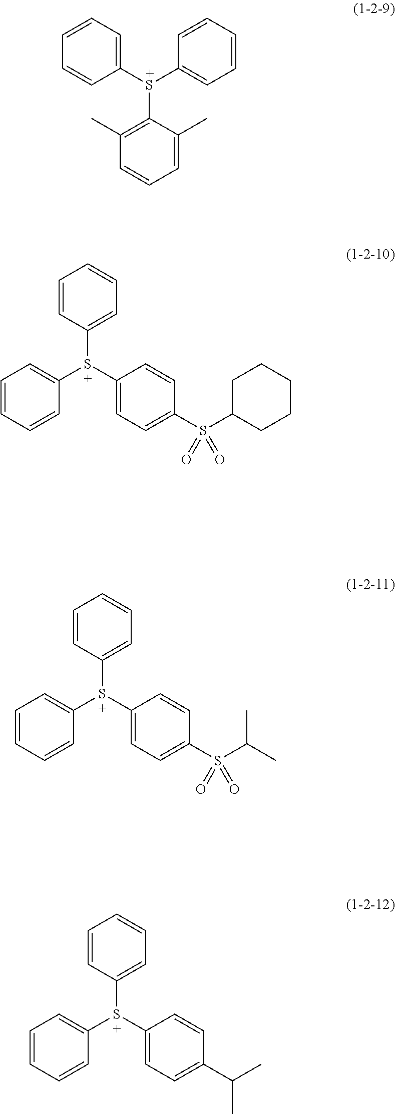

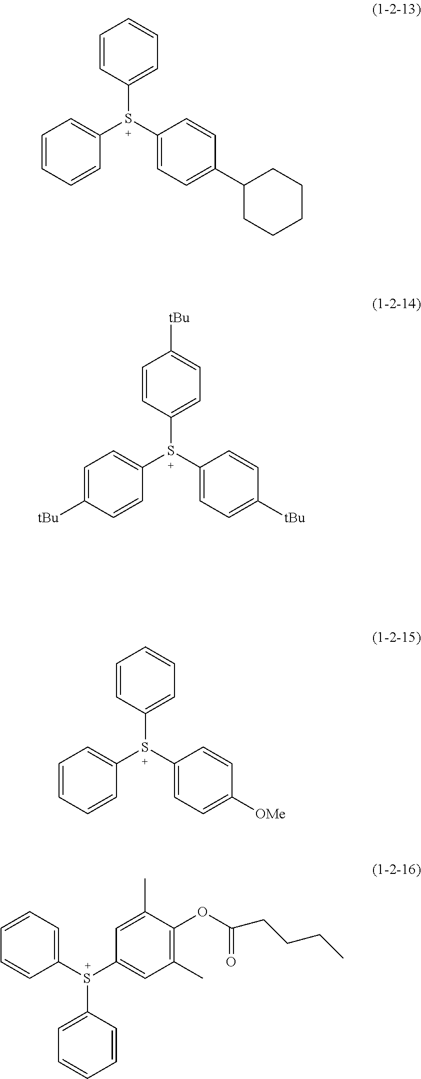

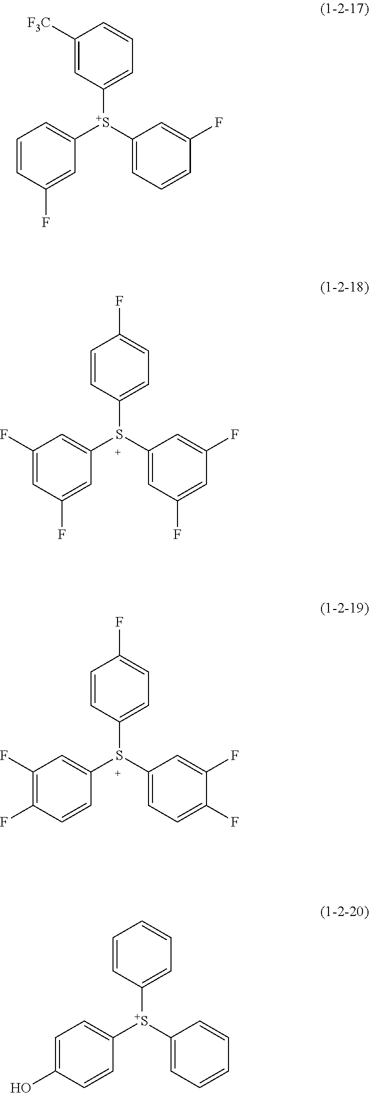

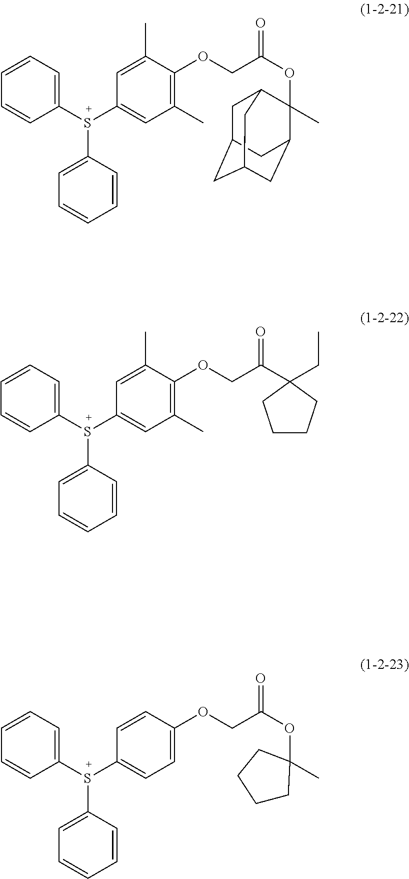

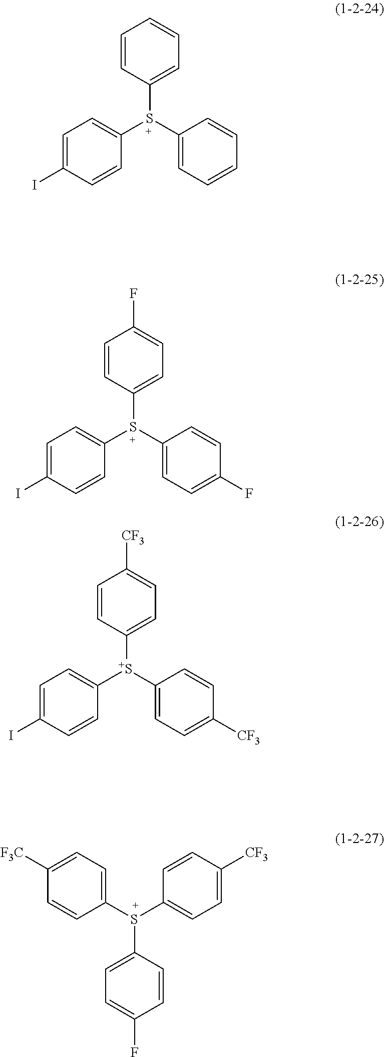

In the formula, tBu represents a t-butyl group, and Me represents a methyl group.

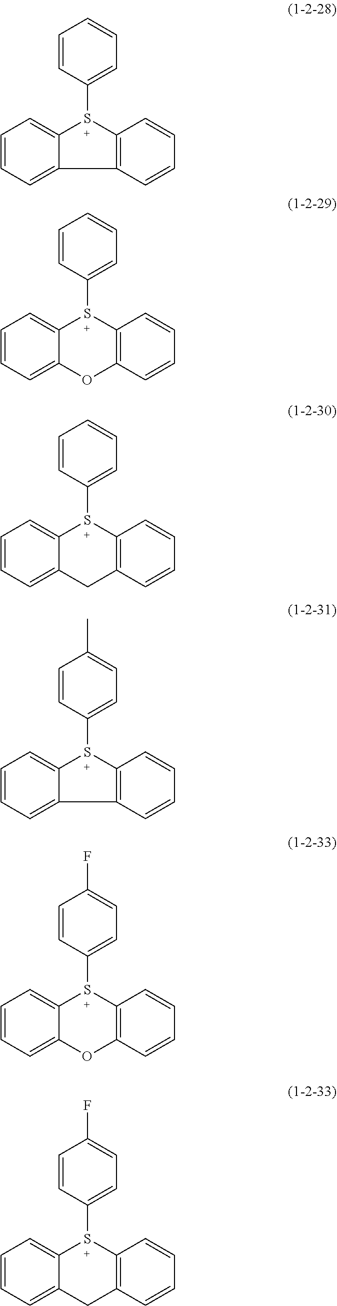

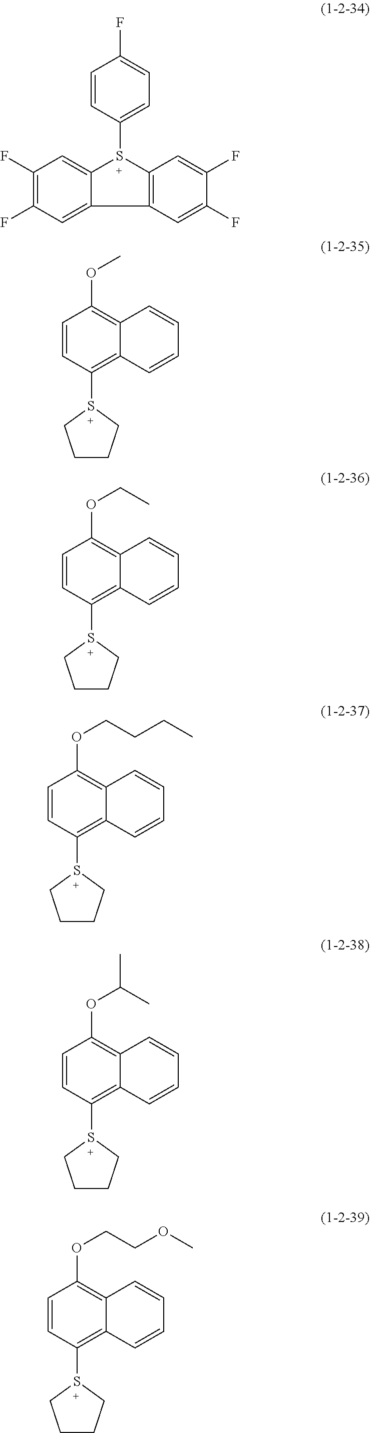

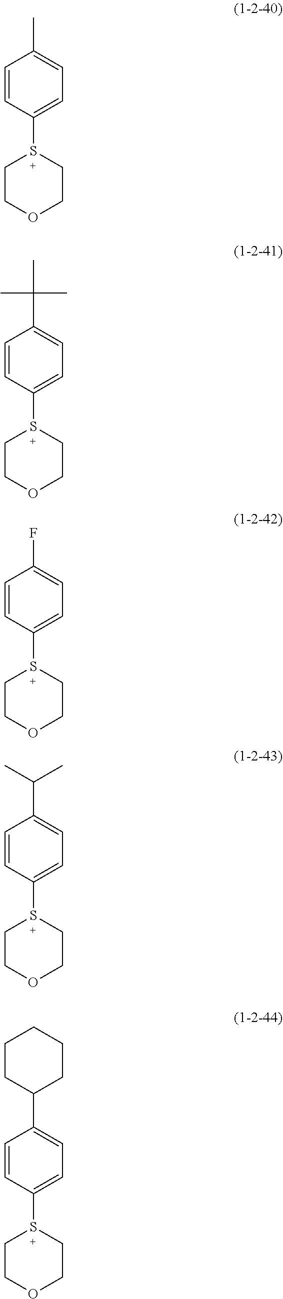

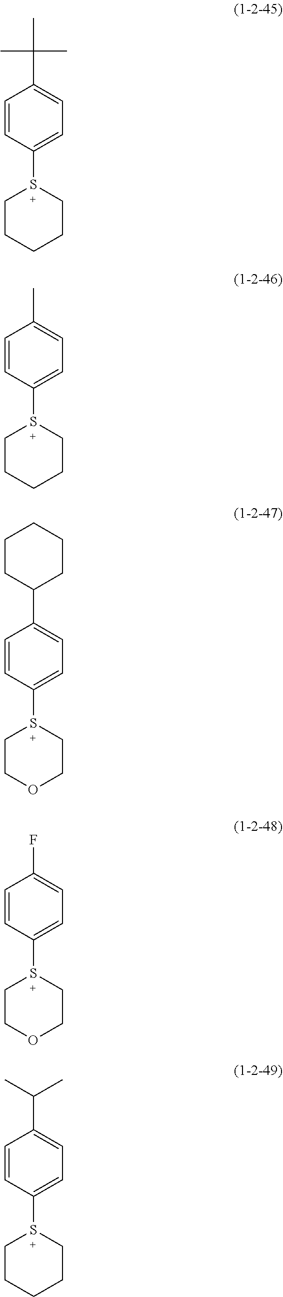

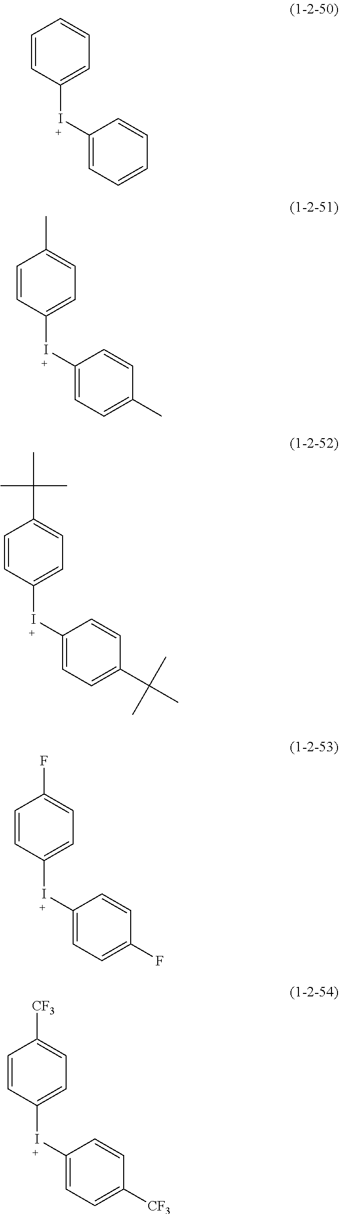

The onium salt compound (1) as a radiation-sensitive strong acid generator is obtained by appropriately combining the aforementioned anions and the aforementioned radiation-sensitive onium cations. Specific examples thereof include, but are not particularly limited to, structures represented by formulae (1-3-1) to (Jan. 3, 1936).

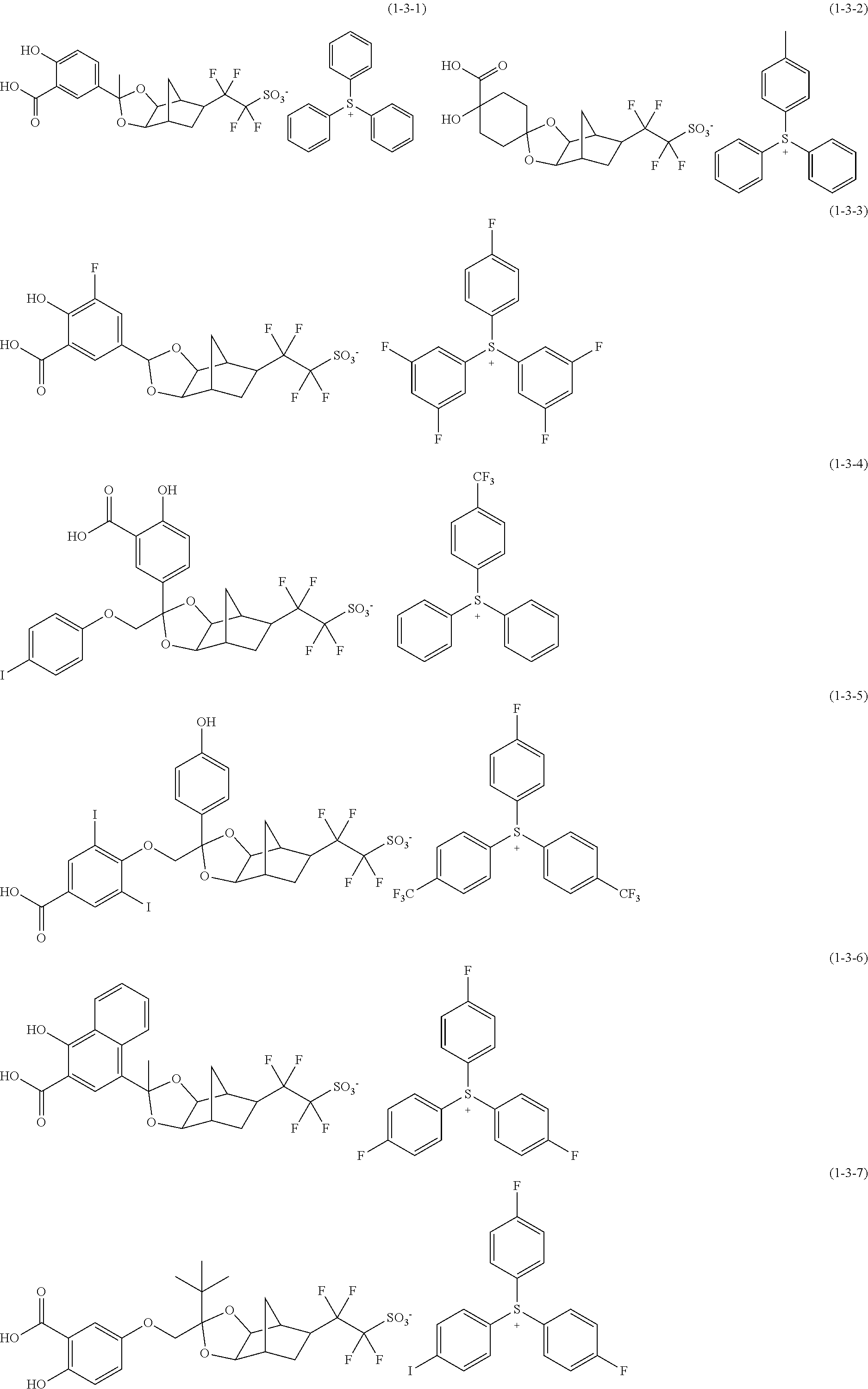

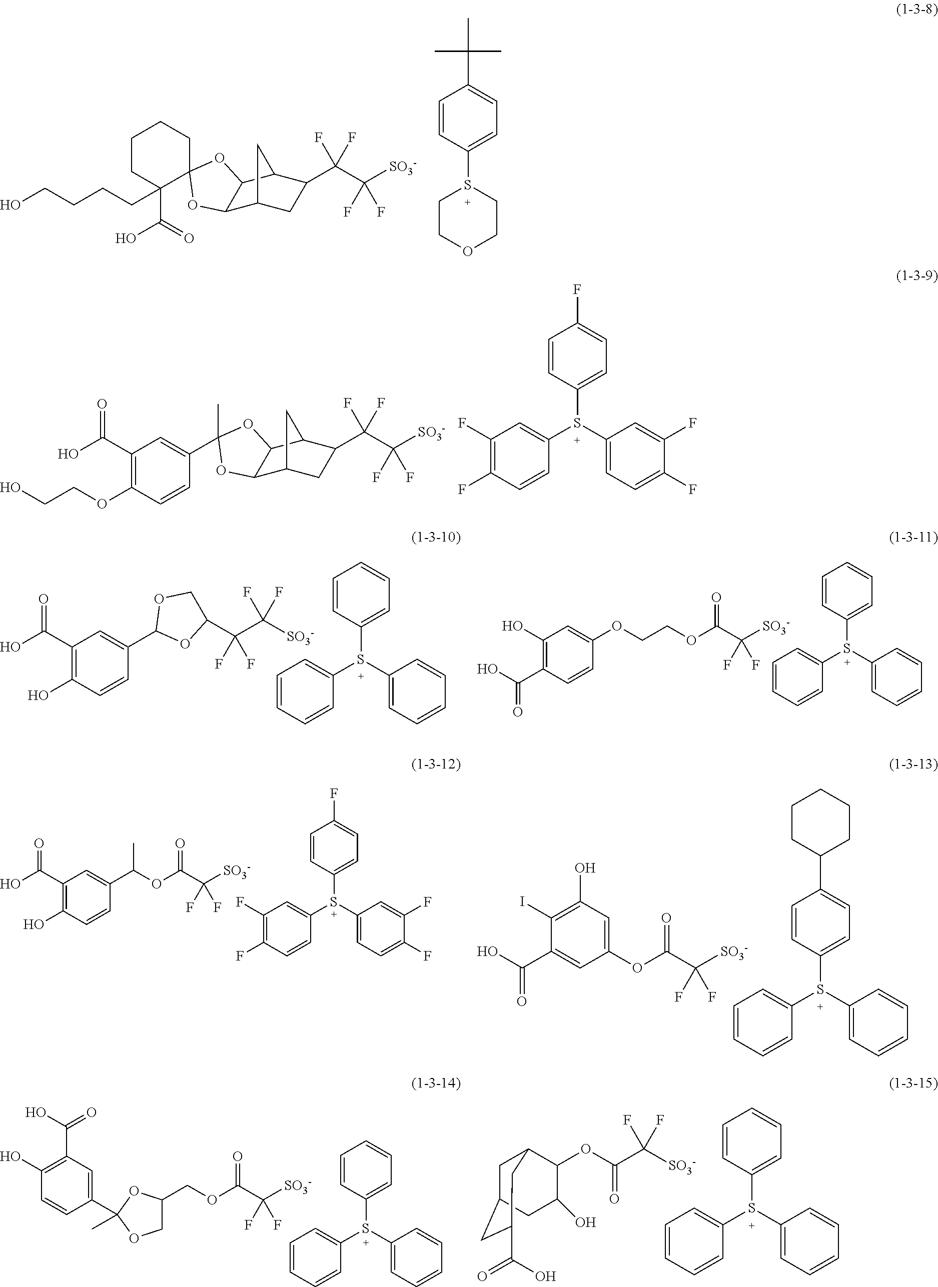

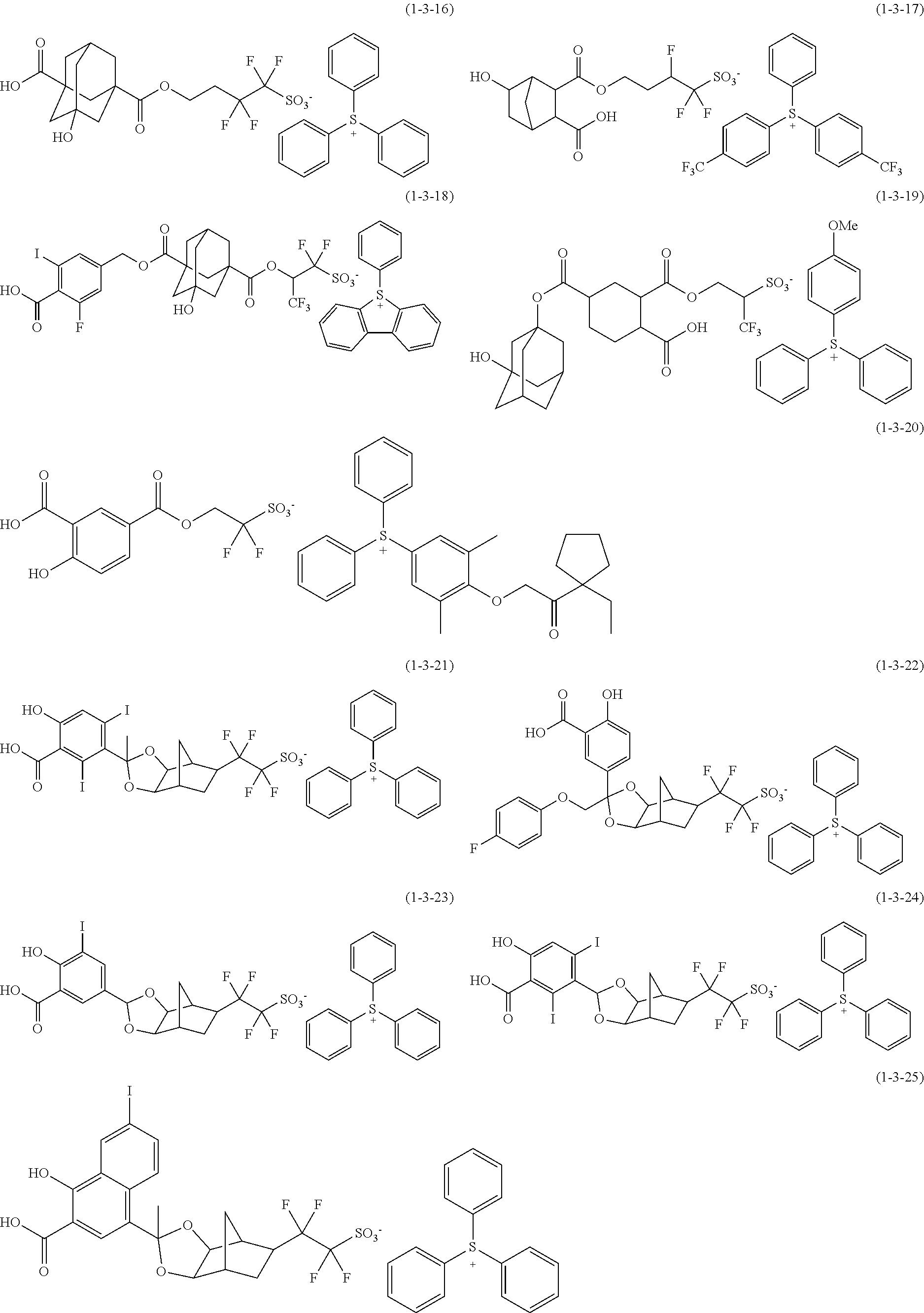

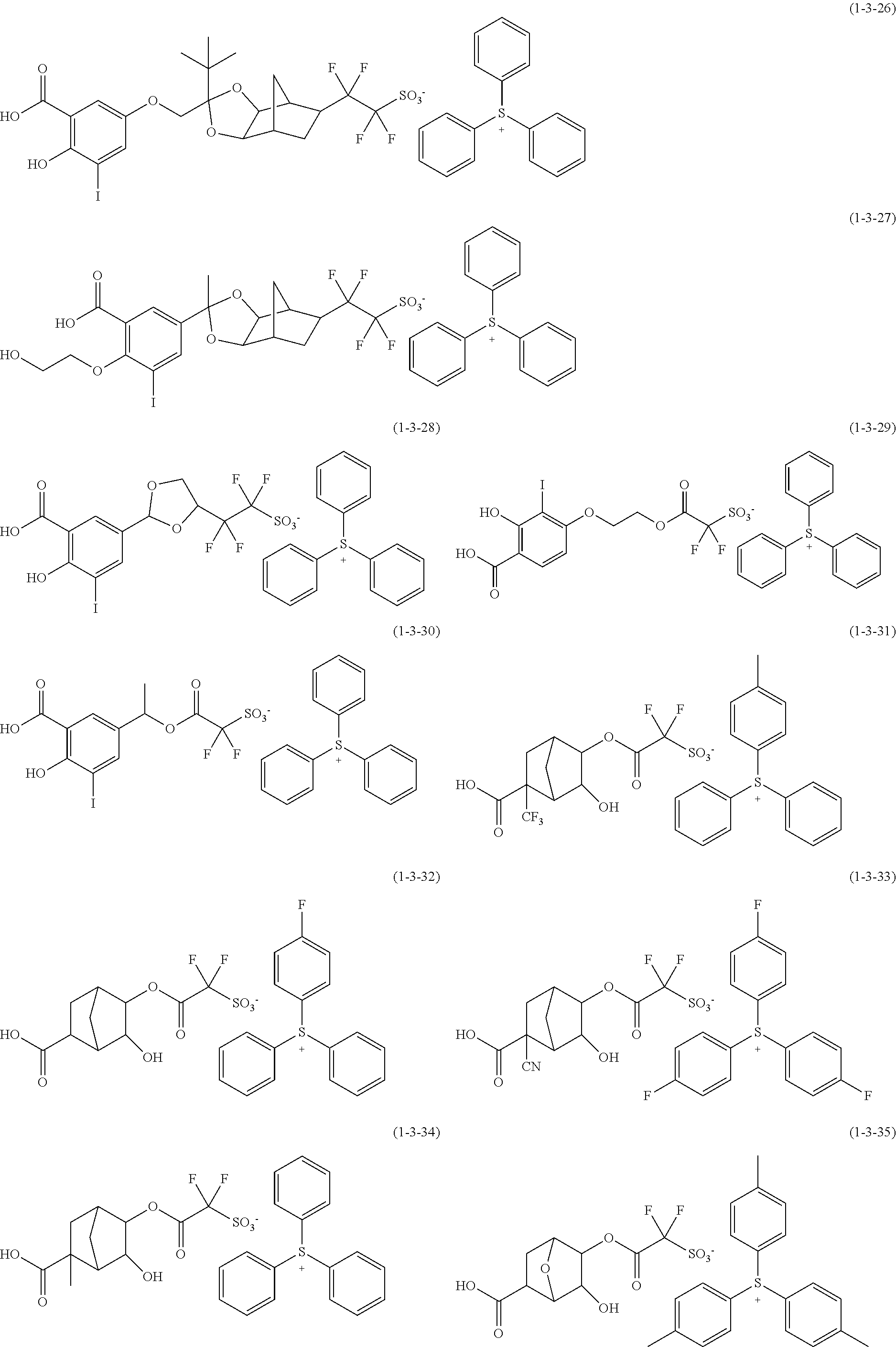

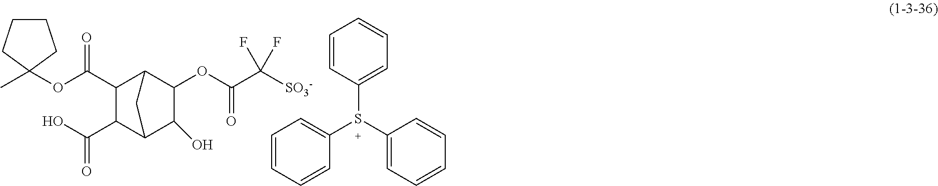

The lower limit of the content of the onium salt compound (1) as a radiation-sensitive strong acid generator (when plural kinds of onium salt compounds (1) are contained, the total content thereof) is preferably 0.1 parts by mass, more preferably 0.5 parts by mass, still more preferably 1 part by mass, and particularly preferably 3 parts by mass based on 100 parts by mass of the polymer described later. The upper limit of the content is preferably 50 parts by mass, 10 more preferably 40 parts by mass, and still more preferably 35 parts by mass. The content of the onium salt compound (1) is appropriately selected according to the type of a polymer to be used, exposure conditions, required sensitivity, and the like. This makes it possible to exhibit superior sensitivity, LWR, pattern rectangularity, development defect performance, EL, CDU, and pattern circularity when forming a resist pattern.

The onium salt compound (1) as a radiation-sensitive strong acid generator and another radiation-sensitive strong acid generator (for example, the onium salt compound (P1) described below) may be used in combination. The lower limit of the content of the onium salt compound (1) when used in combination is preferably 35% by mass, more preferably 40% by mass, and still more preferably 45% by mass based on the total mass of the radiation-sensitive strong acid generators contained in the composition. The upper limit of the content is preferably 75% by mass, more preferably 70% by mass, and still more preferably 60% by mass. This makes it possible to exhibit superior sensitivity, LWR, pattern rectangularity, EL, development defect performance, CDU, and pattern circularity when forming a resist pattern.

### (Method for Synthesizing Onium Salt Compound (1))

A representative scheme of the method for synthesizing the onium salt compound (1) is shown below.

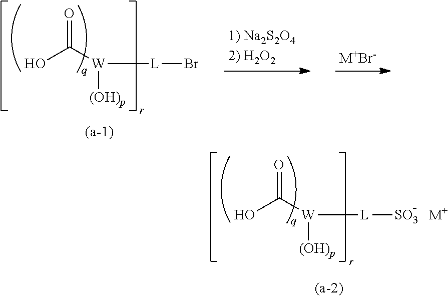

In the scheme, W, q, p, r, L, and M⁺ have the same meanings as in the formula (1).

The bromo moiety of (a-1) is converted into a sulfonate by a dithionite and an oxidizing agent, and then reacted with an onium cation halide (bromide in the scheme) corresponding to the onium cation to allow salt exchange to proceed, whereby a target onium salt compound (1) represented by formula (a-2) can be synthesized. In addition to the above, a target onium salt compound (1) can be synthesized according to the synthesis scheme described in Examples.

### (Onium Salt Compound (1) as Acid Diffusion Controlling Agent)

The onium salt compound (1) also functions as an acid diffusion controlling agent due to the structure of the onium salt compound (1). Examples of the anion of the onium salt compound (1) as an acid diffusion controlling agent include anions in which neither a fluorine atom nor a fluorinated hydrocarbon group is bonded to a carbon atom bonded to a sulfur atom of SO₃⁻ of the anion of the onium salt compound (1) as a radiation-sensitive strong acid generator.

Examples of the radiation-sensitive onium cation of the onium salt compound (1) as an acid diffusion controlling agent suitably include the same radiation-sensitive onium cations as those of the onium salt compound (1) as a radiation-sensitive strong acid generator.

The onium salt compound (1) as an acid diffusion controlling agent is obtained by appropriately combining the aforementioned anions and the aforementioned radiation-sensitive onium cations. Specific examples thereof include, but are not particularly limited to, structures represented by formulae (1-4-1) to (Jan. 4, 1934).

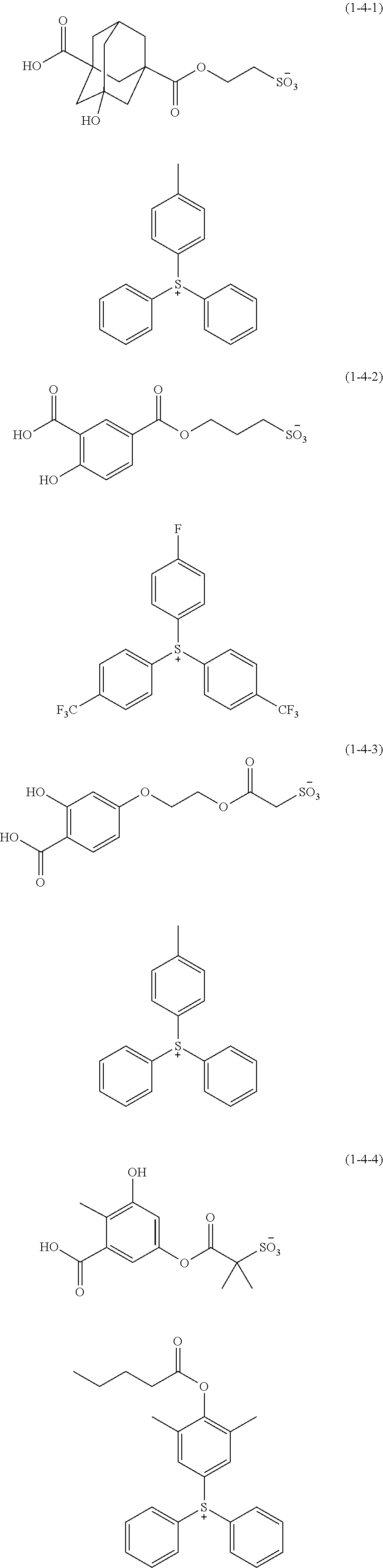

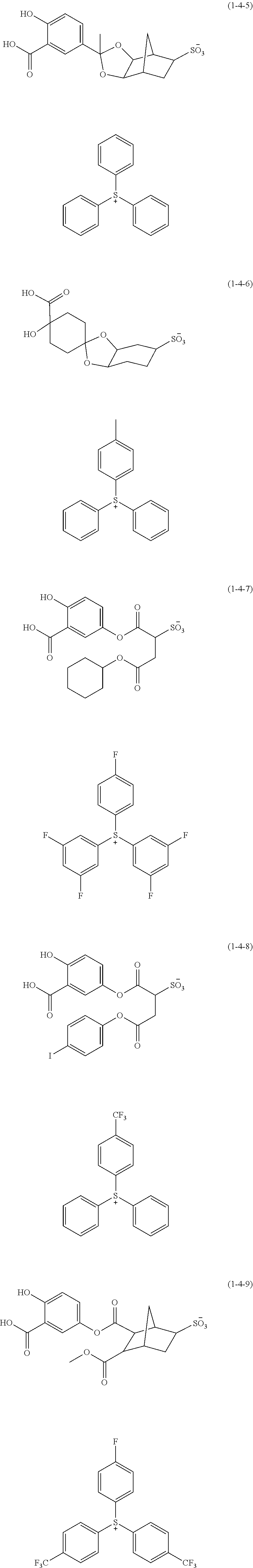

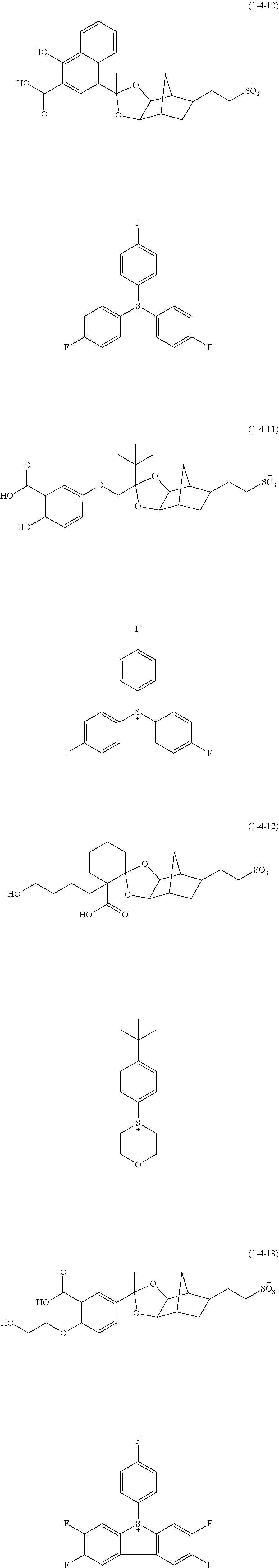

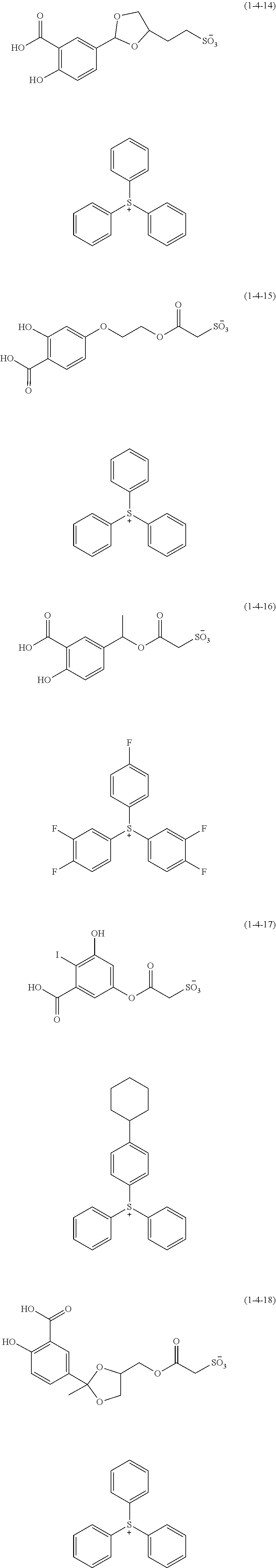

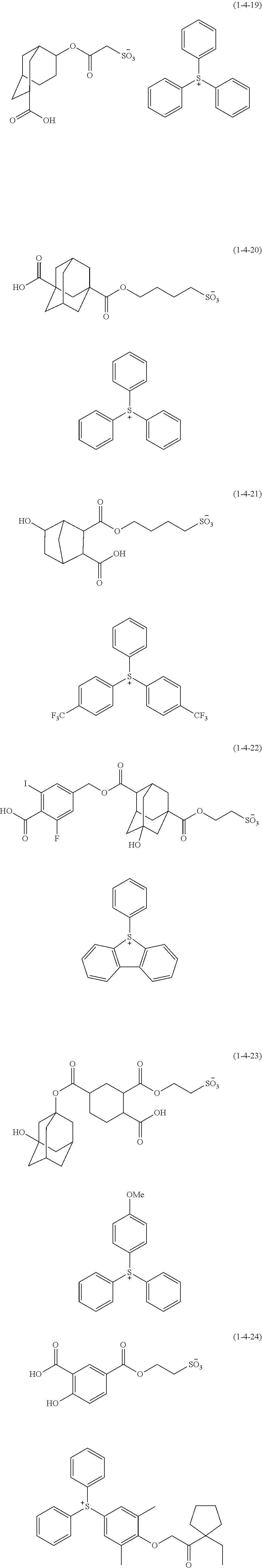

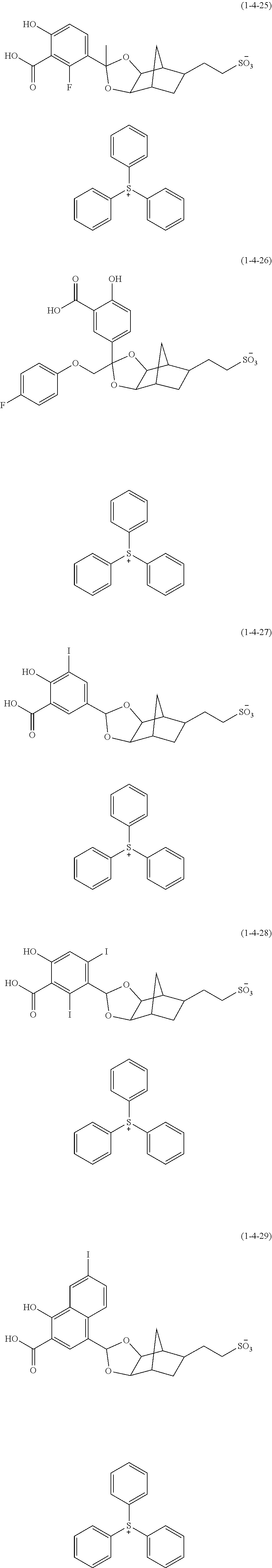

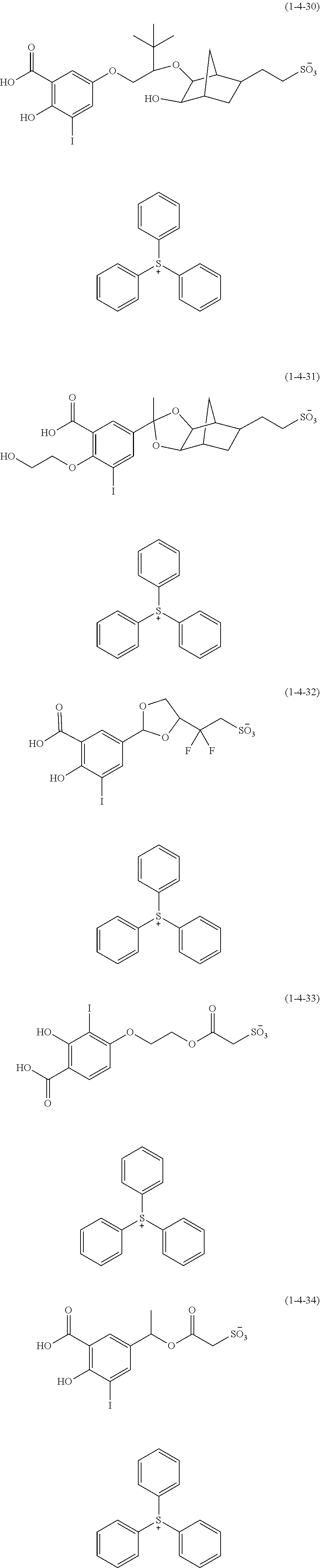

The lower limit of the content of the onium salt compound (1) as an acid diffusion controlling agent (when plural kinds of onium salt compounds (1) are contained, the total content thereof) is preferably 0.1 parts by mass, more preferably 0.5 parts by mass, still more preferably 1 part by mass, and particularly preferably 3 parts by mass based on 100 parts by mass of the polymer described later. The upper limit of the content is preferably 40 parts by mass, more preferably 30 parts by mass, still more preferably 20 parts by mass, and particularly preferably 10 parts by mass. The content of the onium salt compound (1) is appropriately selected according to the type of a polymer to be used, exposure conditions, required sensitivity, and the like. This makes it possible to exhibit superior sensitivity, LWR, pattern rectangularity, development defect performance, EL, CDU, and pattern circularity when forming a resist pattern.

The lower limit of the content of the onium salt compound (1) is preferably 0.1 parts by mass, more preferably 0.5 parts by mass, still more preferably 1 parts by mass, and particularly preferably 3 parts by mass, regardless of whether the onium salt compound (1) functions as the radiation-sensitive strong acid generator or the acid diffusion controlling agent. The upper limit of the content is preferably 40 parts by mass, more preferably 30 parts by mass, and still more preferably 20 parts by mass. This makes it possible to exhibit superior sensitivity, LWR, pattern rectangularity, development defect performance, EL, CDU, and pattern circularity when forming a resist pattern.

(Radiation-Sensitive Strong Acid Generators Other than Onium Salt Compound (1))

The radiation-sensitive composition may include a radiation-sensitive strong acid generator other than the onium salt compound (1) as a radiation-sensitive strong acid generator.

Examples of the radiation-sensitive strong acid generator include an onium salt compound (P1) represented by the following formula (P1), provided that those corresponding to the onium salt compound (1) are excluded.

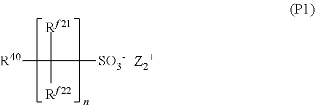

wherein,
R⁴⁰ is a monovalent organic group having 3 to 40 carbon atoms containing a cyclic structure,
R^{f21} and R^{f22} each independently represent a fluorine atom or a monovalent fluorinated hydrocarbon group, when there are a plurality of R^{f21}s and R^{f22}s, the plurality of R^{f21}s and R^{f22}s are the same or different from each other;
n is an integer of 1 to 4; and
Z₂⁺ represents a monovalent radiation-sensitive onium cation.

As the monovalent organic group having 3 to 40 carbon atoms and containing a cyclic structure that is represented by R⁴⁰, monovalent organic groups among organic groups having 3 to 40 carbon atoms and having at least one cyclic structure that are represented by W in the formula (1) can be suitably employed.

Examples of the monovalent fluorinated hydrocarbon groups represented by R^{f21} and R^{f22} include a monovalent fluorinated chain hydrocarbon group having 1 to 20 carbon atoms and a monovalent fluorinated alicyclic hydrocarbon group having 3 to 20 carbon atoms.

Examples of the monovalent fluorinated chain hydrocarbon group having 1 to 20 carbon atoms include:
fluorinated alkyl groups such as a trifluoromethyl group, a 2,2,2-trifluoroethyl group, a pentafluoroethyl group, a 2,2,3,3,3-pentafluoropropyl group, a 1,1,1,3,3,3-hexafluoropropyl group, a heptafluoro-n-propyl group, a heptafluoro-i-propyl group, a nonafluoro-n-butyl group, a nonafluoro-i-butyl group, a nonafluoro-t-butyl group, a 2,2,3,3,4,4,5,5-octafluoro-n-pentyl group, a tridecafluoro-n-hexyl group, and a 5,5,5-trifluoro-1,1-diethylpentyl group;
fluorinated alkenyl groups such as a trifluoroethenyl group and a pentafluoropropenyl group; and
fluorinated alkynyl groups such as a fluoroethynyl group and a trifluoropropynyl group.

Examples of the monovalent fluorinated alicyclic hydrocarbon group having 3 to 20 carbon atoms include:
fluorinated cycloalkyl groups such as a fluorocyclopentyl group, a difluorocyclopentyl group, a nonafluorocyclopentyl group, a fluorocyclohexyl group, a difluorocyclohexyl group, an undecafluorocyclohexylmethyl group, a fluoronorbornyl group, a fluoroadamantyl group, a fluorobornyl group, a fluoroisobornyl group, and a fluorotricyclodecyl group; and
fluorinated cycloalkenyl groups such as a fluorocyclopentenyl group and a nonafluorocyclohexenyl group.

As the fluorinated hydrocarbon group, a monovalent fluorinated chain hydrocarbon group having 1 to 8 carbon atoms is preferable, and a monovalent fluorinated linear hydrocarbon group having 1 to 5 carbon atoms is more preferable.

Specific examples of the anion of the onium salt compound (P1) include, but are not limited to, structures represented by formulas (2-1-1) to (Feb. 1, 1932).

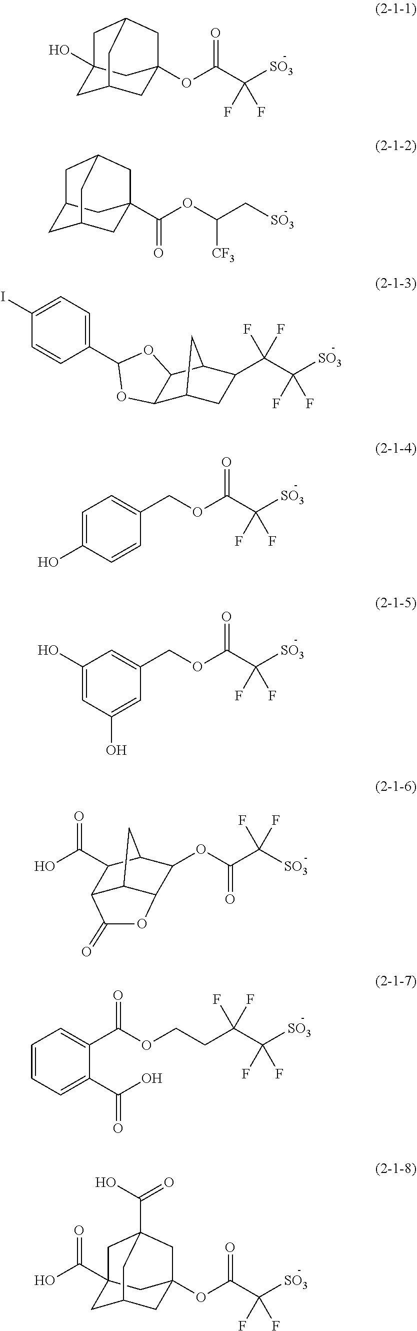

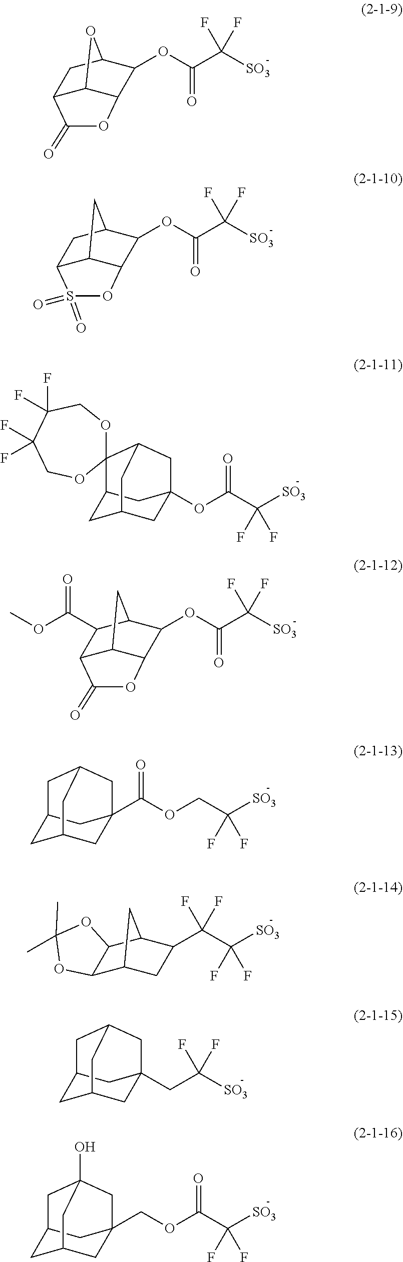

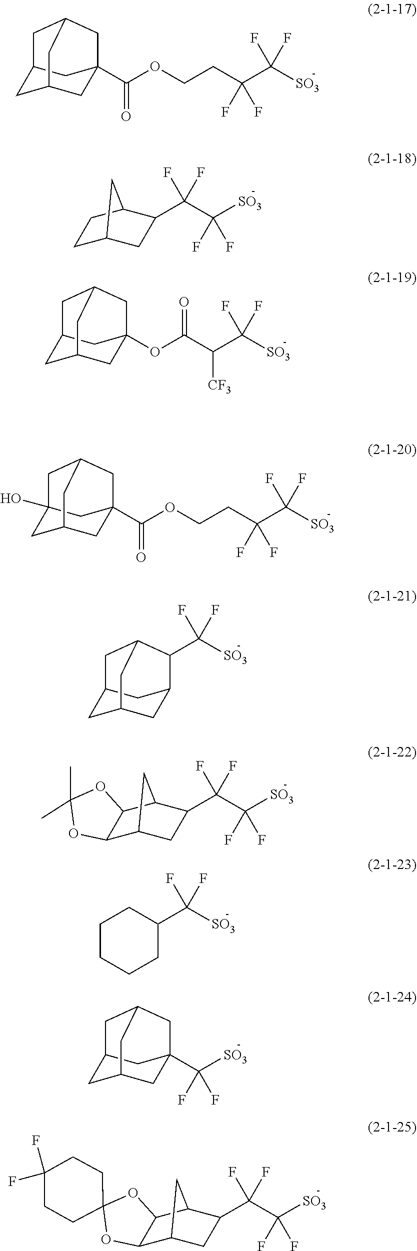

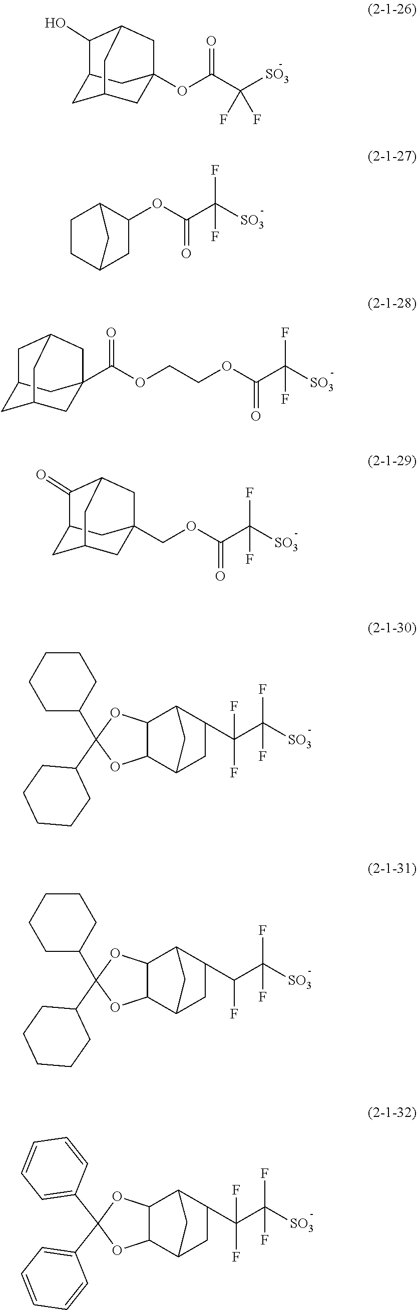

Specific examples of the radiation-sensitive onium cation of the onium salt compound (P1) are not limited, but the structures exemplified as the specific examples of the radiation-sensitive onium cation of the formula (1) can be suitably employed.

Examples of the onium salt compound (P1) include structures obtained by arbitrarily combining the anions and the radiation-sensitive onium cations. Specific examples of the onium salt compound (P1) include, but not limited thereto, onium salt compounds represented by formulas (2-1) to (2-32) below.

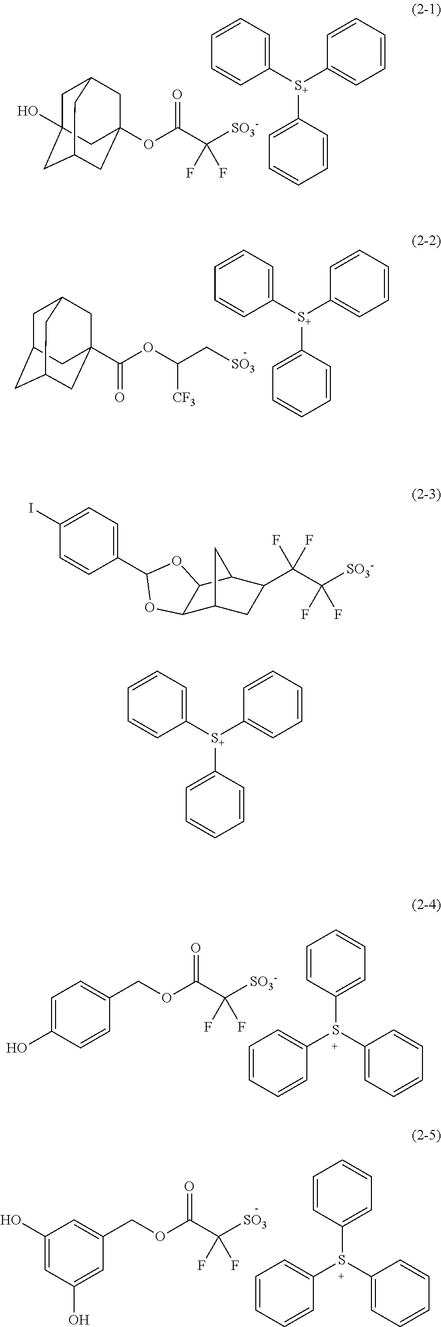

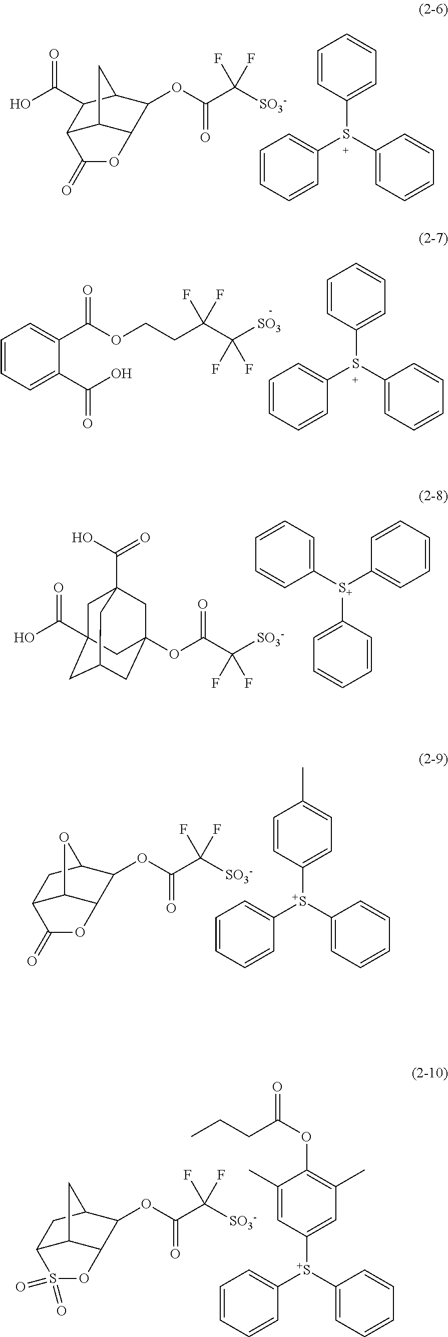

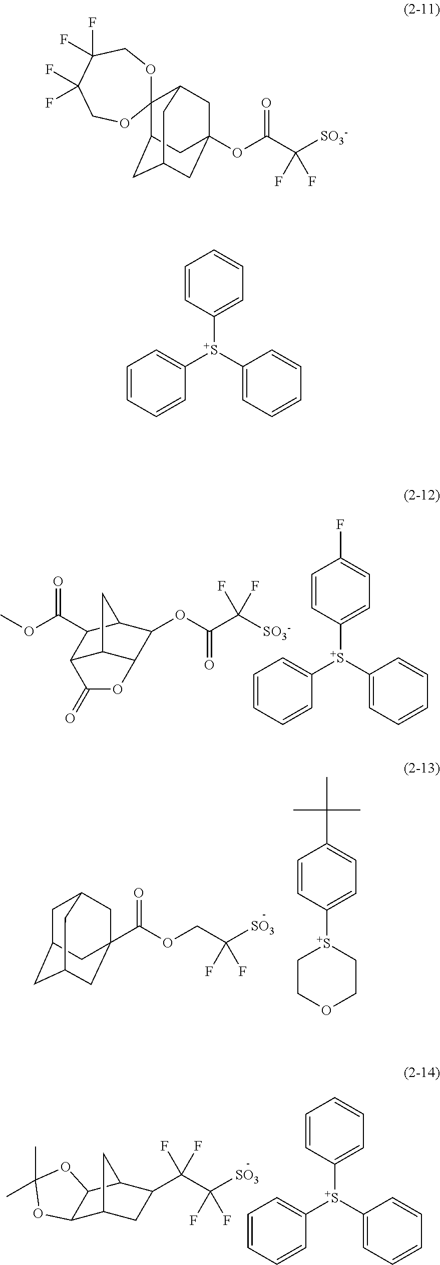

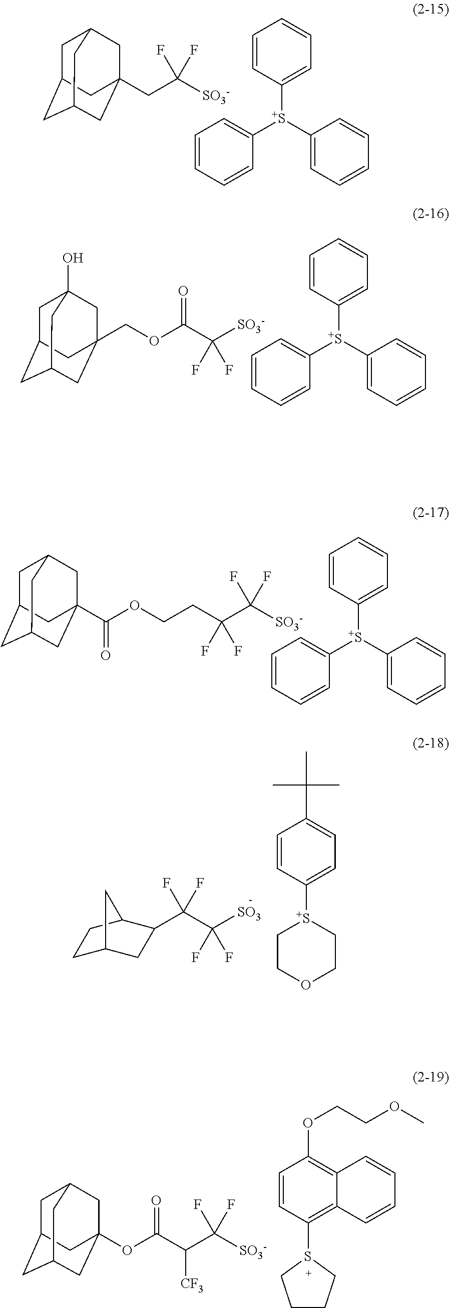

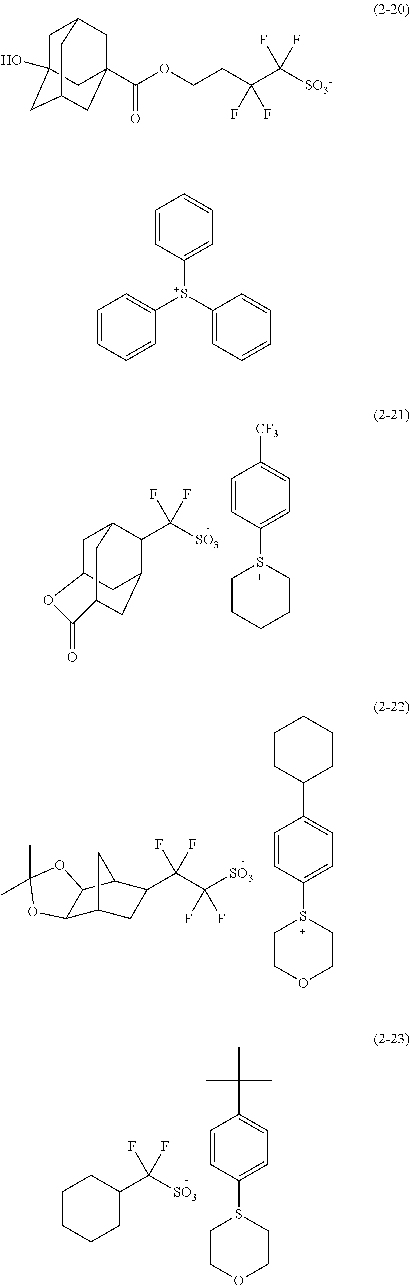

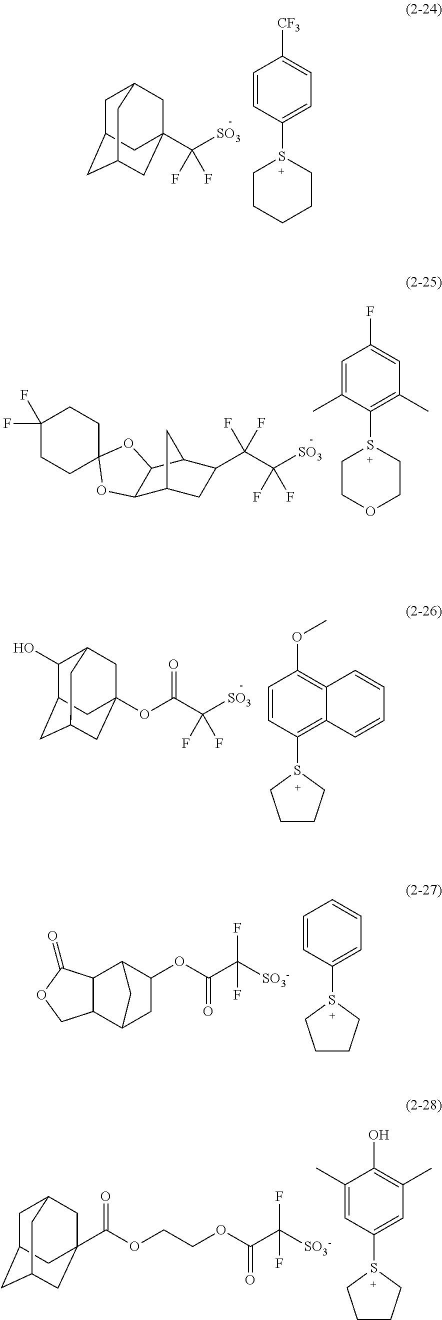

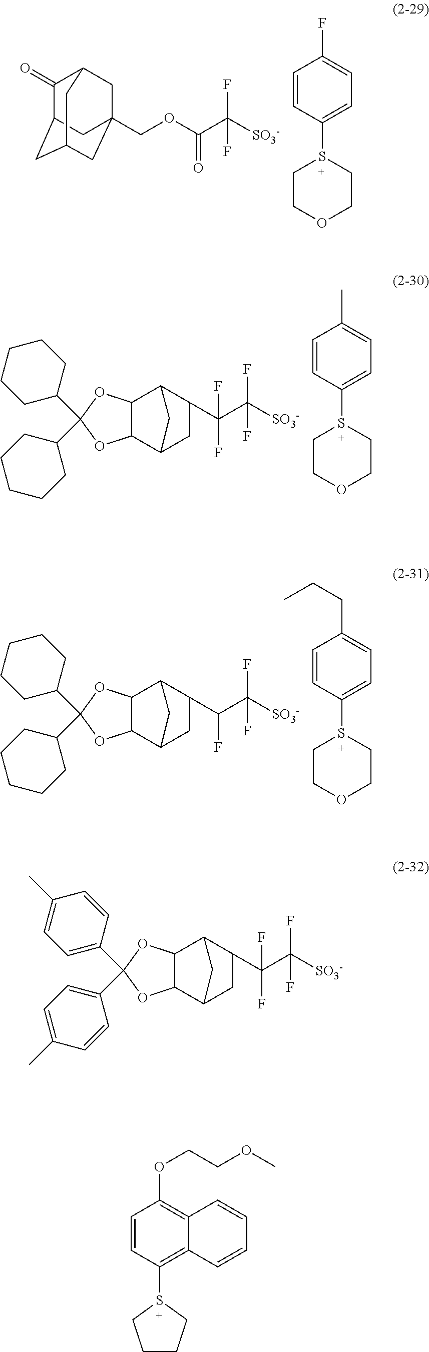

The lower limit of the content of the onium salt compound (P1) (in the case of containing a plurality of onium salt compounds (P1), the total content thereof) is preferably 0 parts by mass, more preferably 0.1 parts by mass, still more preferably 0.5 part by mass, and particularly preferably 3 parts by mass based on 100 parts by mass of a polymer to be described later. The upper limit of the content is preferably 50 parts by mass, more preferably 40 parts by mass, still more preferably 30 parts by mass, and particularly preferably 25 parts by mass. The content of the onium salt compound (P1) is appropriately selected according to the type of the polymer to be used, exposure conditions, required sensitivity, and the like.

### (Polymer)

The polymer is an aggregate of polymers having a structural unit (hereinafter, also referred to as “structural unit (I)”) containing an acid-dissociable group (hereinafter, this aggregate is also referred to as “base polymer”). The “acid-dissociable group” refers to a group that substitutes for a hydrogen atom of a carboxy group, a phenolic hydroxyl group, an alcoholic hydroxyl group, a sulfo group, or the like, and is dissociated by the action of an acid. The radiation-sensitive composition is excellent in pattern-forming performance because the polymer has the structural unit (I).

In addition to the structural unit (I), the base polymer preferably has a structural unit (II) containing at least one selected from the group consisting of a lactone structure, a cyclic carbonate structure, and a sultone structure described later, and may have another structural unit other than the structural units (I) and (II). Each of the structural units will be described below.

### [Structural Unit (I)]

The structural unit (I) contains an acid-dissociable group. The structural unit (I) is not particularly limited as long as it contains an acid-dissociable group. Examples of such a structural unit (I) include a structural unit having a tertiary alkyl ester moiety, a structural unit having a structure obtained by substituting the hydrogen atom of a phenolic hydroxyl group with a tertiary alkyl group, and a structural unit having an acetal bond. From the viewpoint of improving the pattern-forming performance of the radiation-sensitive composition, a structural unit represented by the formula (2) (hereinafter also referred to as a “structural unit (I-1)”) is preferred.

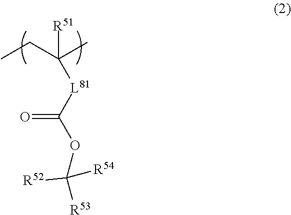

In the formula (2), R⁵¹ is a hydrogen atom, a fluorine atom, a methyl group, or a trifluoromethyl group, R⁵² is a substituted or unsubstituted monovalent hydrocarbon group having 1 to 20 carbon atoms, R⁵³ and R⁵⁴ are each independently a monovalent chain hydrocarbon group having 1 to 10 carbon atoms or a monovalent alicyclic hydrocarbon group having 3 to 20 carbon atoms or R⁵³ and R⁵⁴ taken together represent a divalent alicyclic group having 3 to 20 carbon atoms together with the carbon atom to which R⁵³ and R⁵⁴ are bonded. L⁸¹ is a single bond or a divalent organic group.

From the viewpoint of copolymerizability of a monomer that will give the structural unit (I-1), R⁵¹ is preferably a hydrogen atom or a methyl group, more preferably a methyl group.

Examples of the monovalent hydrocarbon group having 1 to 20 carbon atoms represented by R⁵² include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

As the chain hydrocarbon group having 1 to 10 carbon atoms represented by R⁵² to R⁵⁴, a group corresponding to a carbon number of 1 to 10 among the monovalent chain hydrocarbon groups having 1 to 30 carbon atoms in W in formula (1) above can be suitably used.

As the alicyclic hydrocarbon groups having 3 to 20 carbon atoms represented by R⁵² to R⁵⁴, the monovalent alicyclic hydrocarbon groups having 3 to 20 carbon atoms in W of the formula (1) can be suitably employed.

As the monovalent aromatic hydrocarbon group having 6 to 20 carbon atoms represented by R⁵², the monovalent aromatic hydrocarbon groups having 6 to 20 carbon atoms in W in the formula (1) can be suitably employed.

R⁵² is preferably a linear or branched saturated hydrocarbon group having 1 to 10 carbon atoms or an alicyclic hydrocarbon group having 3 to 20 carbon atoms.

The divalent alicyclic group having 3 to 20 carbon atoms formed by the chain hydrocarbon group or the alicyclic hydrocarbon group represented by R⁵³ and R⁵⁴ taken together with the carbon atom to which R⁵³ and R⁵⁴ are bonded is not particularly limited as long as it is a group obtained by removing two hydrogen atoms from the same carbon atom constituting a carbon ring of a monocyclic or polycyclic alicyclic hydrocarbon having the above-described carbon number. The divalent alicyclic group having 3 to 20 carbon atoms may either be a monocyclic hydrocarbon group or a polycyclic hydrocarbon group. The polycyclic hydrocarbon group may either be a bridged alicyclic hydrocarbon group or a condensed alicyclic hydrocarbon group and may either be a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between two adjacent carbon atoms).

When the monocyclic alicyclic hydrocarbon group is a saturated hydrocarbon group, preferred examples thereof include a cyclopentanediyl group, a cyclohexanediyl group, a cycloheptanediyl group, and a cyclooctanediyl group. When the monocyclic alicyclic hydrocarbon group is an unsaturated hydrocarbon group, preferred examples thereof include a cyclopentenediyl group, a cyclohexenediyl group, a cycloheptenediyl group, a cyclooctenediyl group, and a cyclodecenediyl group. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group).

Among them, R⁵² is preferably an alkyl group having 1 to 4 carbon atoms, and the alicyclic structure formed by R⁵³ and R⁵⁴ combined together and a carbon atom to which they are bonded is preferably a polycyclic or monocyclic cycloalkane structure.

Examples of the substituent that replaces some or all of the hydrogen atoms of the hydrocarbon group include the substituents in W of the formula (1).

As the divalent organic groups represented by L⁸¹, the divalent organic group as R¹ in the formulas (W-1) to (W-5) can be suitably employed.

Examples of the structural unit (1-1) include structural units represented by the formulas (3-1) to (3-9) (hereinafter also referred to as “structural units (1-1-1) to (I-1-9)”).

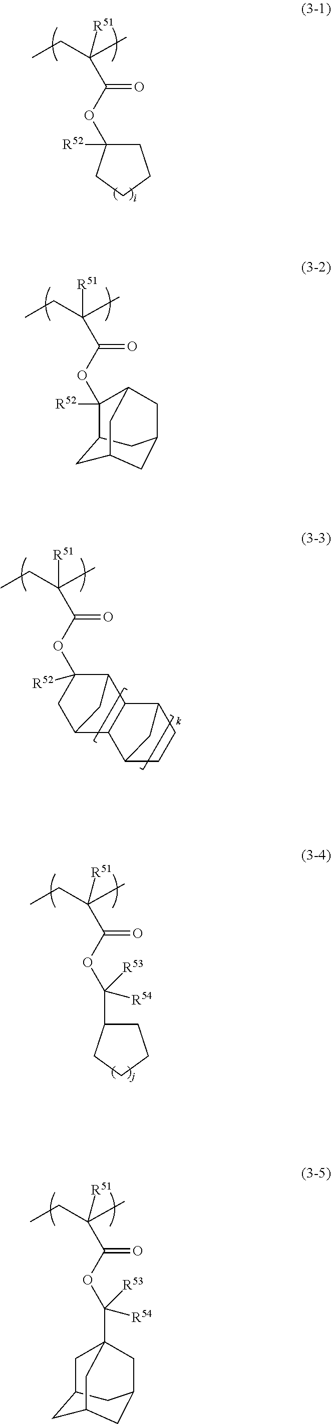

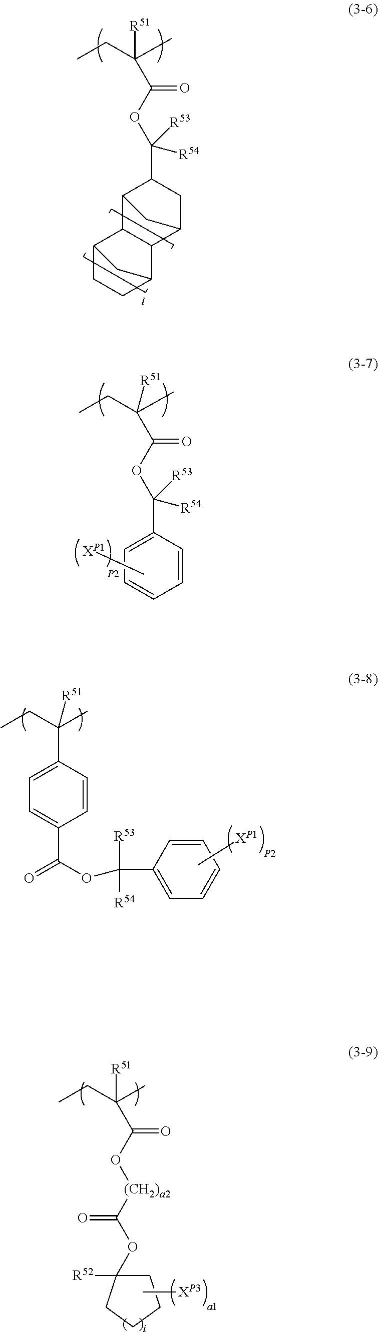

In the formulas (3-1) to (3-9), R⁵¹ to R⁵⁴ have the same meaning as in the formula (2), i and j are each independently an integer of 1 to 4, and k and l are each 0 or 1.

In the formulas (3-1) to (3-9), i and j are preferably 1, and R⁵² is preferably a methyl group, an ethyl group, an isopropyl group or a cyclopentyl group. R⁵³ and R⁵⁴ are each preferably a methyl group, or an ethyl group.

In the formulas (3-1) to (3-9), X^{P1} is a halogen atom such as a fluorine atom, a chlorine atom, a bromine atom, or an iodine atom. P2 is an integer of 1 to 5, and when P2 is 2 or more, a plurality of X^{P1}'s may be the same or different. X^{P3} is a hydroxy group, a halogen atom, a carboxy group, a cyano group, a nitro group, an alkyl group, a fluorinated alkyl group, an alkoxycarbonyloxy group, an acyl group, an acyloxy group, or an alkoxy group. a1 is an integer of 0 to 3. When a1 is 2 or more, a plurality of X^{P3}'s are the same or different from each other. a2 is an integer of 1 to 3.

The base polymer may contain one type or a combination of two or more types of the structural units (I).

The lower limit of the content by percent of the structural unit (I) (a total content by percent when a plurality of types are contained) is preferably 10 mol %, more preferably 20 mol %, still more preferably 30 mol %, and particularly preferably 35 mol % based on all structural units constituting the base polymer. The upper limit of the content by percent is preferably 80 mol %, more preferably 70 mol %, still more preferably 60 mol %, and particularly preferably 55 mol %. When the content of the structural unit (I) is set to fall within the above range, the pattern-forming performance of the radiation-sensitive composition can further be improved.

### [Structural Unit (II)]

The structural unit (II) is a structural unit including at least one selected from the group consisting of a lactone structure, a cyclic carbonate structure and a sultone structure. The solubility of the base polymer into a developer can be adjusted by further introducing the structural unit (II). As a result, the radiation-sensitive composition can provide improved lithography properties such as the resolution. The adhesion between a resist pattern formed from the base polymer and a substrate can also be improved.

Examples of the structural unit (II) include structural units represented by the formulae (T-1) to (T-11).

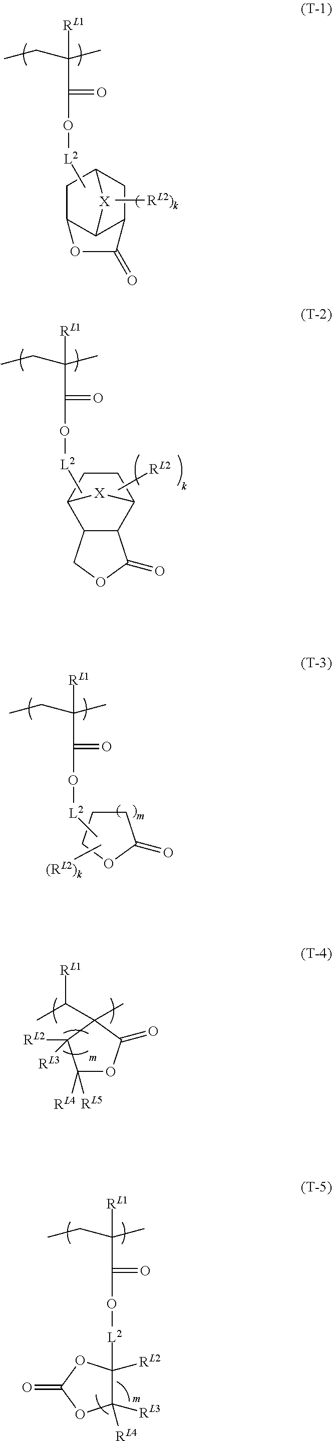

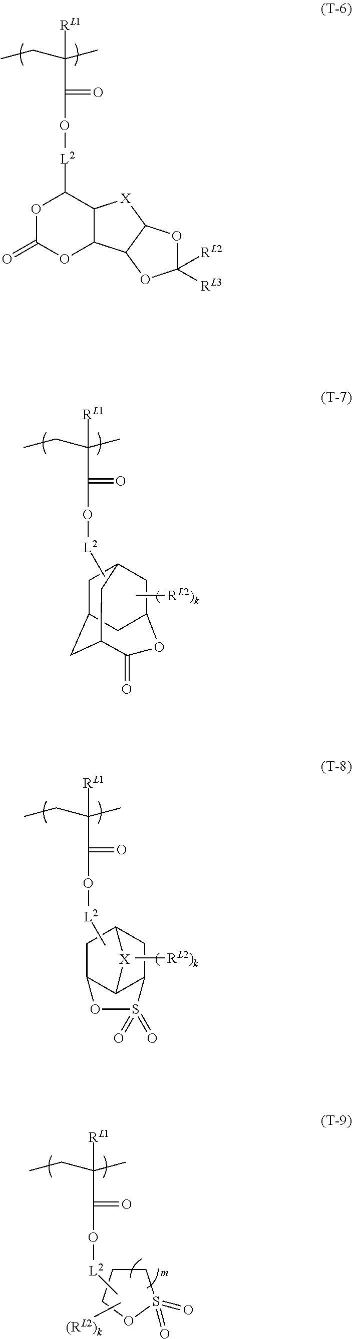

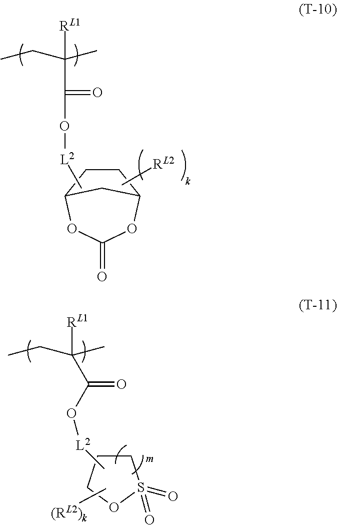

In the formulae, R^{L1} is a hydrogen atom, a fluorine atom, a methyl group, or a trifluoromethyl group; R^{L2} to R^{L5} are each independently a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group; R^{L4} and R^{L5} may be a divalent alicyclic group having 3 to 8 carbon atoms, which is obtained by combining R^{L4} and R^{L5} with the carbon atom to which they are bound. L² is a single bond, or a divalent linking group; X is an oxygen atom or a methylene group; k is an integer of 0 to 3; and m is an integer of 1 to 3.

Example of the divalent alicyclic group having 3 to 8 carbon atoms, which is composed of a combination of R^{L4} and R^{L5} with the carbon atom to which they are bound, includes the divalent alicyclic group having 3 to 8 carbon atoms in the divalent alicyclic group having 3 to 20 carbon atoms, which is composed of a combination of the chain hydrocarbon group or the alicyclic hydrocarbon group represented by R⁵³ and R⁵⁴ in the formula (2) with the carbon atom to which they are bound. One or more hydrogen atoms on the alicyclic group may be substituted with a hydroxy group.

Examples of the divalent linking group represented by L² as described above include a divalent straight or branched chain hydrocarbon group having 1 to 10 carbon atoms; a divalent alicyclic hydrocarbon group having 4 to 12 carbon atoms; and a group composed of one or more of the hydrocarbon group thereof and at least one group of —CO—, —O—, —NH— and —S—.

Among them, the structural unit (II) is preferably a group having a lactone structure, more preferably a group having a norbornane lactone structure, and further preferably a group derived from a norbornane lactone-yl (meth)acrylate.

The lower limit of the content by percent of the structural unit (II) is preferably 15 mol %, more preferably 20 mol %, and still more preferably 25 mol % based on all structural units constituting the base polymer. The upper limit of the content by percent is preferably 80 mol %, more preferably 70 mol %, and still more preferably 65 mol %. By adjusting the content by percent of the structural unit (II) within the ranges, the radiation-sensitive composition can provide improved lithography properties such as the resolution. The adhesion between the formed resist pattern and the substrate can also be improved.

### [Structural Unit (III)]

The base polymer optionally has another structural unit in addition to the structural units (I) and (II). Another structural unit includes a structural unit (III) containing a polar group (excluding those corresponding to the structural unit (II)). When the base polymer further has a structural unit (III), solubility in the developer can be adjusted. As a result, lithographic performance such as resolution of the radiation-sensitive composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (III) include structural units represented by the formulas.

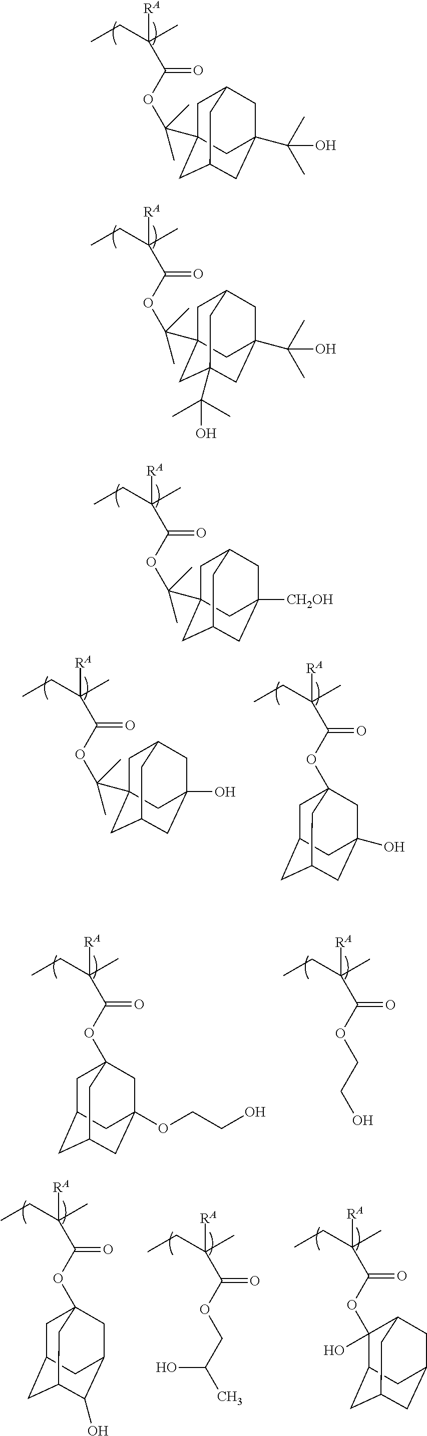

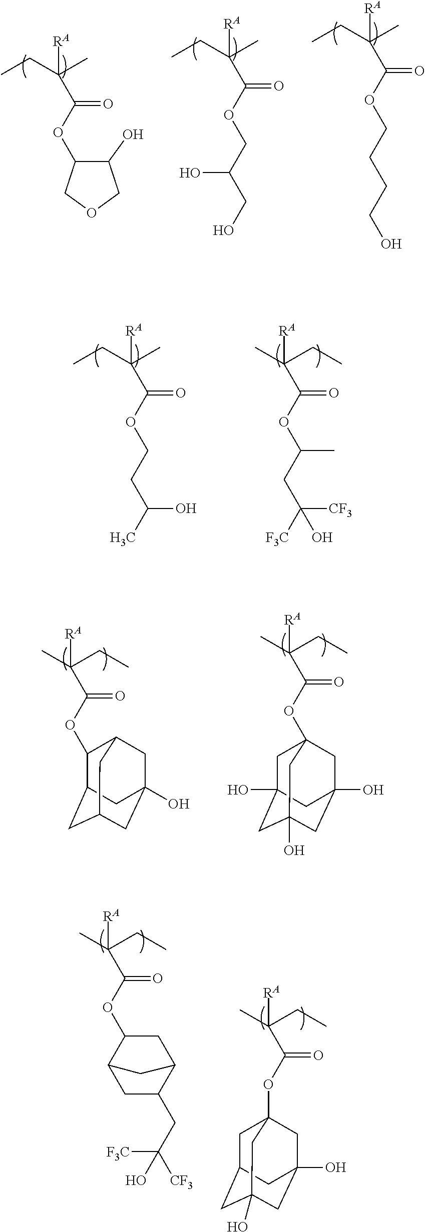

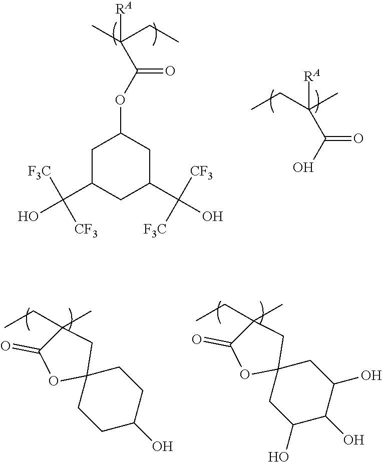

In the formulas, RA is a hydrogen atom, a fluorine atom, a methyl group, or a trifluoromethyl group.

When the base polymer has the structural unit (III) having a polar group, the lower limit of the content by percent of the structural unit (III) is preferably 2 mol %, more preferably 5 mol %, and still more preferably 8 mol % based on all structural units constituting the base polymer. The upper limit of the content by percent is preferably 40 mol %, more preferably 30 mol %, and still more preferably 25 mol %. When the content of the structural unit (III) is set to fall within the above range, the radiation-sensitive composition can provide further improved lithography properties such as the resolution.

### [Structural Unit (IV)]

The base polymer optionally has, as another structural unit, a structural unit derived from hydroxystyrene or a structural unit having a phenolic hydroxyl group (hereinafter, both are also collectively referred to as “structural unit (IV)”), in addition to the structural unit (III) having a polar group. The structural unit (IV) contributes to an improvement in etching resistance and an improvement in a difference in solubility of a developer (dissolution contrast) between an exposed part and a non-exposed part. In particular, the structural unit (IV) can be suitably applied to pattern formation using exposure with a radioactive ray having a wavelength of 50 nm or less, such as an electron beam or EUV. In this case, the polymer preferably has the structural unit (I) together with the structural unit (IV).

The structural unit derived from hydroxystyrene is represented by, for example, the formulas (4-1) to (4-2), and the structural unit containing a phenolic hydroxy group is represented by, for example, the formulas (4-3) to (4-4).

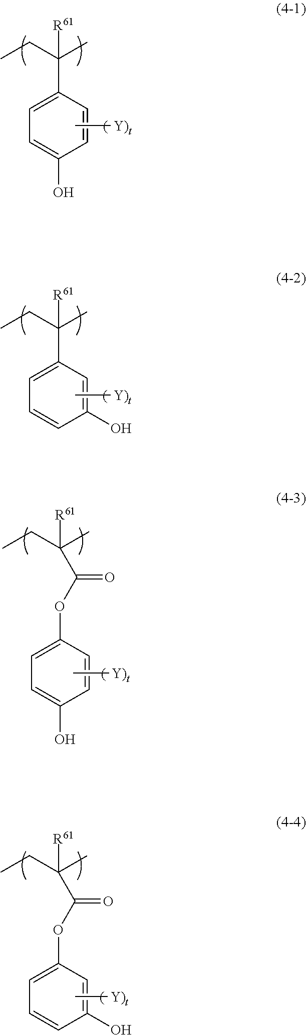

In the formulas (4-1) to (4-4), R⁶¹ is independently at each occurrence a hydrogen atom, a fluorine atom, a methyl group, or a trifluoromethyl group. Y is a halogen atom, a trifluoromethyl group, a cyano group, an alkyl or alkoxy group having 1 to 6 carbon atoms, or an acyl, acyloxy, or alkoxycarbonyl group having 2 to 7 carbon atoms. When there are a plurality of Y's, the plurality of Y's are the same or different from each other, t is an integer of 0 to 4.

When the structural unit (IV) is obtained, it is preferable to obtain the structural unit (IV) by polymerizing the corresponding monomer in a state where the phenolic hydroxy group is protected by a protecting group such as an alkali-dissociable group (e.g., an acyl group) during polymerization, and then deprotecting the polymerized product by hydrolysis.

In the case of a polymer for exposure to radiation having a wavelength of 50 nm or less, the lower limit of the content by percent of the structural unit (IV) is preferably 10 mol %, and more preferably 20 mol % based on all structural units constituting the polymer. The upper limit of the content by percent is preferably 70 mol %, and more preferably 60 mol %.

### [Other Structural Unit]

The base polymer may contain, as a structural unit other than the structural units listed above, a structural unit represented by the formula (6) and containing an alicyclic structure.

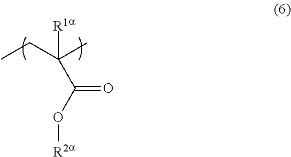

In the formula (6), R^{1α} represents a hydrogen atom, a fluorine atom, a methyl group, or a trifluoromethyl group, and R^{2α} represents a monovalent alicyclic hydrocarbon group having 3 to 20 carbon atoms.

In the formula (6), as the monovalent alicyclic hydrocarbon group having 3 to 20 carbon atoms represented by R^{2α}, the monovalent alicyclic hydrocarbon groups having 3 to 20 carbon atoms represented by R¹ and R² in the formula (1) can be suitably employed.

When the base polymer contains the structural unit having an alicyclic structure, the lower limit of the content by percent of the structural unit having an alicyclic structure is preferably 2 mol %, more preferably 5 mol %, and still more preferably 8 mol % based on all structural units constituting the base polymer. The upper limit of the content by percent is preferably 30 mol %, more preferably 20 mol %, and still more preferably 15 mol %.

### (Synthesis Method of Base Polymer)

For example, the base polymer can be synthesized by performing a polymerization reaction of each monomer for providing each structural unit with a radical polymerization initiator or the like in a suitable solvent.

Examples of the radical polymerization initiator include an azo-based radical initiator, including azobisisobutyronitrile (AIBN), 2,2′-azobis(4-methoxy-2,4-dimethylvaleronitrile), 2,2′-azobis(2-cyclopropylpropanenitrile), 2,2′-azobis(2,4-dimethylvaleronitrile), and dimethyl 2,2′-azobisisobutyrate; and peroxide-based radical initiator, including benzoyl peroxide, t-butyl hydroperoxide, and cumene hydroperoxide. Among them, AIBN or dimethyl 2,2′-azobisisobutyrate is preferred, and AIBN is more preferred. The radical initiator may be used alone, or two or more radical initiators may be used in combination.

Examples of the solvent used for the polymerization reaction include
alkanes including n-pentane, n-hexane, n-heptane, n-octane, n-nonane, and n-decane;
cycloalkanes including cyclohexane, cycloheptane, cyclooctane, decalin, and norbornane;
aromatic hydrocarbons including benzene, toluene, xylene, ethylbenzene, and cumene;
halogenated hydrocarbons including chlorobutanes, bromohexanes, dichloroethanes, hexamethylenedibromide, and chlorobenzenes;
saturated carboxylate esters, including ethyl acetate, n-butyl acetate, i-butyl acetate, and methyl propionate;
ketones including acetone, 2-butanone, 4-methyl-2-pentanone, 2-heptanone, and cyclohexanone;
lactones including γ-butyrolactone;
polyhydric alcohol partial ether carboxylates including propylene glycol monomethyl ether acetate;
ethers including tetrahydrofuran, propylene glycol monomethyl ether, dimethoxyethanes, and diethoxyethanes; and
alcohols including methanol, ethanol, 1-propanol, 2-propanol, 1-methoxy-2-propanol and 4-methyl-2-pentanol. The solvent used for the polymerization reaction may be used alone, or two or more solvents may be used in combination.

The reaction temperature of the polymerization reaction is typically from 40° C. to 150° C., and preferably from 50° C. to 120° C. The reaction time is typically from 1 hour to 48 hours, and preferably from 1 hour to 24 hours.

The molecular weight of the base polymer is not particularly limited, and the lower limit of the weight-average molecular weight (Mw) equivalent to polystyrene determined by gel permeation chromatography (GPC) is preferably 2,000, more preferably 3,000, still more preferably 4,000, and particularly preferably 4,500. The upper limit of the Mw is preferably 30,000, more preferably 20,000, still more preferably 12,000, and particularly preferably 10,000. By setting the Mw of the base polymer within the above range, it is possible to impart good heat resistance and developability to the resulting resist film.

For the base polymer, the ratio of Mw to the number average molecular weight (Mn) as determined by GPC relative to standard polystyrene (Mw/Mn) is typically 1 or more and 5 or less, preferably 1 or more and 3 or less, and more preferably 1 or more and 2 or less.

The Mw and Mn of the polymer in the specification are amounts measured by using Gel Permeation Chromatography (GPC) with the condition as described below.
GPC column: two G2000HXL, one G3000HXL, and one G4000HXL (all manufactured from Tosoh Corporation)
Column temperature: 40° C.
Eluting solvent: tetrahydrofuran
Flow rate: 1.0 mL/min
Sample concentration: 1.0% by mass
Sample injection amount: 100 μL
Detector: Differential Refractometer
Reference material: monodisperse polystyrene

The content by percent of the base polymer is preferably 60% by mass or more, more preferably 658 by mass or more, and still more preferably 70% by mass or more based on the total solid content of the radiation-sensitive composition.

### (Another Polymer)

The radiation-sensitive composition according to the present embodiment may contain, as another polymer, a polymer having higher content by mass of fluorine atoms than the above-described base polymer (hereinafter, also referred to as a “high fluorine-content polymer”). When the radiation-sensitive composition contains the high fluorine-content polymer, the high fluorine-content polymer can be localized in the surface layer of a resist film compared to the base polymer, which as a result makes it possible to enhance the water repellency of the surface of the resist film during immersion exposure or to perform surface modification of the resist film during EUV exposure or control of the distribution of the composition in the film.

The high fluorine-content polymer preferably has, for example, a structural unit represented by the formula (5) (hereinafter, also referred to as “structural unit (V)”), and may have the structural unit (I) or the structural unit (III) in the base polymer as necessary.

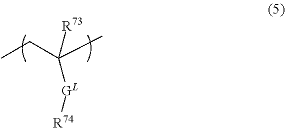

In the formula (5), R⁷³ is a hydrogen atom, a methyl group, or a trifluoromethyl group; GT is a single bond, an alkanediyl group having 1 to 5 carbon atoms, an oxygen atom, a sulfur atom, —COO—, —OCO—, —SO₂ONH—, —CONH—, —OCONH—, or a combination thereof; and R⁷⁴ is a monovalent fluorinated chain hydrocarbon group having 1 to 20 carbon atoms, or a monovalent fluorinated alicyclic hydrocarbon group having 3 to 20 carbon atoms.

As R⁷³ as described above, in terms of the copolymerizability of monomers resulting in the structural unit (V), a hydrogen atom or a methyl group is preferred, and a methyl group is more preferred.

As GL as described above, a combination of at least one of a single bond, —COO—, —OCO— and an alkanediyl group having 1 to 5 carbon atoms is preferable, and —COO— is more preferable from the viewpoint of the copolymerizability of a monomer that gives the structural unit (V).

Example of the monovalent fluorinated chain hydrocarbon group having 1 to 20 carbon atoms represented by R⁷⁴ as described above includes a group in which a part of or all of hydrogen atoms in the straight or branched chain alkyl group having 1 to 20 carbon atoms is/are substituted with a fluorine atom.

Example of the monovalent fluorinated alicyclic hydrocarbon group having 3 to 20 carbon atoms represented by R⁷⁴ as described above includes a group in which a part of or all of hydrogen atoms in the monocyclic or polycyclic hydrocarbon group having 3 to 20 carbon atoms is/are substituted with a fluorine atom.

The R⁷⁴ as described above is preferably a fluorinated chain hydrocarbon group, more preferably a fluorinated alkyl group, and further preferably 2,2,2-trifluoroethyl group, 2,2,3,3,3-pentafluoropropyl group, 1,1,1,3,3,3-hexafluoropropyl group and 5,5,5-trifluoro-1,1-diethylpentyl group.

When the high fluorine-content polymer has the structural unit (V), the lower limit of the content by percent of the structural unit (V) is preferably 40 mol %, more preferably 50 mol %, and still more preferably 55 mol % based on the total amount of all structural units constituting the high fluorine-content polymer. The upper limit of the content by percent is preferably 90 mol %, more preferably 80 mol %, and still more preferably 70 mol %. When the content of the structural unit (V) is set to fall within the above range, the content by mass of fluorine atoms of the high fluorine-content polymer can more appropriately be adjusted to further promote the localization of the high fluorine-content polymer in the surface layer of a resist film, as a result, the water repellency of the resist film during immersion exposure can be further improved.

The high fluorine-content polymer may have a fluorine atom-containing structural unit represented by the formula (f-2) (hereinafter, also referred to as a “structural unit (VI)”) in addition to or in place of the structural unit (V). When the high fluorine-content polymer has the structural unit (VI), solubility in an alkaline developing solution is improved, and therefore generation of development defects can be prevented.

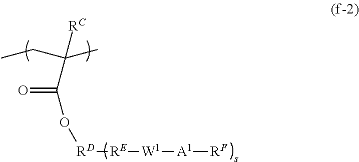

The structural unit (VI) is classified into two groups: a unit having an alkali soluble group (x); and a unit having a group (y) in which the solubility into the alkaline developing solution is increased by the dissociation by alkali (hereinafter, simply referred as an “alkali-dissociable group”). In both cases of (x) and (y), R^{C} in the formula (f-2) is a hydrogen atom, a fluorine atom, a methyl group, or a trifluoromethyl group; R^{D} is a single bond, a hydrocarbon group having 1 to 20 carbon atoms with the valency of (s+1), a structure in which an oxygen atom, a sulfur atom, —NR^{dd}—, a carbonyl group, —COO—, —OCO—, or —CONH— is connected to the terminal on R^{E} side of the hydrocarbon group, or a structure in which a part of hydrogen atoms in the hydrocarbon group is substituted with an organic group having a hetero atom; R^{dd} is a hydrogen atom, or a monovalent hydrocarbon group having 1 to 10 carbon atoms; and s is an integer of 1 to 3.

When the structural unit (VI) has the alkali soluble group (x), R^{F} is a hydrogen atom; A¹ is an oxygen atom, —COO—* or —SO₂O—*; * refers to a bond to R^{E}; W¹ is a single bond, a hydrocarbon group having 1 to 20 carbon atoms, or a divalent fluorinated hydrocarbon group. When A¹ is an oxygen atom, W¹ is a fluorinated hydrocarbon group having a fluorine atom or a fluoroalkyl group on the carbon atom connecting to A¹. R^{E} is a single bond, or a divalent organic group having 1 to 20 carbon atoms. When s is 2 or 3, a plurality of R^{E}, W¹, A¹ and R^{E} may be each identical or different. The affinity of the high fluorine-content polymer into the alkaline developing solution can be improved by including the structural unit (VI) having the alkali soluble group (x), and thereby prevent from generating the development defect. As the structural unit (VI) having the alkali soluble group (x), particularly preferred is a structural unit in which A¹ is an oxygen atom and W¹ is a 1,1,1,3,3,3-hexafluoro-2,2-methanediyl group.

When the structural unit (VI) has the alkali-dissociable group (y), R^{F} is a monovalent organic group having 1 to 30 carbon atoms; A¹ is an oxygen atom, —NR^{aa}—, —COO—*, —OCO—*, or —SO₂O—*; R^{aa} is a hydrogen atom, or a monovalent hydrocarbon group having 1 to 10 carbon atoms; * refers to a bond to R^{F}; W¹ is a single bond, or a divalent fluorinated hydrocarbon group having 1 to 20 carbon atoms; R^{E} is a single bond, or a divalent organic group having 1 to 20 carbon atoms. When A¹ is —COO—*, —OCO—* or —SO₂O—*, W¹ or R^{E} has a fluorine atom on the carbon atom connecting to A¹ or on the carbon atom adjacent to the carbon atom. When A¹ is an oxygen atom, W¹ and R^{E} are a single bond; R^{D} is a structure in which a carbonyl group is connected at the terminal on R^{E} side of the hydrocarbon group having 1 to 20 carbon atoms; and R^{F} is an organic group having a fluorine atom. When s is 2 or 3, a plurality of R^{E}, W¹, A¹ and R^{E} may be each identical or different. The surface of the resist film is changed from hydrophobic to hydrophilic in the alkaline developing step by including the structural unit (VI) having the alkali-dissociable group (y). As a result, the affinity of the high fluorine-content polymer into the alkaline developing solution can be significantly improved, and thereby prevent from generating the development defect more efficiently. As the structural unit (VI) having the alkali-dissociable group (y), particularly preferred is a structural unit in which A¹ is —COO—*, and R^{E} or W¹, or both is/are a group having a fluorine atom.

In terms of the copolymerizability of monomers resulting in the structural unit (VI), R^{C} is preferably a hydrogen atom or a methyl group, and more preferably a methyl group.

When R^{E} is a divalent organic group, R^{E} is preferably a group having a lactone structure, more preferably a group having a polycyclic lactone structure, and further preferably a group having a norbornane lactone structure.

When the high fluorine-content polymer has the structural unit (VI), the lower limit of the content by percent of the structural unit (VI) is preferably 40 mol, more preferably 50 mol %, and still more preferably 55 mol % based on the total amount of all structural units constituting the high fluorine-content polymer. The upper limit of the content by percent is preferably 95 mol %, more preferably 90 mol %, and still more preferably 85 mol %. When the content by percent of the structural unit (VI) is set to fall within the above range, water repellency of a resist film during immersion exposure can further be improved, and development defects can be suppressed.

### [Other Structural Unit]

The high fluorine-content polymer may contain a structural unit having an alicyclic structure represented by the formula (6) in addition to the structural unit (I) and the structural unit (III) in the base polymer as a structural unit other than the structural units listed above.

When the high fluorine-content polymer contains the structural unit (I) and the structural unit (III), the content by percent described for the base polymer can be suitably employed as the content by percent of each structural unit in the high fluorine-content polymer.

When the high fluorine-content polymer contains the structural unit having an alicyclic structure, the lower limit of the content by percent of the structural unit having an alicyclic structure is preferably 10 mol %, more preferably 20 mol %, and still more preferably 30 mol % based on all structural units constituting the high fluorine-content polymer. The upper limit of the content by percent is preferably 60 mol %, more preferably 50 mol %, and still more preferably 45 mol %.

The lower limit of the Mw of the high fluorine-content polymer is preferably 2,000, more preferably 3,000, still more preferably 4,000, and particularly preferably 5,000. The upper limit of the Mw is preferably 30,000, more preferably 20,000, still more preferably 10,000, particularly preferably 8,000.

The lower limit of the Mw/Mn of the high fluorine-content polymer is usually 1, and more preferably 1.1. In addition, the upper limit of the Mw/Mn is usually 5, preferably 3, and more preferably 2.

When the radiation-sensitive composition contains the high-fluorine-content polymer, the content of the high fluorine-containing polymer is preferably 0.5 parts by mass or more, more preferably 1 part by mass or more, still more preferably 1.5 parts by mass or more, and particularly preferably 2 parts by mass or more based on 100 parts by mass of the base polymer. The content of the high fluorine-containing polymer is preferably 15 parts by mass or less, more preferably 10 parts by mass or less, still more preferably 8 parts by mass or less, and particularly preferably 6 parts by mass or less.

When the content of the high fluorine-content polymer is set to fall within the above range, the high fluorine-content polymer can more effectively be localized in the surface layer of a resist film, which as a result makes it possible to further enhance the water repellency of the surface of the resist film during immersion exposure. The radiation-sensitive composition may contain one kind of high fluorine-content polymer or two or more kinds of high fluorine-content polymers.

### (Method for Synthesizing High Fluorine-Content Polymer)

The high fluorine-content polymer can be synthesized by a method similar to the above-described method for synthesizing a base polymer.

(Other Acid Diffusion Controlling Agent Other than the Onium Salt Compound (1) as the Acid Diffusion Controlling Agent)

The radiation-sensitive composition may contain other acid diffusion controlling agent other than the onium salt compound (1) as the acid diffusion controlling agent, as necessary. The acid diffusion controlling agent has the effect of controlling a phenomenon in which an acid generated from the onium salt compound (1) as a radiation-sensitive strong acid generator or from another radiation-sensitive strong acid generator by exposure diffuses in a resist film to prevent an undesired chemical reaction in an unexposed area. In addition, the storage stability of the resulting radiation-sensitive composition is improved. The acid diffusion controlling agent can further improve the resolution of the resist pattern and prevent from changing the line width of the resist pattern because of the variation of the pulling and placing time, i.e., the time from the exposure to the developing treatment, and therefore provide the radiation-sensitive composition having an improved process stability.

Examples of other acid diffusion controlling agent include a compound represented by the formula (7) (hereinafter, also referred as a “nitrogen-containing compound (I)”); a compound having two nitrogen atoms in one molecule (hereinafter, also referred as a “nitrogen-containing compound (II)”); a compound having three nitrogen atoms in one molecule (hereinafter, also referred as a “nitrogen-containing compound (III)”); a compound having an amide group; a urea compound; and a nitrogen-containing heterocyclic ring compound.

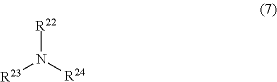

In the formula (7), R²², R²³ and R²⁴ are each independently a hydrogen atom, a substituted or unsubstituted alkyl group, a substituted or unsubstituted cycloalkyl group, a substituted or unsubstituted aryl group, or a substituted or unsubstituted aralkyl group.

Examples of the nitrogen-containing compound (I) include a monoalkylamine including n-hexylamine; a dialkylamine including di-n-butylamine; a trialkylamine including triethylamine; and an aromatic amine including aniline, 2,6-diisopropylaniline.

Examples of the nitrogen-containing compound (II) include ethylenediamine and N,N,N′,N′-tetramethylethylenediamine.

Examples of the nitrogen-containing compound (III) include a polyamine compound, including polyethyleneimine and polyallylamine; and a polymer including dimethylaminoethylacrylamide.

Examples of the amide-containing compound include formamide, N-methylformamide, N,N-dimethylformamide, acetamide, N-methylacetamide, N,N-dimethylacetamide, propionamide, benzamide, pyrrolidone, and N-methyl pyrrolidone.

Examples of the urea compound include urea, methylurea, 1,1-dimethylurea, 1,3-dimethylurea, 1,1,3,3-tetramethylurea, 1,3-diphenylurea, and tributylthiourea.

Examples of the nitrogen-containing heterocyclic ring compound include pyridines, including pyridine and 2-methylpyridine; morpholines, including N-propylmorpholine and N-(undecylcarbonyloxyethyl) morpholine; pyrazine, and pyrazole.

A compound having an acid-dissociable group may be used as the nitrogen-containing organic compound. Examples of the nitrogen-containing organic compound having an acid-dissociable group include N-t-butoxycarbonylpiperidine, N-t-butoxycarbonylimidazole, N-t-butoxycarbonylbenzimidazole, N-t-butoxycarbonyl-2-phenylbenzimidazole, N-t-amyloxycarbonyl-2-phenylbenzimidazole, N-(t-butoxycarbonyl)di-n-octylamine, N-(t-butoxycarbonyl) diethanolamine, N-(t-butoxycarbonyl) dicyclohexylamine, N-(t-butoxycarbonyl) diphenylamine, N-t-butoxycarbonyl-4-hydroxypiperidine, N-t-butoxycarbonyl-4-acetoxypiperidine, and N-t-amyloxycarbonyl-4-hydroxypiperidine.

As the acid diffusion controlling agent, a radiation-sensitive weak acid generator, which generates a weak acid by exposure to light, can be suitably used. The acid generated from the radiation-sensitive weak acid generator is a weak acid that does not induce dissociation of the acid-dissociable group under the conditions of dissociating the acid-dissociable group in the polymer. In the present specification, the “dissociation” of the acid-dissociable group refers to dissociation that occurs when post-exposure baking is performed at 110° C. for 60 seconds.

Examples of the radiation-sensitive weak acid generator include an onium salt compound that is decomposed by exposure to light to lose the acid diffusion controllability thereof. Examples of the radiation-sensitive weak acid generator include a sulfonium salt compound represented by formula (8-1) below, an iodonium salt compound represented by formula (8-2) below, and an ammonium salt compound represented by formula (8-5) below. In addition, a compound represented by the formula (8-3) containing a sulfonium cation and an anion in the same molecule and a compound represented by the formula (8-4) containing an iodonium cation and an anion in the same molecule are also included. It is noted that those corresponding to the onium salt compound (1) as an acid diffusion controlling agent are not included.

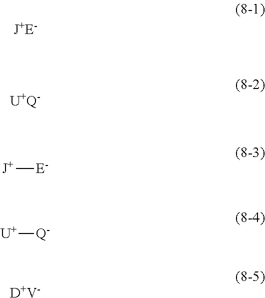

In the formulas (8-1) to (8-5), J⁺ is a sulfonium cation, U⁺ is an iodonium cation, and D⁺ is an ammonium cation. Examples of the sulfonium cation represented by J⁺ include sulfonium cations represented by the formulae (X-1) to (X-4). Examples of the iodonium cation represented by U⁺ include iodonium cations represented by the formulae (X-5) to (X-6). The ammonium cation represented by D⁺ is preferably represented by N⁺—(R⁵⁰) 4. A plurality of R⁵⁰'s are each independently a hydrogen atom or a monovalent hydrocarbon group. As the monovalent hydrocarbon group, monovalent hydrocarbon groups in the formula (1) can be suitably employed.

E⁻, Q⁻, and V⁻ are each independently an anion represented by OH⁻, R^{α}—COO—, or R^{α}—SO₃⁻. R^{α} is a single bond or a monovalent organic group having 1 to 30 carbon atoms (However, when the anion is represented by R^{α}—SO₃⁻, neither a fluorine atom nor a fluorinated hydrocarbon group is bonded to a carbon atom bonded to a sulfur atom in R^{α}.). Examples of the organic group include a monovalent hydrocarbon group having 1 to 20 carbon atoms, a group having a divalent hetero atom-containing group between carbon and carbon or at a carbon chain end of the hydrocarbon group, a group obtained by substituting some or all of hydrogen atoms of the hydrocarbon group with a monovalent hetero atom-containing group, or a combination thereof.

As the monovalent hydrocarbon group having 1 to 20 carbon atoms, monovalent hydrocarbon groups in the formula (1) can be suitably employed.

Examples of hetero atoms that constitute the divalent or monovalent hetero atom-containing group include an oxygen atom, a nitrogen atom, a sulfur atom, a phosphorus atom, a silicon atom, and a halogen atom. Examples of the halogen atom include a fluorine atom, a chlorine atom, a bromine atom, and an iodine atom.

As the divalent hetero atom-containing group, the divalent hetero atom-containing group in the formula (1) can be suitably employed.

Examples of the monovalent hetero atom-containing group include a hydroxy group, a sulfanyl group, a cyano group, a nitro group, and a halogen atom.

Examples of the onium salt compound include a compound represented by formula below.

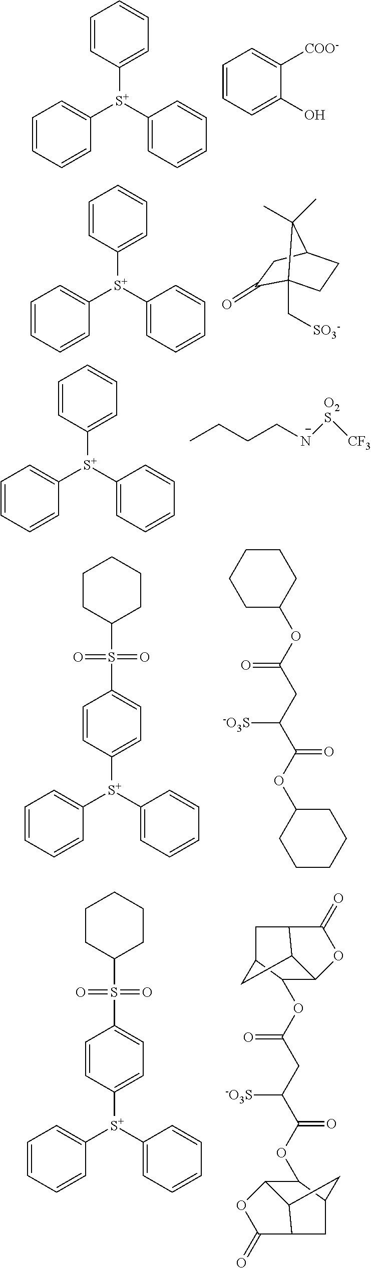

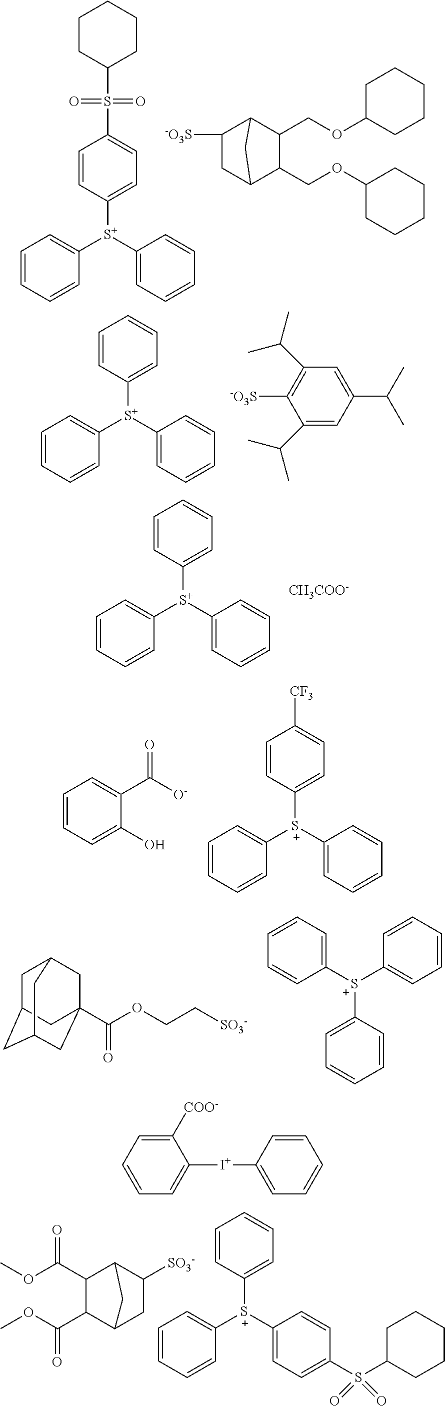

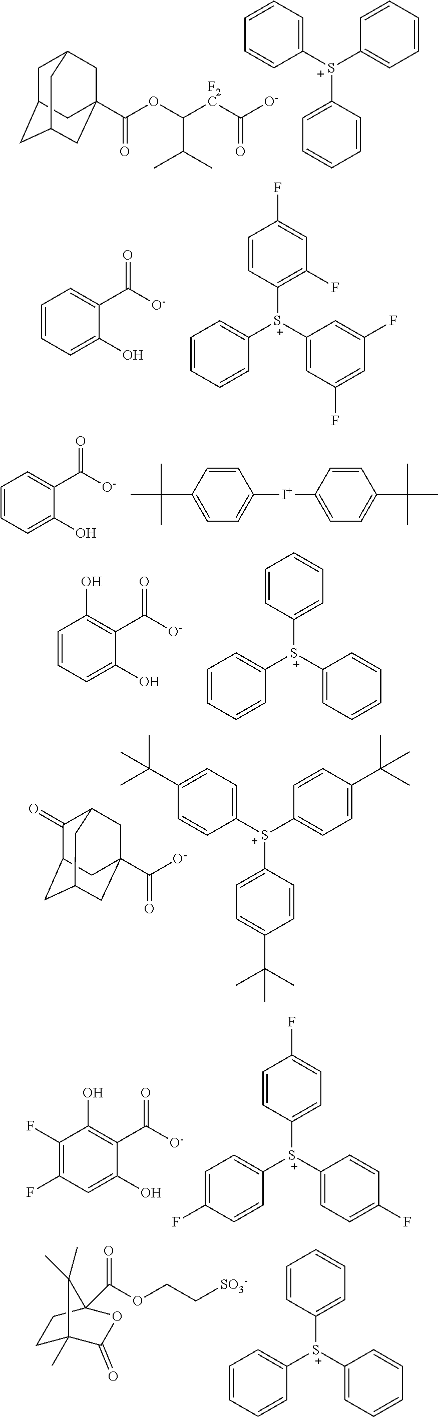

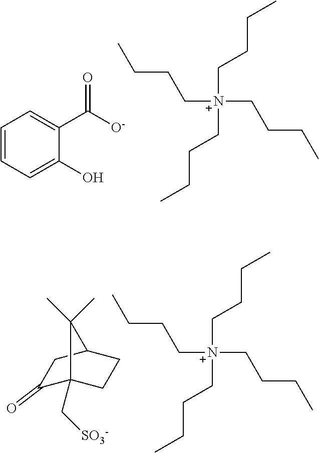

Among them, sulfonium salts are preferable as the radiation-sensitive weak acid generator, triarylsulfonium salts are more preferable, and triphenylsulfonium salicylate and triphenylsulfonium 10-camphorsulfonate are still more preferable.

The lower limit of the content of the acid diffusion controlling agent other than the onium salt compound (1) as an acid diffusion controlling agent is preferably 0.5 parts by mass, more preferably 1 part by mass, and still more preferably 2 parts by mass, based on 100 parts by mass of the polymer. The upper limit of the content is preferably 30 parts by mass, more preferably 20 parts by mass, and still more preferably 15 parts by mass.

When the content of the acid diffusion controlling agent is set within the above range, the lithographic performance of the radiation-sensitive composition can be further improved. The radiation-sensitive composition may include one type of the acid diffusion controlling agent, or two or more acid diffusion controlling agents in combination.

### (Solvent)

The radiation-sensitive composition according to the present embodiment contains a solvent. The solvent is not particularly limited as long as the solvent can dissolve or disperse at least an onium salt compound (1) and a polymer, and a radiation-sensitive acid generator contained as desired, and the like.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
a monoalcohol-based solvent having 1 to 18 carbon atoms, including iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
a polyhydric alcohol having 2 to 18 carbon atoms, including ethylene glycol, 1,2-propylene glycol, 2-methyl-2,4-pentanediol, 2,5-hexanediol, diethylene glycol, dipropylene glycol, triethylene glycol, and tripropylene glycol; and
a partially etherized polyhydric alcohol-based solvent in which a part of hydroxy groups in the polyhydric alcohol-based solvent is etherized.

Examples of the ether-based solvent include:
a dialkyl ether-based solvent, including diethyl ether, dipropyl ether, and dibutyl ether;
a cyclic ether-based solvent, including tetrahydrofuran and tetrahydropyran;
an ether-based solvent having an aromatic ring, including diphenylether and anisole (methyl phenyl ether); and
an etherized polyhydric alcohol-based solvent in which a hydroxy group in the polyhydric alcohol-based solvent is etherized.

Examples of the ketone-based solvent include:
a chain ketone-based solvent, including acetone, butanone, and methyl-iso-butyl ketone;
a cyclic ketone-based solvent, including cyclopentanone, cyclohexanone, and methylcyclohexanone; and
2,4-pentanedione, acetonylacetone, and acetophenone.

Examples of the amide-based solvent include:
a cyclic amide-based solvent, including N,N′-dimethyl imidazolidinone and N-methylpyrrolidone; and
a chain amide-based solvent, including N-methylformamide, N,N-dimethylformamide, N,N-diethylformamide, acetamide, N-methylacetamide, N,N-dimethylacetamide, and N-methylpropionamide.

Examples of the ester-based solvent include:
a monocarboxylate ester-based solvent, including n-butyl acetate and ethyl lactate;
a partially etherized polyhydric alcohol acetate-based solvent, including diethylene glycol mono-n-butyl ether acetate, propylene glycol monomethyl ether acetate, and dipropylene glycol monomethyl ether acetate;
a lactone-based solvent, including γ-butyrolactone and valerolactone;
a carbonate-based solvent, including diethyl carbonate, ethylene carbonate, and propylene carbonate; and
a polyhydric carboxylic acid diester-based solvent, including propylene glycol diacetate, methoxy triglycol acetate, diethyl oxalate, ethyl acetoacetate, and diethyl phthalate.

Examples of the hydrocarbon-based solvent include:
an aliphatic hydrocarbon-based solvent, including n-hexane, cyclohexane, and methylcyclohexane;
an aromatic hydrocarbon-based solvent, including benzene, toluene, di-iso-propylbenzene, and n-amylnaphthalene.

Among them, an ester-based solvent and an ether-based solvent are preferable, a polyhydric alcohol partial ether acetate-based solvent, a lactone-based solvent, a monocarboxylic acid ester-based solvent, and a ketone-based solvent are more preferable, and propylene glycol monomethyl ether acetate, γ-butyrolactone, ethyl lactate, cyclohexanone, and propylene glycol monomethyl ether are still more preferable. The radiation-sensitive composition may include one type of the solvent, or two or more types of the solvents in combination.

### (Other Optional Components)

The radiation-sensitive composition may contain other optional components other than the above-descried components. Examples of other optional components include a cross-linking agent, a localization enhancing agent, a surfactant, an alicyclic backbone-containing compound, and a sensitizer. These other optional components may be used singly or in combination of two or more of them.

### <Method for Preparing Radiation-Sensitive Composition>

The radiation-sensitive composition can be prepared by, for example, mixing the onium salt compound (1), the polymer, and, as necessary, the high fluorine-content polymer or the like, as well as the solvent in a prescribed ratio. The radiation-sensitive composition is preferably filtered through, for example, a filter having a pore diameter of about 0.05 μm to 0.40 μm after mixing. The solid matter concentration of the radiation-sensitive composition is usually 0.1 mass % to 50 mass %, preferably 0.5 mass % to 30 mass %, more preferably 1 mass % to 20 mass %.

### <Method for Forming Pattern>

A pattern forming method according to an embodiment of the present disclosure includes:
a step (1) of applying the radiation-sensitive composition directly or indirectly on a substrate to form a resist film (hereinafter, also referred to as a “resist film forming step”);
a step (2) of exposing the resist film (hereinafter, also referred to as an “exposure step”); and
a step (3) of developing the exposed resist film (hereinafter, also referred to as a “developing step”).

In accordance with this method for forming a resist pattern, a high-quality resist pattern can be formed because of the use of the radiation-sensitive composition described above capable of forming a resist film superior in sensitivity, LWR, DOF performance, pattern rectangularity, EL, CDU, and pattern circularity in an exposure step. Hereinbelow, each of the steps will be described.

### [Resist Film Forming Step]

In this step (the above mentioned step (1)), a resist film is formed with the radiation-sensitive composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 60° C. to 150° C., and preferably from 80° C. to 140° C. The duration of PB is typically from 5 seconds to 600 seconds, and preferably from 10 seconds to 300 seconds.

The lower limit of the thickness of the resist film to be formed is preferably 10 nm, more preferably 15 nm, and still more preferably 20 nm. The upper limit of the film thickness is preferably 500 nm, more preferably 400 nm, and still more preferably 300 nm. In particular, when a thick resist film is exposed to ArF excimer laser light in an exposure step described later, the lower limit of the film thickness may be 100 nm, may be 150 nm, or may be 200 nm.

When the immersion exposure is carried out, irrespective of presence of a water repellent polymer additive such as the high fluorine-content polymer in the radiation-sensitive composition, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing step (for example, see JP-A-2006-227632); or a developer-removable protective film that is removed during the development of the developing step (for example, see WO2005-069076 and WO2006-035790) may be used. In terms of the throughput, the developer-removable protective film is preferably used.

When the next step, the exposure step, is performed with radiation having a wavelength of 50 nm or less, it is preferable to use a polymer having the structural unit (I) and the structural unit (IV) as the base polymer in the composition.

### [Exposing Step]

In this step (the above mentioned step (2)), the resist film formed in the resist film forming step as the step (1) is exposed by irradiating with a radioactive ray through a photomask (optionally through an immersion medium such as water). Examples of the radioactive ray used for the exposure include visible ray, ultraviolet ray, far ultraviolet ray, extreme ultraviolet ray (EUV); an electromagnetic wave including X ray and γ ray; an electron beam; and a charged particle radiation such as α ray. Among them, far ultraviolet ray, an electron beam, or EUV is preferred. ArF excimer laser light (wavelength is 193 nm), KrF excimer laser light (wavelength is 248 nm), an electron beam, or EUV is more preferred. An electron beam or EUV having a wavelength of 50 nm or less which is identified as the next generation exposing technology is further preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the polymer by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 130° C. The duration of PEB is typically from 5 seconds to 600 seconds, and preferably from 10 seconds to 300 seconds.

### [Developing Step]

In this step (the above mentioned step (3)), the resist film exposed in the exposing step as the step (2) is developed. By this step, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive composition. Among them, an ether-based solvent, an ester-based solvent or a ketone-based solvent is preferred. As the ether-based solvent, a glycol ether-based solvent is preferable, and ethylene glycol monomethyl ether and propylene glycol monomethyl ether are more preferable. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably 80% by mass or more, more preferably 90% by mass or more, further preferably 95% by mass or more, and particularly preferably 99% by mass or more. Examples of the ingredient other than the organic solvent in the developer include water and silicone oil.

As described above, the developer may be either an alkaline developer or an organic solvent developer. The developer can be appropriately selected depending on whether the desired positive pattern or negative pattern is desired.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### <Radiation-Sensitive Acid Generator>

The radiation-sensitive acid generator according to the present embodiment is represented by the formula (1).

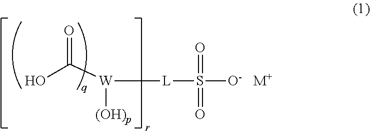

in the formula (1), W is an organic group having 3 to 40 carbon atoms and having at least one cyclic structure; L is a (r+1)-valent linking group, and r is an integer of 1 to 3; when r is 1, both p and q are integers of 1 to 3, and when r is 2 or 3, each of a plurality of p's and a plurality of q's is an integer of 0 to 3, provided that when r is 2 or 3, at least one of a plurality of p's is 1 or more and at least one of a plurality of q's is 1 or more; M⁺ is a monovalent onium cation.

As the onium salt compound represented by the formula (1), the onium salt compound (1) in the radiation-sensitive composition can be suitably employed.

### EXAMPLES

Hereinafter, the present invention will be specifically described with reference to Examples, but the present invention is not limited to these Examples. Methods for measuring various physical property values are shown below.

### [Weight-Average Molecular Weight (Mw) and Number-Average Molecular Weight (Mn)]

The Mw and Mn of a polymer were measured under the conditions described above. A degree of dispersion (Mw/Mn) was calculated from results of the measured Mw and Mn.

### [¹³C-NMR Analysis]

¹³C-NMR analysis of the polymer was performed using a nuclear magnetic resonance apparatus (“JNM-Delta 400” manufactured by JEOL Ltd.).

### <Synthesis of Polymer>

Monomers used for synthesis of polymers in Examples and Comparative Examples are shown below. In the following synthesis examples, unless otherwise specified, “parts by mass” means a value taken when the total mass of the monomers used is 100 parts by mass, and “mol %” means a value taken when the total number of moles of the monomers used is 100 mol %. 10

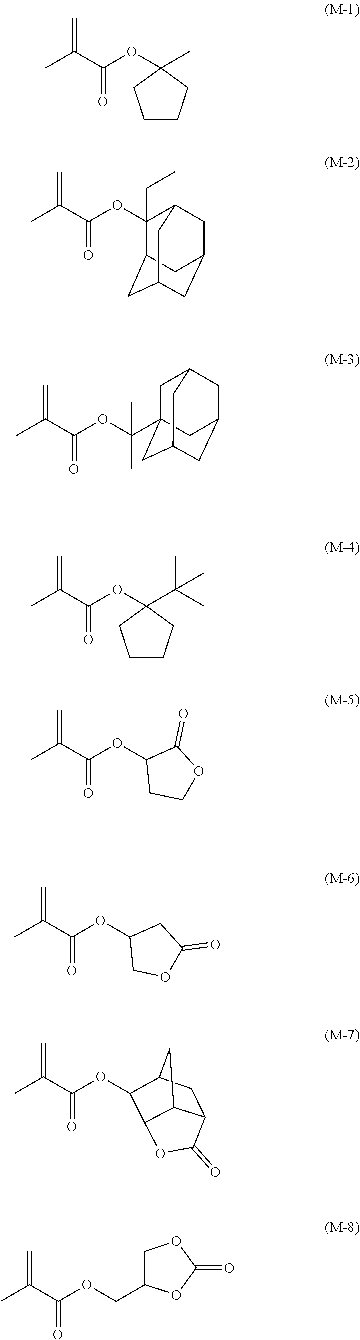

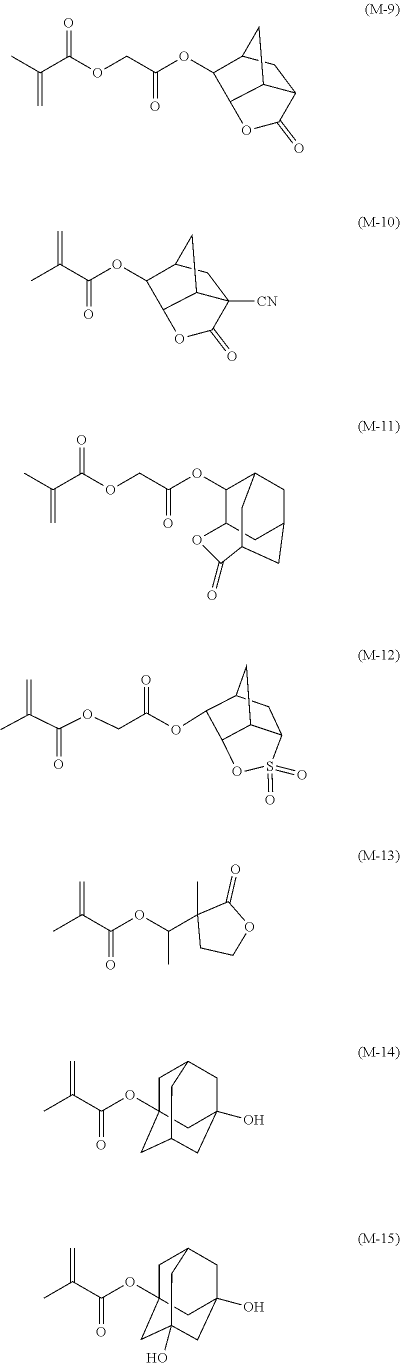

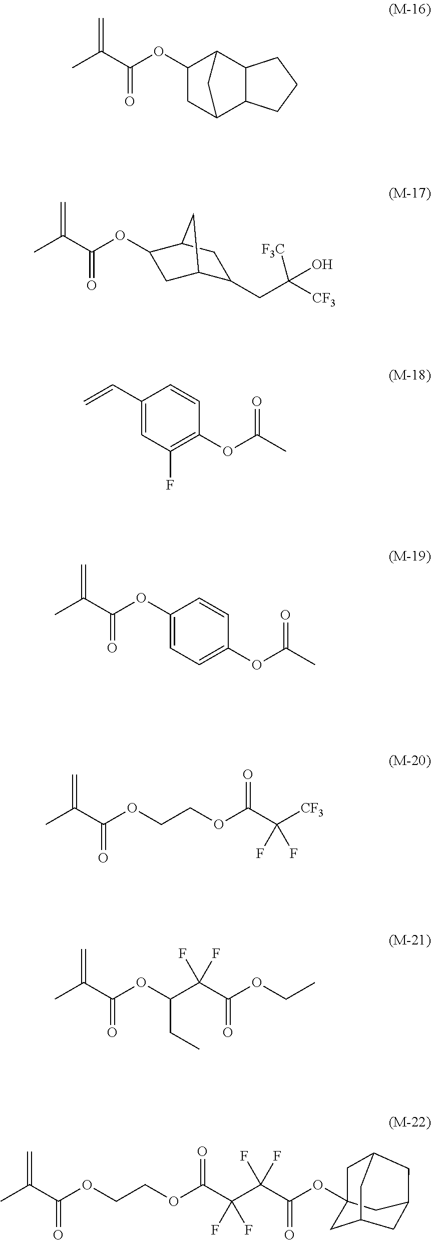

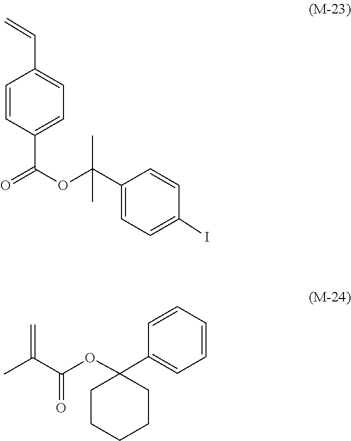 [SMILES: C=Cc1ccc(C(=O)OC(C)(C)c2ccc(I)cc2)cc1 ; C=C(C)C(=O)OC1(c2ccccc2)CCCCC1]

### Synthesis Example 1

### (Synthesis of Polymer (A-1))

A monomer (M-1), a monomer (M-2), a monomer (M-5), a monomer (M-10), and a monomer (M-14) were dissolved at a molar ratio of 40/10/20/20/10 (mol %) in 2-butanone (200 parts by mass), and AIBN (azobisisobutyronitrile) (5 mol % based on 100 mol % in total of the monomers used) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The polymerization solution cooled was poured into methanol (2,000 parts by mass), and a precipitated white powder was collected by filtration. The white powder separated by filtration was washed with methanol twice, then separated by filtration, and dried at 50° C. for 24 hours to obtain a white powdery polymer (A-1) (yield: 85%). The polymer (A-1) had an Mw of 7,100 and an Mw/Mn of 1.61. As a result of ¹³C-NMR analysis, the contents by percent of the structural units derived from (M-1), (M-2), (M-5), (M-10), and (M-14) were 40.3 mol %, 9.2 mol %, 20.5 mol %, 19.8 mol %, and 10.2 mol %, respectively.

### Synthesis Examples 2 to 11

### (Synthesis of Polymers (A-2) to (A-11))

Polymers (A-2) to (A-11) were synthesized in the same manner as in Synthesis Example 1 except that monomers of types and blending ratios shown in the following Table 1 were used. The content by percent (mol %) of each of the structural units and physical property values (Mw and Mw/Mn) of the resulting polymers are also shown in Table 1. In Table 1, “-” indicates that the corresponding monomer was not used (the same applies to Tables below).

TABLE 1

| | | Monomer that gives | | | Monomer that gives | | | Monomer that gives structural | | | | |
|---|---|---|---|---|---|---|---|---|---|---|---|---|
| | | structural unit (I) | | | structural unit (II) | | | unit (III) and the like | | | | |
| | | | | Content by | | | Content by | | | Content by | | |
| | | | | percent of | | | percent of | | | percent of | | |
| | | | Blending | structural | | Blending | structural | | Blending | structural | | |
| | Polymer | | ratio | unit | | ratio | unit | | ratio | unit | | |
| | [A] | Type | (mol %) | (mol %) | Type | (mol %) | (mol %) | Type | (mol %) | (mol %) | Mw | Mw/Mn |
| Synthesis | A-1 | M-1 | 40 | 40.3 | M-5 | 20 | 20.5 | M-14 | 10 | 10.2 | 7100 | 1.61 |
| Example 1 | | M-2 | 10 | 9.2 | M-10 | 20 | 19.8 | | | | | |
| Synthesis | A-2 | M-1 | 30 | 30.2 | M-9 | 50 | 50.6 | — | — | — | 7700 | 1.51 |
| Example 2 | | M-2 | 20 | 19.2 | | | | | | | | |
| Synthesis | A-3 | M-1 | 30 | 31.0 | M-11 | 50 | 49.4 | — | — | — | 7200 | 1.59 |
| Example 3 | | M-3 | 20 | 19.6 | | | | | | | | |
| Synthesis | A-4 | M-1 | 40 | 40.5 | M-12 | 50 | 49.2 | — | — | — | 6800 | 1.61 |
| Example 4 | | M-3 | 10 | 10.3 | | | | | | | | |
| Synthesis | A-5 | M-1 | 40 | 40.6 | M-13 | 50 | 51.3 | — | — | — | 6900 | 1.44 |
| Example 5 | | M-4 | 10 | 8.1 | | | | | | | | |
| Synthesis | A-6 | M-1 | 40 | 40.9 | M-6 | 20 | 20.5 | M-16 | 10 | 10.0 | 7500 | 1.51 |
| Example 6 | | M-4 | 10 | 8.3 | M-9 | 20 | 20.3 | | | | | |
| Synthesis | A-7 | M-1 | 50 | 50.2 | M-10 | 30 | 29.6 | M-14 | 20 | 20.2 | 7200 | 1.55 |
| Example 7 | | | | | | | | | | | | |
| Synthesis | A-8 | M-1 | 40 | 40.0 | M-7 | 20 | 20.5 | M-15 | 10 | 9.2 | 7100 | 1.62 |
| Example 8 | | M-3 | 10 | 10.1 | M-11 | 20 | 20.2 | | | | | |
| Synthesis | A-9 | M-1 | 50 | 50.3 | M-8 | 50 | 49.7 | — | — | — | 7000 | 1.51 |
| Example 9 | | | | | | | | | | | | |
| Synthesis | A-10 | M-1 | 40 | 40.2 | M-9 | 60 | 59.8 | — | — | — | 6700 | 1.50 |
| Example 10 | | | | | | | | | | | | |
| Synthesis | A-11 | M-2 | 40 | 39.4 | M-10 | 60 | 60.6 | — | — | — | 7500 | 1.49 |
| Example 11 | | | | | | | | | | | | |

### (Synthesis of Polymer (A-12))

Monomers (M-1) and (M-18) were dissolved at a molar ratio of 50/50 (mol %) in 1-methoxy-2 propanol (200 parts by mass), and AIBN (5 mol %) was added thereto as an initiator to prepare a monomer solution. 1-methoxy-2-propanol (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The cooled polymerization solution was poured into hexane (2,000 parts by mass), and a precipitated white powder was collected by filtration. The white powder separated by filtration was washed with hexane twice, then separated by filtration, and dissolved in 1-methoxy-2-propanol (300 parts by mass). Next, methanol (500 parts by mass), triethylamine (50 parts by mass) and ultrapure water (10 parts by mass) were added, and a hydrolysis reaction was performed at 70° C. for 6 hours with stirring. After the completion of the reaction, the remaining solvent was distilled off. The resulting solid was dissolved in acetone (100 parts by mass), and the solution was added dropwise to water (500 parts by mass) to solidify a polymer. The resulting solid was separated by filtration, and dried at 50° C. for 13 hours to obtain a white powdery polymer (A-12) (yield: 81%). The polymer (A-12) had an Mw of 5, 500 and an Mw/Mn of 1.62. As a result of ¹³C-NMR analysis, the contents by percent of the structural units derived from (M-1) and (M-18) were respectively 50.2 mol % and 49.8 mol %.

### Synthesis Examples 13 to 15

### (Synthesis of Polymers (A-13) to (A-15))

Polymers (A-13) to (A-15) were synthesized in the same manner as in Synthesis Example 12 except that monomers of types and blending ratios shown in the following Table 2 were used. In the monomers that give the structural unit (IV), all the alkali-dissociable groups had been hydrolyzed to phenolic hydroxy groups. The content by percent (mol %) of each of the structural units and physical property values (Mw and Mw/Mn) of the resulting polymers are also shown in Table 2.

TABLE 2

| | | Monomer that gives | | | Monomer that gives | | | Monomer that gives | | | | |
|---|---|---|---|---|---|---|---|---|---|---|---|---|
| | | structural unit (I) | | | structural unit (III) | | | structural unit (IV) | | | | |
| | | | | Content by | | | Content by | | | Content by | | |
| | | | | percent of | | | percent of | | | percent of | | |
| | | | Blending | structural | | Blending | structural | | Blending | structural | | |
| | Polymer | | ratio | unit | | ratio | unit | | ratio | unit | | |
| | [A] | Type | (mol %) | (mol %) | Type | (mol %) | (mol %) | Type | (mol %) | (mol %) | Mw | Mw/Mn |
| Synthesis | A-12 | M-1 | 50 | 50.2 | — | — | — | M-18 | 50 | 49.8 | 5500 | 1.62 |
| Example 12 | | | | | | | | | | | | |
| Synthesis | A-13 | M-23 | 50 | 46.6 | M-14 | 10 | 11.1 | M-19 | 40 | 42.3 | 5600 | 1.55 |
| Example 13 | | | | | | | | | | | | |
| Synthesis | A-14 | M-24 | 50 | 48.1 | M-17 | 20 | 21.3 | M-18 | 30 | 30.6 | 5100 | 1.59 |
| Example 14 | | | | | | | | | | | | |
| Synthesis | A-15 | M-4 | 55 | 53.2 | M-17 | 15 | 15.2 | M-19 | 30 | 31.6 | 6100 | 1.50 |
| Example 15 | | | | | | | | | | | | |

### Synthesis Example 16

### [Synthesis of High Fluorine-Containing Polymer (F-1)]

Monomers (M-1), (M-15) and (M-20) were dissolved at a molar ratio of 20/10/70 (mol %) in 2-butanone (200 parts by mass), and AIBN (4 mol %) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The solvent was replaced with acetonitrile (400 parts by mass). Hexane (100 parts by mass) was then added, followed by stirring, and an acetonitrile layer was collected. The operation was repeated three times. By replacing the solvent with propylene glycol monomethyl ether acetate, a solution of a high fluorine-containing polymer (F-1) was obtained (yield: 78%). The high fluorine-content polymer (F-1) had an Mw of 6,200 and an Mw/Mn of 1.77. As a result of ¹³C-NMR analysis, the contents by percent of the structural units derived from (M-1), (M-15) and (M-20) were 20.2 mol %, 9.5 mol % and 70.3 mol %, respectively.

### Synthesis Examples 17 to 20

### (Synthesis of High Fluorine-Containing Polymers (F-2) to (F-5)

High fluorine-containing polymers (F-2) to (F-5) were synthesized in the same manner as in Synthesis Example 16 except that monomers of types and blending ratios shown in Table 3 were used. The content by percent (mol %) of each of the structural units and physical property values (Mw and Mw/Mn) of the resulting high fluorine-containing polymers are also shown in Table 3.

TABLE 3
Monomer that gives structural Monomer that gives structural

| | | Monomer that gives | | | | | | |
|---|---|---|---|---|---|---|---|---|
| | | structural unit | | | Monomer that gives | | | |
| | | (V) or (VI) | | | structural unit (I) | | | |
| | High | | | Content by | | | Content by | |
| | fluorine- | | | percent of | | | percent of | |
| | content | | Blending | structural | | Blending | structural | Monomer that gives |
| | polymer | | ratio | unit | | ratio | unit | structural unit (III) |
| | [F] | Type | (mol %) | (mol %) | Type | (mol %) | (mol %) | Type |
| Synthesis | F-1 | M-20 | 70 | 70.3 | M-1 | 20 | 20.2 | M-15 |
| Example 16 | | | | | | | | |
| Synthesis | F-2 | M-21 | 80 | 80.9 | M-4 | 20 | 19.1 | — |
| Example 17 | | | | | | | | |
| Synthesis | F-3 | M-22 | 60 | 62.3 | — | — | — | — |
| Example 18 | | | | | | | | |
| Synthesis | F-4 | M-22 | 60 | 60.2 | M-2 | 20 | 19.4 | M-14 |
| Example 19 | | | | | | | | |
| Synthesis | F-5 | M-20 | 60 | 60.0 | M-3 | 10 | 10.1 | M-17 |
| Example 20 | | | | | | | | |

| | Monomer that gives | | Monomer that gives | | | | |
|---|---|---|---|---|---|---|---|
| | structural unit (III) | | other structural unit | | | | |
| | | Content by | | | Content by | | |
| | | percent of | | | percent of | | |
| | Blending | structural | | Blending | structural | | |
| | ratio | unit | | ratio | unit | | |
| | (mol %) | (mol %) | Type | (mol %) | (mol %) | Mw | Mw/Mn |
| Synthesis | 10 | 9.5 | — | — | — | 6200 | 1.77 |
| Example 16 | | | | | | | |
| Synthesis | — | — | — | — | — | 7100 | 1.82 |
| Example 17 | | | | | | | |
| Synthesis | — | — | M-16 | 40 | 37.7 | 6900 | 1.91 |
| Example 18 | | | | | | | |
| Synthesis | 20 | 20.4 | — | — | — | 7300 | 1.88 |
| Example 19 | | | | | | | |
| Synthesis | 30 | 29.9 | — | — | — | 6700 | 1.87 |
| Example 20 | | | | | | | |

### [B] Synthesis of Onium Salt Compound (1)

### Example B1

### (Synthesis of Compound (B-1))

A compound (B-1) was synthesized according to a synthesis scheme below.

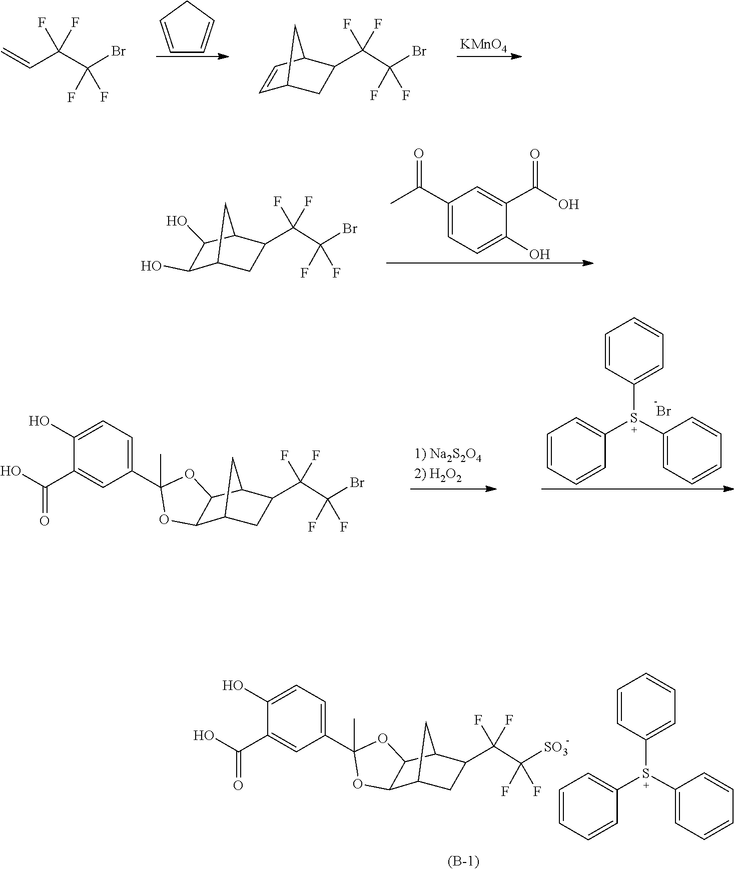

In a reaction vessel, 20.0 mmol of cyclopentadiene and 50 g of methylene chloride were added to 20.0 mmol of 4-bromo-3,3,4,4-tetrafluoro-1-butene, and the mixture was stirred at room temperature for 3 hours. Thereafter, water was added to dilute the mixture, followed by addition of methylene chloride and extraction, and then the organic layer was separated. The resulting organic layer was washed with a saturated aqueous sodium chloride solution and then with water. After drying over sodium sulfate, the solvent was distilled off, and the residue was purified by column chromatography, affording an olefin form in a good yield.

To the olefin form were added 40.0 mmol of potassium permanganate and 50 g of acetonitrile, and the mixture was stirred at 50° C. for 10 hours. Thereafter, a saturated aqueous sodium thiosulfate solution was added to stop the reaction, followed by addition of ethyl acetate and extraction, and then the organic layer was separated. The resulting organic layer was washed with a saturated aqueous sodium chloride solution and then with water. After drying over sodium sulfate, the solvent was distilled off, and the residue was purified by column chromatography, affording a diol form in a good yield.

To the diol form were added 20.0 mmol of 5-acetylsalicylic acid, 2.00 mmol of sulfuric acid, and 50 g of dichloromethane, and the mixture stirred at room temperature for 24 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, a solvent was distilled off, and the residue was purified by column chromatography, affording an acetal form in a good yield.

A mixed liquid of acetonitrile and water (1:1 (mass ratio)) was added to the acetal form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. The resulting organic layer was dried over sodium sulfate, the solvent was then distilled off, and the residue was purified by column chromatography, affording a compound (B-1) represented by the formula (B-1) in a good yield.

### Examples B2 to B9

### (Synthesis of Compounds (B-2) to (B-9))

Onium salt compounds (1) represented by formulas (B-2) to (B-9) below were synthesized in the same manner as in Example B1 except that the raw materials and the precursor were appropriately changed.

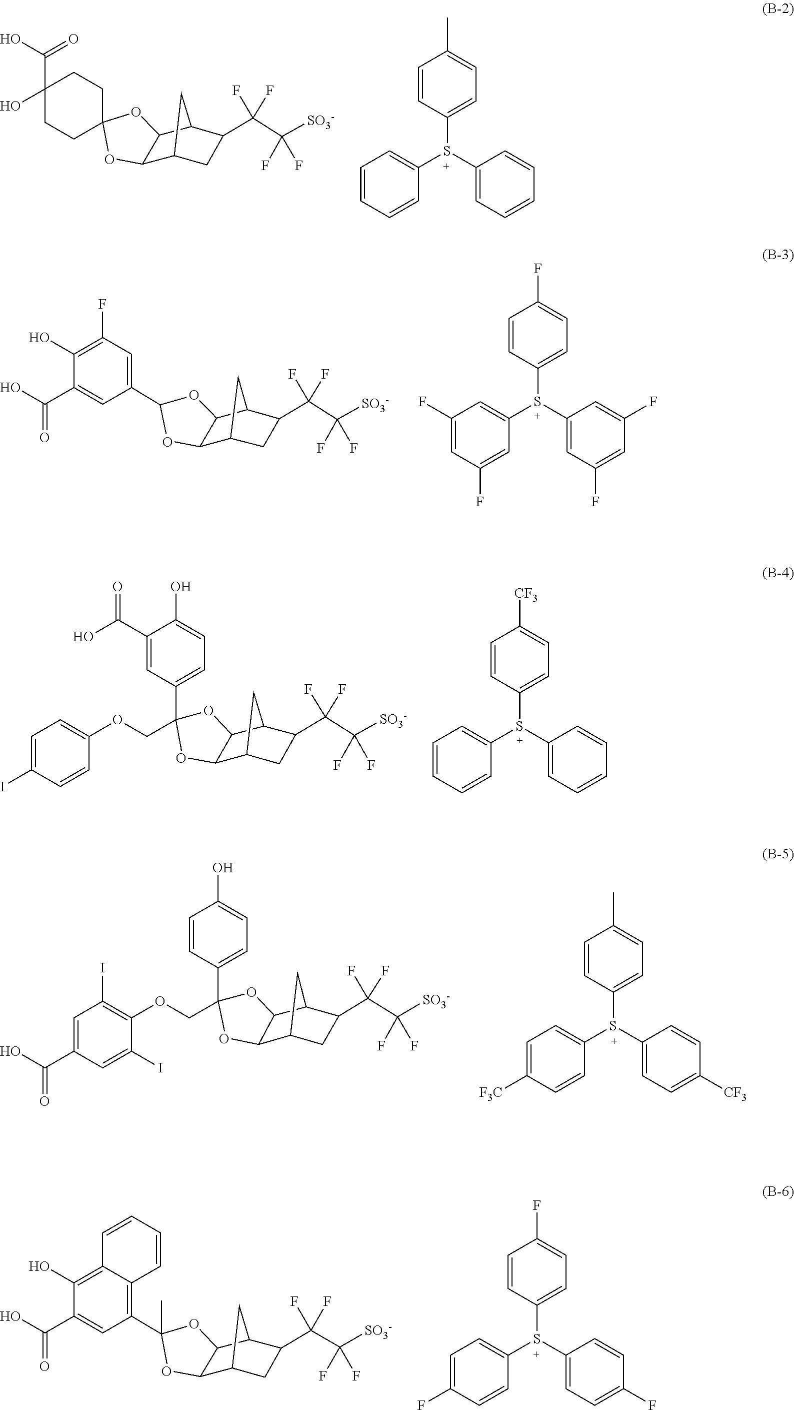

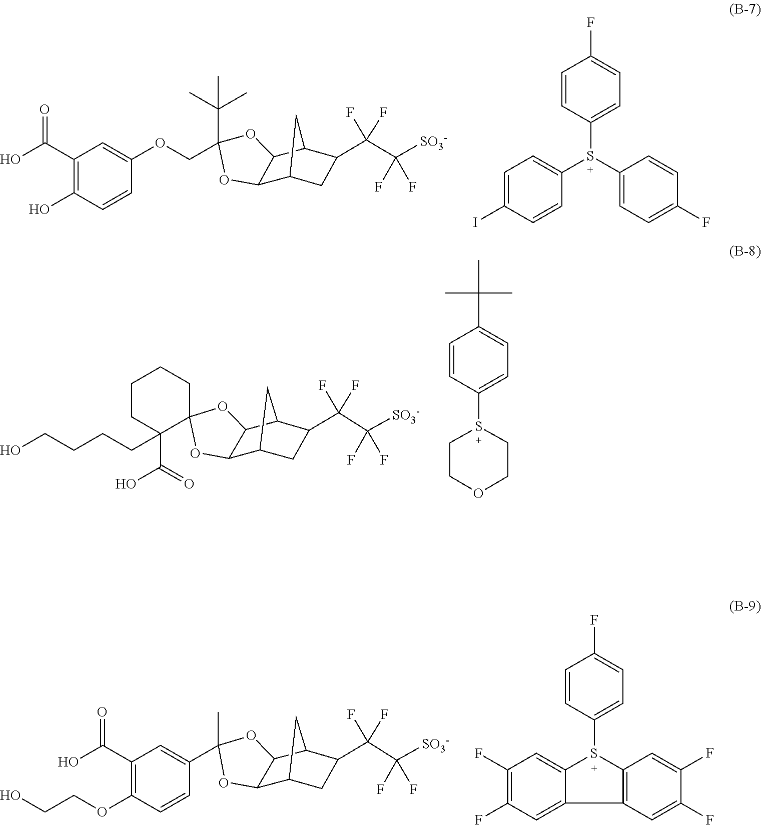

### Example B10

### (Synthesis of Compound (B-10))

A compound (B-10) was synthesized according to a synthesis scheme below.

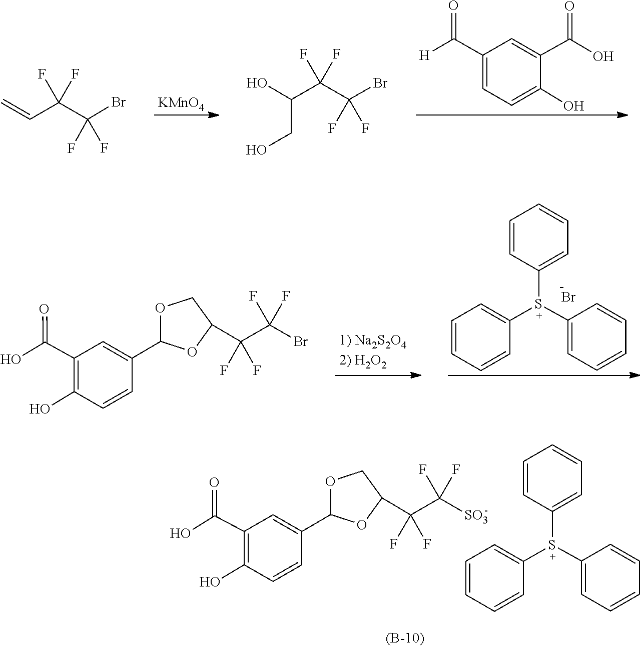

To a reaction vessel were added 20.0 mmol of 4-bromo-3,3,4,4-tetrafluoro-1-butene, 40.0 mmol of potassium permanganate, and 50 g of acetonitrile, and the mixture was stirred at 50° C. for 10 hours. Thereafter, a saturated aqueous sodium thiosulfate solution was added to stop the reaction, followed by addition of ethyl acetate and extraction, and then the organic layer was separated. The resulting organic layer was washed with a saturated aqueous sodium chloride solution and then with water. After drying over sodium sulfate, the solvent was distilled off, and the residue was purified by column chromatography, affording a diol form in a good yield.

To the diol form were added 20.0 mmol of 5-formylsalicylic acid, 2.00 mmol of sulfuric acid, and 50 g of dichloromethane, and the mixture stirred at room temperature for 24 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, a solvent was distilled off, and the residue was purified by column chromatography, affording an acetal form in a good yield.

A mixed liquid of acetonitrile and water (1:1 (mass ratio)) was added to the acetal form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. The resulting organic layer was dried over sodium sulfate, the solvent was then distilled off, and the residue was purified by column chromatography, affording a compound (B-10) represented by the formula (B-10) in a good yield.

### Example B11

### (Synthesis of Compound (B-11))

A compound (B-11) was synthesized according to a synthesis scheme below.

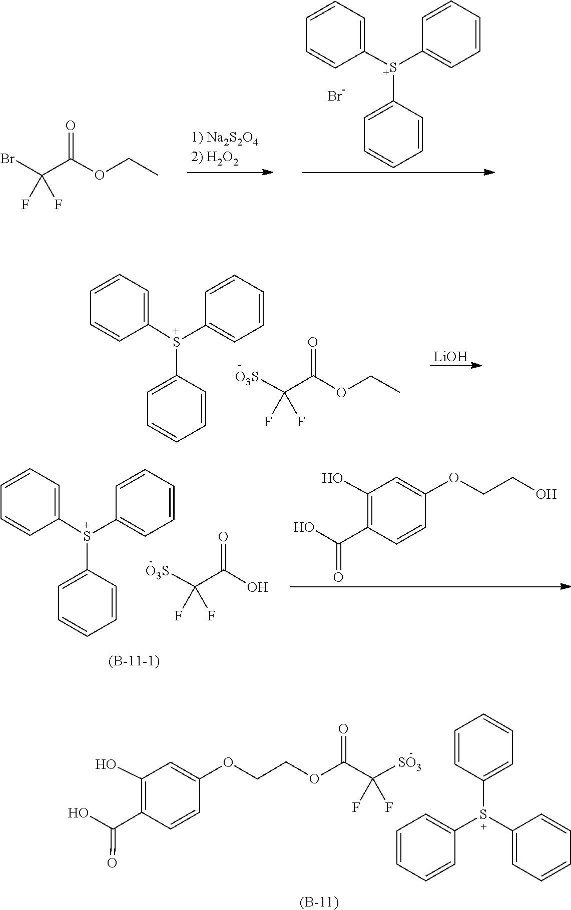

A mixed liquid of acetonitrile and water (1:1 (mass ratio)) was added to 20.0 mmol of ethyl bromodifluoroacetate to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. After drying the resulting organic layer over sodium sulfate, the solvent was distilled off, and purification was performed by column chromatography, affording an onium salt form in good yield.

A mixed liquid of methanol and water (1:1 (mass ratio)) was added to the onium salt form to form a 1 M solution. Then, 20.0 mmol of lithium hydroxide was added, and the resulting mixture was reacted at room temperature for 2 hours. Thereafter, 2 M hydrochloric acid was added to stop the reaction, followed by addition methylene chloride and extraction, and then the organic layer was separated. The resulting organic layer was dried over sodium sulfate, the solvent was then distilled off, and the residue was purified by column chromatography, affording a compound (B-11-1) represented by the formula (B-11-1) in a good yield.

To the compound (B-11-1) were added 20.0 mmol of 4-(2-hydroxyethoxy) salicylic acid, 20.0 mmol of dicyclohexylcarbodiimide, 2.0 mmol of 4-dimethylaminopyridine and 50 g of methylene chloride, and the mixture was stirred at room temperature for 3 hours. Thereafter, 1 M hydrochloric acid was added to dilute the mixture, followed by addition of methylene chloride and extraction, and then the organic layer was separated. The resulting organic layer was washed with a saturated aqueous sodium chloride solution and then with water. After drying over sodium sulfate, the solvent was distilled off, and the residue was purified by column chromatography, affording a compound (B-11) represented by the formula (B-11) in a medium yield.

### Examples B12 to B13

### (Synthesis of Compounds (B-12) to (B-13))

Onium salt compounds (1) represented by formulas (B-12) to (B-13) below were synthesized in the same manner as in Example B11 except that the raw materials and the precursor were appropriately changed.

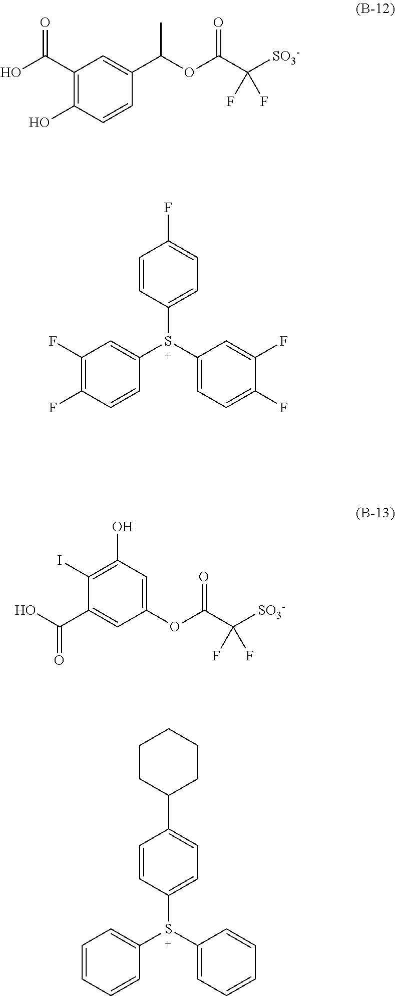

### Example B14

### (Synthesis of Compound (B-14))

A compound (B-14) was synthesized according to a synthesis scheme below.

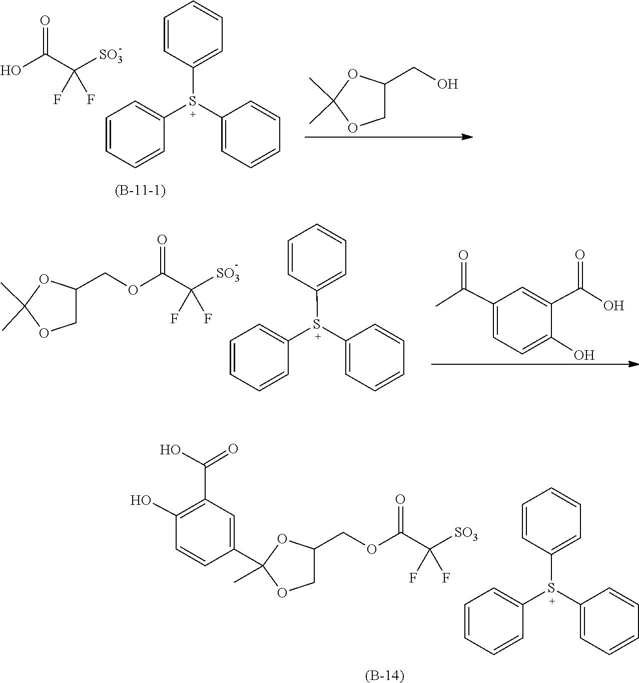

To a reaction vessel were added 20.0 mmol of the compound (B-11-1), 20.0 mmol of 1,2-isopropylidene glycol, 30.0 mmol of dicyclohexylcarbodiimide and 50 g of methylene chloride, and the mixture was stirred at room temperature for 10 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, the solvent was distilled off, and the residue was purified by column chromatography, affording an ester form in good yield.

To the ester form were added 20.0 mmol of 5-acetylsalicylic acid, 2.00 mmol of sulfuric acid, and 50 g of dichloroethane, and the mixture stirred at 70° C. for 24 hours. Thereafter, water was added to dilute the mixture, followed by addition of dichloromethane and extraction, and then the organic layer was separated. The resulting organic layer was washed with a saturated aqueous sodium chloride solution and then with water. After drying over sodium sulfate, the solvent was distilled off, and the residue was purified by column chromatography, affording a compound (B-14) represented by the formula (B-14) in a good yield.

### Example B15

### (Synthesis of Compound (B-15))

A compound (B-15) was synthesized according to a synthesis scheme below.

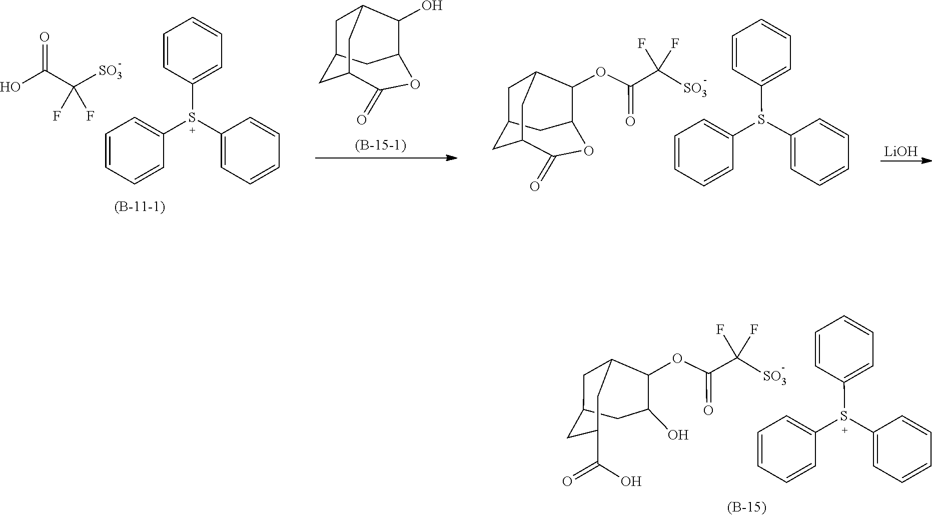

To a reaction vessel were added 20.0 mmol of the compound (B-11-1), 20.0 mmol of the compound (B-15-1), 3.0 mmol of p-toluenesulfonic acid monohydrate and 50 g of toluene, and the mixture was stirred at 100° C. for 10 hours. Thereafter, a saturated aqueous sodium bicarbonate solution was added to stop the reaction, followed by addition of dichloromethane and extraction, and then the organic layer was separated. The resulting organic layer was washed with a saturated aqueous sodium chloride solution and then with water. After drying over sodium sulfate, the solvent was distilled off, and the residue was purified by column chromatography, affording an ester form in good yield.

A mixed liquid of acetonitrile and water (1:1 (mass ratio)) was added to the ester form to form a 1 M solution. Then, 20.0 mmol of lithium hydroxide was added, and the mixture was reacted at room temperature for 2 hours. Thereafter, 2 M hydrochloric acid was added to stop the reaction, followed by addition methylene chloride and extraction, and then the organic layer was separated. The resulting organic layer was dried over sodium sulfate, the solvent was then distilled off, and the residue was purified by column chromatography, affording a compound (B-15) represented by the formula (B-15) in a medium yield.

### Example B16

### (Synthesis of Compound (B-16))

A compound (B-16) was synthesized according to a synthesis scheme below.

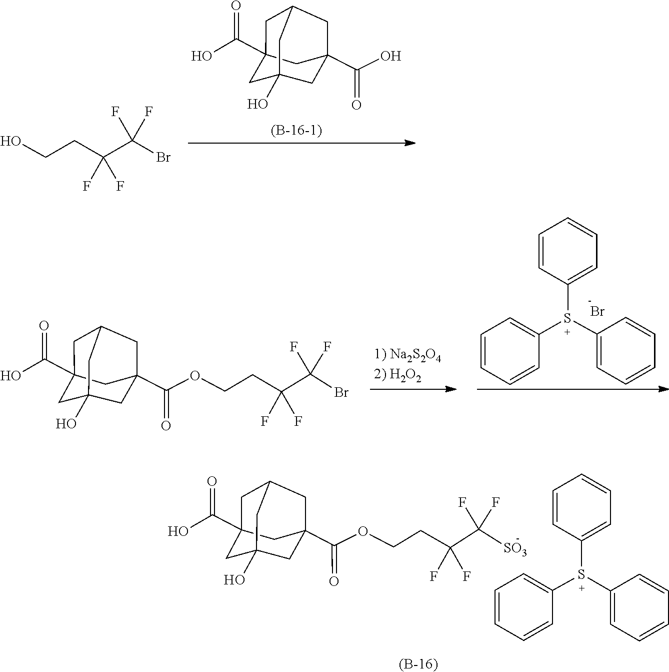

To a reaction vessel were added 20.0 mmol of 4-bromo-3,3,4,4-tetrafluorobutan-1-ol, 20.0 mmol of the compound (B-16-1), 20.0 mmol of dicyclohexylcarbodiimide and 50 g of acetonitrile, and the mixture was stirred at room temperature for 10 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, the solvent was distilled off, and the residue was purified by column chromatography, affording an ester form in good yield.

A mixed liquid of acetonitrile and water (1:1 (mass ratio)) was added to the ester form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. The resulting organic layer was dried over sodium sulfate, the solvent was then distilled off, and the residue was purified by column chromatography, affording a compound (B-16) represented by the formula (B-16) in a good yield.

### Examples B17 to B20

### (Synthesis of Compounds (B-17) to (B-20))

Onium salt compounds (1) represented by formulas (B-17) to (B-20) below were synthesized in the same manner as in Example B16 except that the raw materials and the precursor were appropriately changed.

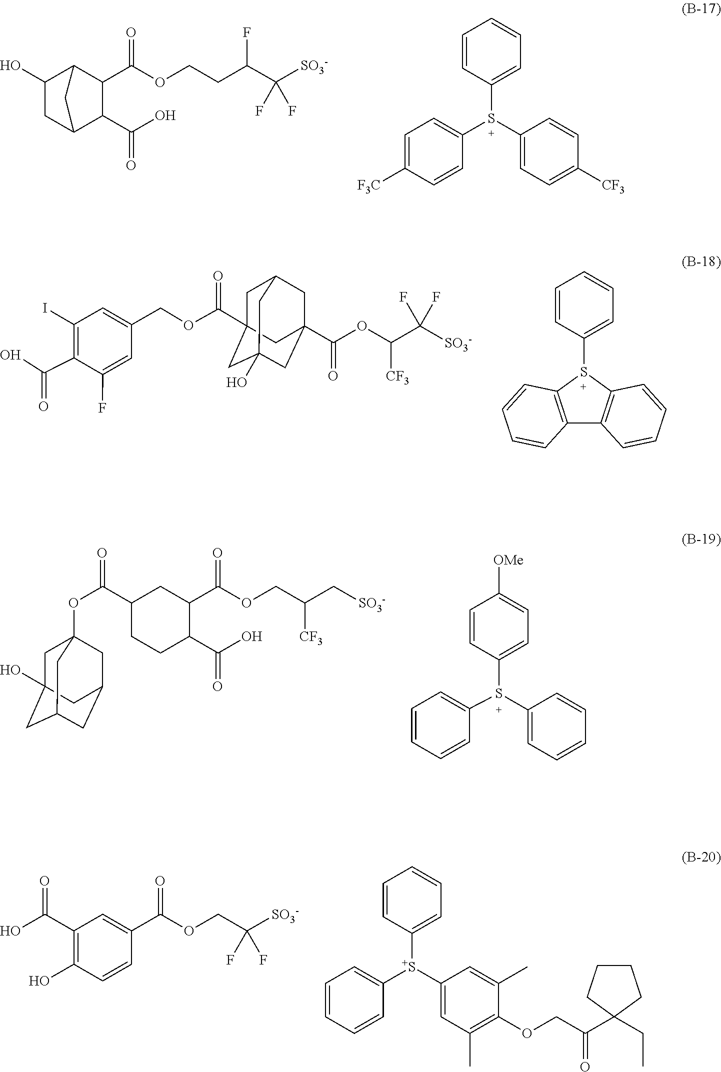

<Synthesis of Onium Salt Compounds (Radiation-Sensitive Acid Generators) Other than Above>

The following compounds were used as components other than the synthesized components.

[Onium Salt Compound (Radiation-Sensitive Acid Generator) Other than Onium Salt Compounds (B-1) to (B-20)]

b-1 to b-8: Compounds represented by formulas (b-1) to (b-8) below (hereinafter, may be described as “compound (b-1)” to “compound (b-8)”, respectively).

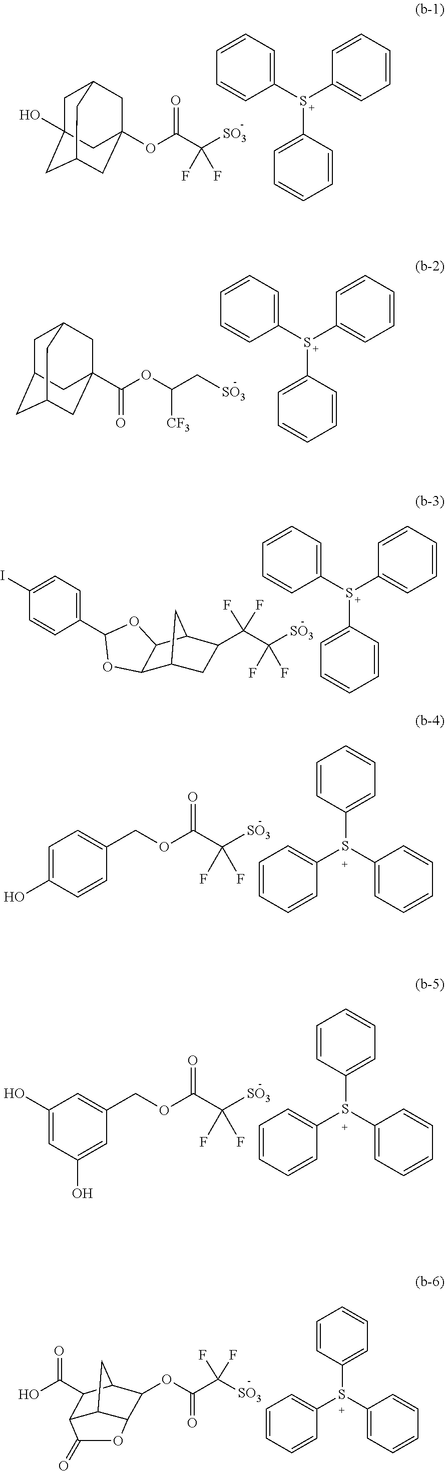

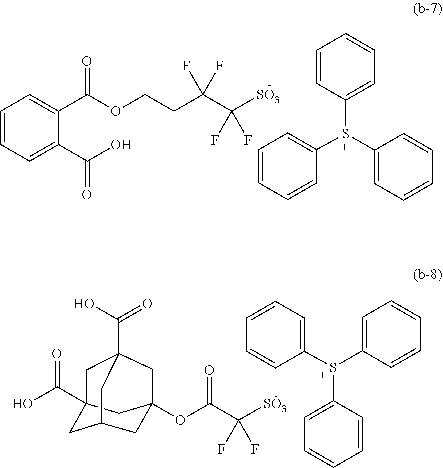

### Synthesis of Acid Diffusion Controlling Agent D

### Example D1

(Synthesis of compound (D-1))

A compound (D-1) was synthesized according to a synthesis scheme below.

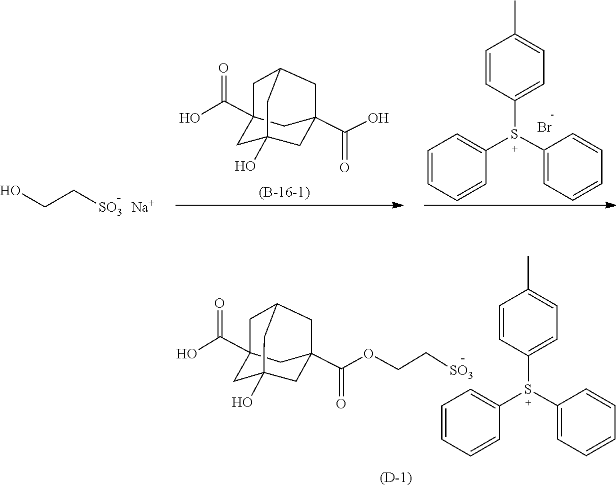

To a reaction vessel were added 20.0 mmol of sodium isethionate, 20.0 mmol of the compound (B-16-1), 20.0 mmol of dicyclohexylcarbodiimide and 50 g of dichloromethane, and the mixture was stirred at room temperature for 10 hours. Thereafter, 50 g of water was added to dilute the mixture, and then 20.0 mmol of 4-methylphenyldiphenylsulfonium bromide was added, and the mixture was vigorously stirred at room temperature for 3 hours. Thereafter, dichloromethane was added, followed by extraction, and then the organic layer was separated. The resulting organic layer was dried over sodium sulfate, the solvent was then distilled off, and the residue was purified by column chromatography, affording a compound (D-1) represented by the formula (D-1) in a good yield.

### Example D2

### (Synthesis of Compound (D-2))

Acid diffusion controlling agent represented by formula (D-2) below was synthesized in the same manner as in Example D1 except that the raw materials and the precursor were appropriately changed.

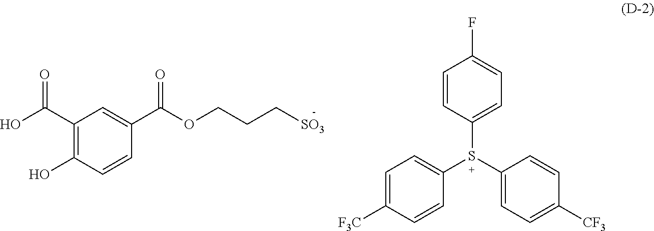

### Example D3

### (Synthesis of Compound (D-3))

A compound (D-3) was synthesized according to a synthesis scheme below.

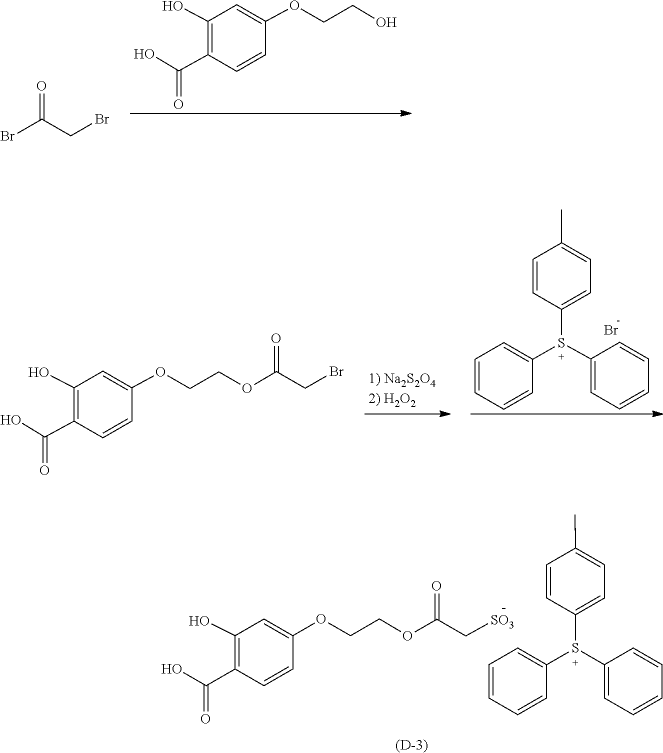

To a reaction vessel were added 20.0 mmol of bromoacetyl bromide, 20.0 mmol of 4-(2-hydroxyethoxy) salicylic acid, 20.0 mmol of triethylamine and 50 g of acetonitrile, and the mixture was stirred at room temperature for 10 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, the solvent was distilled off, and the residue was purified by column chromatography, affording an ester form in medium yield.

A mixed liquid of acetonitrile and water (1:1 (mass ratio)) was added to the ester form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. To the sodium sulfonate salt compound was added 20.0 mmol of 4-methylphenyldiphenylsulfonium bromide, and a mixed liquid of water and dichloromethane (1:3 (mass ratio)) was added thereto, forming a 0.5 M solution. The solution was vigorously stirred at room temperature for 3 hours. Thereafter, dichloromethane was added thereto, followed by extraction, and then the organic layer was separated. The resulting organic layer was dried over sodium sulfate, the solvent was then distilled off, and the residue was purified by column chromatography, affording a compound (D-3) represented by the formula (D-3) in a good yield.

### Example D4

### (Synthesis of Compound (D-4))

Acid diffusion controlling agent represented by formula (D-4) below was synthesized in the same manner as in Example D3 except that the raw materials and the precursor were appropriately changed.

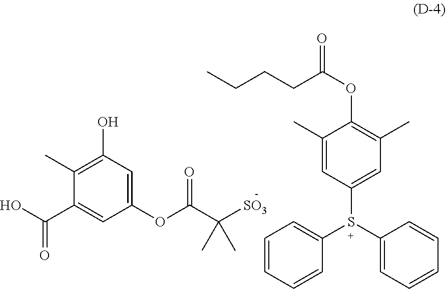

[Acid Diffusion Controlling Agent Other than Acid Diffusion Controlling Agents (D-1) to (D-4)]

d-1 todb-8: Compounds represented by formulas (d-1) to (d-8) below (hereinafter, may be described as “compound (d-1)” to “compound (d-8)”, respectively).

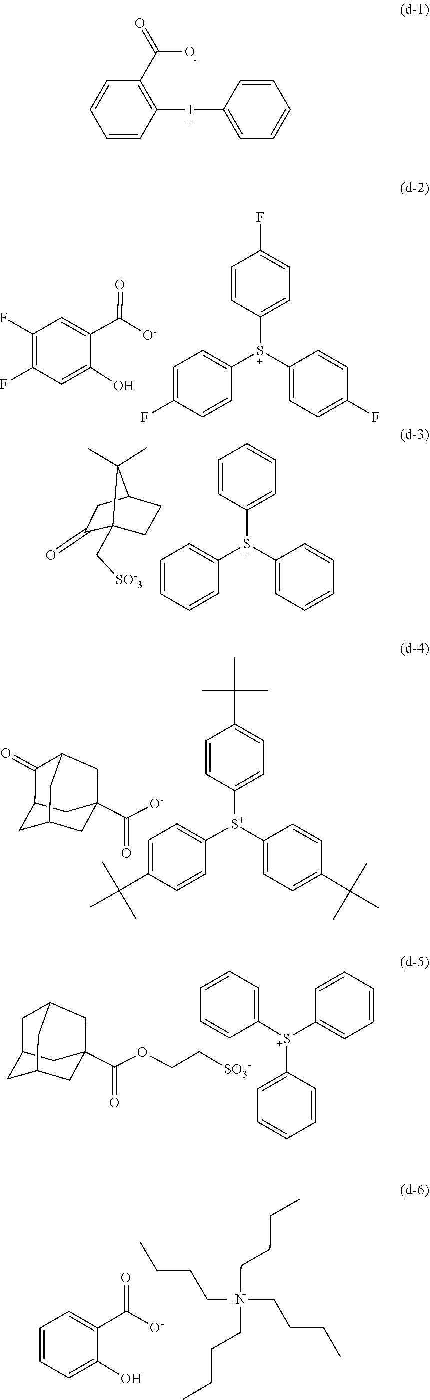

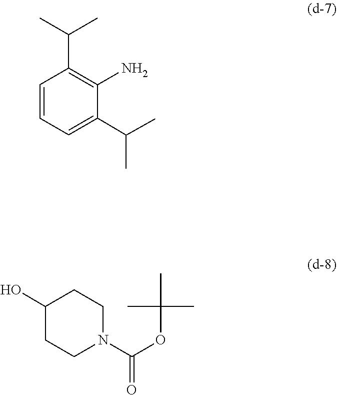

### [Solvent [E]]

E-1: propylene glycol monomethyl ether acetate
E-2: propylene glycol monomethyl ether
E-3: Y-butyrolactone

### Preparation of Positive Radiation-Sensitive Composition for ArF Immersion Exposure

### Example 1

100 parts by mass of (A-1) as the polymer [A], 10.0 parts by mass of (B-1) as the onium salt compound (1) [B] (radiation-sensitive acid generator), 6.0 parts by mass of (d-1) as the acid diffusion controlling agent [D], 5.0 parts by mass (solid content) of (F-1) as the high fluorine-content polymer [F], and 3, 400 parts by mass of a mixed solvent of (E-1)/(E-2)/(E-3) as the solvent [E] were mixed, and the mixture was filtered through a membrane filter having a pore size of 0.2 μm to prepare a radiation-sensitive composition (J-1).

### Examples 2 to 49 and Comparative Examples 1 to 6

Radiation-sensitive compositions (J-2) to (J-49) and (CJ-1) to (CJ-6) were prepared in the same manner as in Example 1 except that the components of the types and the contents shown in Table 4 below were used.

TABLE 4

| | | | | Onium salt | | Acid diffusion | | High fluorine- | | | |
|---|---|---|---|---|---|---|---|---|---|---|---|
| | | Polymer [A] | | compound [B] | | controlling agent [D] | | content polymer [F] | | Solvent [E] | |
| | Radiation- | | Content | | Content | | Content | | Content | | Content |
| | sensitive | | (parts | | (parts | | (parts | | (parts | | (parts |
| | composition | Type | by mass) | Type | by mass) | Type | by mass) | Type | by mass) | Type | by mass) |
| Example 1 | J-1 | A-1 | 100 | B-1 | 10.0 | d-1 | 6.0 | F-1 | 5.0 | E-1/E-2/E-3 | 2240/960/200 |
| Example 2 | J-2 | A-2 | 100 | B-1 | 10.0 | d-1 | 6.0 | F-1 | 5.0 | E-1/E-2/E-3 | 2240/960/200 |
| Example 3 | J-3 | A-3 | 100 | B-1 | 10.0 | d-1 | 6.0 | F-1 | 5.0 | E-1/E-2/E-3 | 2240/960/200 |
| Example 4 | J-4 | A-4 | 100 | B-1 | 10.0 | d-1 | 6.0 | F-1 | 5.0 | E-1/E-2/E-3 | 2240/960/200 |
| Example 5 | J-5 | A-5 | 100 | B-1 | 10.0 | d-1 | 6.0 | F-1 | 5.0 | E-1/E-2/E-3 | 2240/960/200 |
| Example 6 | J-6 | A-6 | 100 | B-1 | 10.0 | d-1 | 6.0 | F-1 | 5.0 | E-1/E-2/E-3 | 2240/960/200 |
| Example 7 | J-7 | A-7 | 100 | B-1 | 10.0 | d-1 | 6.0 | F-1 | 5.0 | E-1/E-2/E-3 | 2240/960/200 |
| Example 8 | J-8 | A-8 | 100 | B-1 | 10.0 | d-1 | 6.0 | F-1 | 5.0 | E-1/E-2/E-3 | 2240/960/200 |
| Example 9 | J-9 | A-9 | 100 | B-1 | 10.0 | d-1 | 6.0 | F-1 | 5.0 | E-1/E-2/E-3 | 2240/960/200 |
| Example 10 | J-10 | A-10 | 100 | B-1 | 10.0 | d-1 | 6.0 | F-1 | 5.0 | E-1/E-2/E-3 | 2240/960/200 |
| Example 11 | J-11 | A-11 | 100 | B-1 | 10.0 | d-1 | 6.0 | F-1 | 5.0 | E-1/E-2/E-3 | 2240/960/200 |
| Example 12 | J-12 | A-1 | 100 | B-1 | 10.0 | d-2 | 6.0 | F-1 | 5.0 | E-1/E-2/E-3 | 2240/960/200 |
| Example 13 | J-13 | A-1 | 100 | B-1 | 10.0 | d-3 | 6.0 | F-1 | 5.0 | E-1/E-2/E-3 | 2240/960/200 |
| Example 14 | J-14 | A-1 | 100 | B-1 | 10.0 | d-4 | 6.0 | F-1 | 5.0 | E-1/E-2/E-3 | 2240/960/200 |
| Example 15 | J-15 | A-1 | 100 | B-1 | 10.0 | d-5 | 6.0 | F-1 | 5.0 | E-1/E-2/E-3 | 2240/960/200 |
| Example 16 | J-16 | A-1 | 100 | B-1 | 10.0 | d-6 | 6.0 | F-1 | 5.0 | E-1/E-2/E-3 | 2240/960/200 |
| Example 17 | J-17 | A-1 | 100 | B-1 | 10.0 | d-7 | 6.0 | F-1 | 5.0 | E-1/E-2/E-3 | 2240/960/200 |
| Example 18 | J-18 | A-1 | 100 | B-1 | 10.0 | d-8 | 6.0 | F-1 | 5.0 | E-1/E-2/E-3 | 2240/960/200 |
| Example 19 | J-19 | A-1 | 100 | B-2 | 10.0 | d-1 | 6.0 | F-1 | 5.0 | E-1/E-2/E-3 | 2240/960/200 |
| Example 20 | J-20 | A-1 | 100 | B-3 | 10.0 | d-1 | 6.0 | F-1 | 5.0 | E-1/E-2/E-3 | 2240/960/200 |
| Example 21 | J-21 | A-1 | 100 | B-4 | 10.0 | d-1 | 6.0 | F-1 | 5.0 | E-1/E-2/E-3 | 2240/960/200 |
| Example 22 | J-22 | A-1 | 100 | B-5 | 10.0 | d-1 | 6.0 | F-1 | 5.0 | E-1/E-2/E-3 | 2240/960/200 |
| Example 23 | J-23 | A-1 | 100 | B-6 | 10.0 | d-1 | 6.0 | F-1 | 5.0 | E-1/E-2/E-3 | 2240/960/200 |
| Example 24 | J-24 | A-1 | 100 | B-7 | 10.0 | d-1 | 6.0 | F-1 | 5.0 | E-1/E-2/E-3 | 2240/960/200 |
| Example 25 | J-25 | A-1 | 100 | B-8 | 10.0 | d-1 | 6.0 | F-1 | 5.0 | E-1/E-2/E-3 | 2240/960/200 |
| Example 26 | J-26 | A-1 | 100 | B-9 | 10.0 | d-1 | 6.0 | F-1 | 5.0 | E-1/E-2/E-3 | 2240/960/200 |
| Example 27 | J-27 | A-1 | 100 | B-10 | 10.0 | d-1 | 6.0 | F-1 | 5.0 | E-1/E-2/E-3 | 2240/960/200 |
| Example 28 | J-28 | A-1 | 100 | B-11 | 10.0 | d-1 | 6.0 | F-1 | 5.0 | E-1/E-2/E-3 | 2240/960/200 |
| Example 29 | J-29 | A-1 | 100 | B-12 | 10.0 | d-1 | 6.0 | F-1 | 5.0 | E-1/E-2/E-3 | 2240/960/200 |
| Example 30 | J-30 | A-1 | 100 | B-13 | 10.0 | d-1 | 6.0 | F-1 | 5.0 | E-1/E-2/E-3 | 2240/960/200 |
| Example 31 | J-31 | A-1 | 100 | B-14 | 10.0 | d-1 | 6.0 | F-1 | 5.0 | E-1/E-2/E-3 | 2240/960/200 |
| Example 32 | J-32 | A-1 | 100 | B-15 | 10.0 | d-1 | 6.0 | F-1 | 5.0 | E-1/E-2/E-3 | 2240/960/200 |
| Example 33 | J-33 | A-1 | 100 | B-16 | 10.0 | d-1 | 6.0 | F-1 | 5.0 | E-1/E-2/E-3 | 2240/960/200 |
| Example 34 | J-34 | A-1 | 100 | B-17 | 10.0 | d-1 | 6.0 | F-1 | 5.0 | E-1/E-2/E-3 | 2240/960/200 |
| Example 35 | J-35 | A-1 | 100 | B-18 | 10.0 | d-1 | 6.0 | F-1 | 5.0 | E-1/E-2/E-3 | 2240/960/200 |
| Example 36 | J-36 | A-1 | 100 | B-19 | 10.0 | d-1 | 6.0 | F-1 | 5.0 | E-1/E-2/E-3 | 2240/960/200 |
| Example 37 | J-37 | A-1 | 100 | B-20 | 10.0 | d-1 | 6.0 | F-1 | 5.0 | E-1/E-2/E-3 | 2240/960/200 |
| Example 38 | J-38 | A-1 | 100 | B-1/b-1 | 5.0/5.0 | d-1 | 6.0 | F-1 | 5.0 | E-1/E-2/E-3 | 2240/960/200 |
| Example 39 | J-39 | A-1 | 100 | B-1/b-2 | 5.0/5.0 | d-1 | 6.0 | F-1 | 5.0 | E-1/E-2/E-3 | 2240/960/200 |
| Example 40 | J-40 | A-1 | 100 | B-1/b-3 | 5.0/5.0 | d-1 | 6.0 | F-1 | 5.0 | E-1/E-2/E-3 | 2240/960/200 |
| Example 41 | J-41 | A-1 | 100 | B-1 | 5.0 | d-1 | 6.0 | F-1 | 5.0 | E-1/E-2/E-3 | 2240/960/200 |
| Example 42 | J-42 | A-1 | 100 | B-1 | 20.0 | d-1 | 6.0 | F-1 | 5.0 | E-1/E-2/E-3 | 2240/960/200 |
| Example 43 | J-43 | A-1 | 100 | B-1 | 10.0 | d-1 | 6.0 | F-2 | 5.0 | E-1/E-2/E-3 | 2240/960/200 |
| Example 44 | J-44 | A-1 | 100 | B-1 | 10.0 | d-1 | 6.0 | F-3 | 5.0 | E-1/E-2/E-3 | 2240/960/200 |
| Example 45 | J-45 | A-1 | 100 | B-1 | 10.0 | d-1 | 6.0 | F-4 | 5.0 | E-1/E-2/E-3 | 2240/960/200 |
| Example 46 | J-46 | A-1 | 100 | b-1 | 10.0 | D-1 | 6.0 | F-1 | 5.0 | E-1/E-2/E-3 | 2240/960/200 |
| Example 47 | J-47 | A-1 | 100 | b-1 | 10.0 | D-2 | 6.0 | F-1 | 5.0 | E-1/E-2/E-3 | 2240/960/200 |
| Example 48 | J-48 | A-1 | 100 | b-1 | 10.0 | D-3 | 6.0 | F-1 | 5.0 | E-1/E-2/E-3 | 2240/960/200 |
| Example 49 | J-49 | A-1 | 100 | b-1 | 10.0 | D-4 | 6.0 | F-1 | 5.0 | E-1/E-2/E-3 | 2240/960/200 |
| Comparative | CJ-1 | A-1 | 100 | b-1 | 10.0 | d-1 | 6.0 | F-1 | 5.0 | E-1/E-2/E-3 | 2240/960/200 |
| Example 1 | | | | | | | | | | | |
| Comparative | CJ-2 | A-1 | 100 | b-4 | 10.0 | d-1 | 6.0 | F-1 | 5.0 | E-1/E-2/E-3 | 2240/960/200 |
| Example 2 | | | | | | | | | | | |
| Comparative | CJ-3 | A-1 | 100 | b-5 | 10.0 | d-1 | 6.0 | F-1 | 5.0 | E-1/E-2/E-3 | 2240/960/200 |
| Example 3 | | | | | | | | | | | |
| Comparative | CJ-4 | A-1 | 100 | b-6 | 10.0 | d-1 | 6.0 | F-1 | 5.0 | E-1/E-2/E-3 | 2240/960/200 |
| Example 4 | | | | | | | | | | | |
| Comparative | CJ-5 | A-1 | 100 | b-7 | 10.0 | d-1 | 6.0 | F-1 | 5.0 | E-1/E-2/E-3 | 2240/960/200 |
| Example 5 | | | | | | | | | | | |
| Comparative | CJ-6 | A-1 | 100 | b-8 | 10.0 | d-1 | 6.0 | F-1 | 5.0 | E-1/E-2/E-3 | 2240/960/200 |
| Example 6 | | | | | | | | | | | |

### <Formation of Resist Pattern Using Positive Radiation-Sensitive Composition for ArF Immersion Exposure>

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 100 nm. The positive radiation-sensitive composition for ArF immersion exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 110 nm. Next, the resist film was exposed through a 60 nm line-and-space mask pattern using an ArF excimer laser immersion exposure apparatus (“TWINSCAN XT-1900i” manufactured by ASML) with NA of 1.35 under an optical condition of Dipole (0=0.9/0.7). After the exposure, PEB (post exposure baking) was performed at 100° C. for 60 seconds. Thereafter, the resist film was developed with an alkali with use of a 2.38% by mass aqueous TMAH solution as an alkaline developer, followed by washing with water and further drying to form a positive resist pattern (60 nm line-and-space pattern).

### <Evaluation>

The resist pattern formed using the positive radiation-sensitive composition for ArF immersion exposure was evaluated on sensitivity, LWR, pattern rectangularity, EL and number of development defects in accordance with the following methods. The results are shown in the following Table 5. A scanning electron microscope (“CG-5000” manufactured by Hitachi High-Tech Corporation) was used for measuring the length of the resist pattern. The results are shown in the following Table 5.

### [Sensitivity]

An exposure dose at which a 60 nm line-and-space pattern was formed in the aforementioned resist pattern formation using each of the positive radiation-sensitive compositions for ArF immersion exposure was defined as an optimum exposure dose, and this optimum exposure dose was defined as sensitivity (mJ/cm²). The sensitivity was evaluated to be “good” in a case of being 30 mJ/cm² or less, and “poor” in a case of exceeding 30 mJ/cm².

### [LWR]

A 60 nm line-and-space resist pattern was formed by irradiation with the optimum exposure dose obtained in the evaluation of the sensitivity. The formed resist pattern was observed from above the pattern with use of the scanning electron microscope. The variation in the line width was measured at a total of 500 points. The 3 sigma value was obtained from the distribution of the measurement values, and defined as LWR performance (nm). The smaller the value of the LWR is, the smaller the roughness of the line is, which is better. The LWR was evaluated to be “good” in a case of being 2.5 nm or less, and “poor” in a case of exceeding 2.5 nm.

### [Pattern Rectangularity]

The 60 nm line-and-space resist pattern formed by irradiation with the optimum exposure amount obtained in the evaluation of the sensitivity was observed using the scanning electron microscope, and the sectional shape of the line-and-space pattern was evaluated. The rectangularity of the resist pattern was evaluated as “A” (extremely good) when the ratio of the length of the lower side to the length of the upper side in the sectional shape was 1 or more and 1.05 or less, “B” (good) when the ratio was more than 1.05 and 1.10 or less, and “C” (poor) when the ratio was more than 1.10.

### [Number of Development Defects]

The resist films were exposed with the optimum exposure dose to form a 60-nm line-and-space pattern and used as wafers for defect inspection. The number of defects on this wafer for defect inspection was measured using a defect inspection device (“KLA 2810” of KLA-Tencor Corporation). The defects having a diameter of 50 μm or less were determined to be derived from the resist film, and the number of the defects were calculated. When the number of defects determined to be derived from the resist film was 30 or less, the number of defects after development was evaluated as “good”, and when more than 30, evaluated as “poor”.

### [EL (Exposure Latitude)]

In the range of the exposure dose including the optimum exposure dose, resist patterns were formed by changing the exposure dose every 1 mJ/cm², and the line width of each resist pattern was measured using the scanning electron microscope. From the obtained relationship between the line width and the exposure dose, the exposure dose E(66) at which the line width was 66 nm and the exposure dose E(54) at which the line width was 54 nm were determined, and the exposure latitude (%) was calculated from the equation: exposure latitude (EL)=(E(54)−E(66))×100/(optimum exposure dose). The larger the value of the exposure latitude, the smaller the fluctuation in dimension among the patterns obtained when the exposure dose fluctuates, and the higher the yield at the time of manufacturing a device. When being 10% or more, the EL was evaluated as “good”, and when being less than 108, the EL was evaluated as “poor”.

TABLE 5

| | | | | | Number of | |
|---|---|---|---|---|---|---|
| | Radiation- | | | | development | |
| | sensitive | Sensitivity | LWR | Pattern | defects | EL |
| | composition | (mJ/cm²) | (nm) | rectangularity | (defects) | (%) |
| Example 1 | J-1 | 20 | 2.0 | A | 5 | 12.7 |
| Example 2 | J-2 | 21 | 2.1 | A | 2 | 14.7 |
| Example 3 | J-3 | 23 | 2.2 | A | 4 | 11.5 |
| Example 4 | J-4 | 24 | 2.1 | A | 4 | 15.2 |
| Example 5 | J-5 | 22 | 1.9 | A | 6 | 11.9 |
| Example 6 | J-6 | 25 | 2.0 | A | 2 | 11.4 |
| Example 7 | J-7 | 22 | 2.1 | A | 5 | 14.6 |
| Example 8 | J-8 | 21 | 2.0 | A | 0 | 14.7 |
| Example 9 | J-9 | 25 | 1.9 | A | 4 | 13.0 |
| Example 10 | J-10 | 22 | 1.8 | A | 2 | 11.4 |
| Example 11 | J-11 | 21 | 2.2 | A | 5 | 11.9 |
| Example 12 | J-12 | 21 | 2.0 | A | 2 | 15.8 |
| Example 13 | J-13 | 18 | 2.3 | A | 2 | 14.8 |
| Example 14 | J-14 | 25 | 2.0 | A | 5 | 12.7 |
| Example 15 | J-15 | 18 | 2.2 | A | 6 | 14.6 |
| Example 16 | J-16 | 26 | 2.3 | A | 7 | 11.3 |
| Example 17 | J-17 | 26 | 2.3 | A | 2 | 12.9 |
| Example 18 | J-18 | 27 | 2.3 | A | 3 | 11.9 |
| Example 19 | J-19 | 21 | 2.0 | A | 5 | 14.6 |
| Example 20 | J-20 | 25 | 1.8 | A | 4 | 15.2 |
| Example 21 | J-21 | 23 | 1.9 | A | 6 | 12.2 |
| Example 22 | J-22 | 21 | 1.7 | A | 4 | 11.1 |
| Example 23 | J-23 | 25 | 1.8 | A | 3 | 13.5 |
| Example 24 | J-24 | 25 | 2.0 | A | 7 | 11.6 |
| Example 25 | J-25 | 26 | 2.1 | A | 4 | 10.9 |
| Example 26 | J-26 | 27 | 1.8 | A | 0 | 10.5 |
| Example 27 | J-27 | 25 | 1.9 | A | 2 | 11.8 |
| Example 28 | J-28 | 23 | 1.8 | A | 2 | 12.4 |
| Example 29 | J-29 | 22 | 1.9 | A | 3 | 14.0 |
| Example 30 | J-30 | 23 | 2.0 | A | 2 | 11.1 |
| Example 31 | J-31 | 23 | 2.1 | A | 6 | 12.3 |
| Example 32 | J-32 | 25 | 2.0 | A | 7 | 15.0 |
| Example 33 | J-33 | 22 | 1.8 | A | 9 | 13.6 |
| Example 34 | J-34 | 23 | 1.9 | A | 7 | 14.1 |
| Example 35 | J-35 | 23 | 1.7 | A | 4 | 11.7 |
| Example 36 | J-36 | 26 | 2.0 | A | 6 | 13.0 |
| Example 37 | J-37 | 26 | 2.1 | A | 3 | 14.7 |
| Example 38 | J-38 | 27 | 1.9 | A | 12 | 14.3 |
| Example 39 | J-39 | 26 | 2.0 | A | 14 | 15.7 |
| Example 40 | J-40 | 27 | 1.8 | A | 11 | 13.1 |
| Example 41 | J-41 | 24 | 2.1 | A | 4 | 13.7 |
| Example 42 | J-42 | 18 | 2.0 | A | 6 | 11.6 |
| Example 43 | J-43 | 20 | 2.1 | A | 3 | 12.5 |
| Example 44 | J-44 | 20 | 2.0 | A | 5 | 12.7 |
| Example 45 | J-45 | 20 | 2.1 | A | 2 | 12.7 |
| Example 46 | J-46 | 18 | 2.3 | A | 12 | 12.3 |
| Example 47 | J-47 | 18 | 2.3 | A | 14 | 11.7 |
| Example 48 | J-48 | 19 | 2.4 | A | 11 | 16.0 |
| Example 49 | J-49 | 17 | 2.3 | A | 14 | 15.1 |
| Comparative | CJ-1 | 35 | 3.5 | B | 219 | 4.8 |
| Example 1 | | | | | | |
| Comparative | CJ-2 | 40 | 4.0 | C | 250 | 3.8 |
| Example 2 | | | | | | |
| Comparative | CJ-3 | 42 | 3.8 | C | 211 | 5.5 |
| Example 3 | | | | | | |
| Comparative | CJ-4 | 33 | 4.0 | B | 134 | 8.9 |
| Example 4 | | | | | | |
| Comparative | CJ-5 | 35 | 3.8 | B | 140 | 7.8 |
| Example 5 | | | | | | |
| Comparative | CJ-6 | 32 | 3.3 | C | 101 | 9.2 |
| Example 6 | | | | | | |

As is apparent from the results in Table 5, the radiation-sensitive compositions of Examples were good in sensitivity, LWR, pattern rectangularity, EL and development defect performance when used for ArF immersion exposure, whereas the radiation-sensitive compositions of Comparative Examples were inferior in the characteristics to those of Examples. Therefore, when the radiation-sensitive compositions of Examples are used for ArF immersion exposure, resist patterns having high sensitivity, good LWR, EL, and pattern rectangularity and having less development defects can be formed.

### Preparation of Positive Radiation-Sensitive Composition for Extreme Ultraviolet (EUV) Exposure

### Example 50

100 parts by mass of (A-12) as the polymer [A], 30.0 parts by mass of (B-1) as the onium salt compound (1) [B] (radiation-sensitive acid generator), 20.0 parts by mass of (d-1) as the acid diffusion controlling agent [D], 5.0 parts by mass (solid content) of (F-5) as the high fluorine-content polymer [F], and 6,000 parts by mass of a mixed solvent of (E-1)/(E-2)/(E-3) as the solvent [E] were mixed, and the mixture was filtered through a membrane filter having a pore size of 0.2 μm to prepare a radiation-sensitive composition (J-50).

### Examples 51 to 65 and Comparative Examples 7 to 9

Radiation-sensitive compositions (J-51) to (J-65) and (CJ-7) to (CJ-9) were prepared in the same manner as in Example 50 except that the components of the types and the contents shown in Table 6 below were used.

TABLE 6

| | | | | Onium salt | | Acid diffusion | | High fluorine- | | | |
|---|---|---|---|---|---|---|---|---|---|---|---|
| | | Polymer [A] | | compound [B] | | controlling agent [D] | | content polymer [F] | | Solvent [E] | |
| | Radiation- | | Content | | Content | | Content | | Content | | Content |
| | sensitive | | (parts | | (parts | | (parts | | (parts | | (parts |
| | composition | Type | by mass) | Type | by mass) | Type | by mass) | Type | by mass) | Type | by mass) |
| Example 50 | J-50 | A-12 | 100 | B-1 | 30.0 | d-1 | 20.0 | F-5 | 5.0 | E-1/E-2/E-3 | 1000/4900/100 |
| Example 51 | J-51 | A-13 | 100 | B-1 | 30.0 | d-1 | 20.0 | F-5 | 5.0 | E-1/E-2/E-3 | 1000/4900/100 |
| Example 52 | J-52 | A-14 | 100 | B-1 | 30.0 | d-1 | 20.0 | F-5 | 5.0 | E-1/E-2/E-3 | 1000/4900/100 |
| Example 53 | J-53 | A-15 | 100 | B-1 | 30.0 | d-1 | 20.0 | F-5 | 5.0 | E-1/E-2/E-3 | 1000/4900/100 |
| Example 54 | J-54 | A-12 | 100 | B-1 | 30.0 | d-2 | 20.0 | F-5 | 5.0 | E-1/E-2/E-3 | 1000/4900/100 |
| Example 55 | J-55 | A-12 | 100 | B-1 | 30.0 | d-4 | 20.0 | F-5 | 5.0 | E-1/E-2/E-3 | 1000/4900/100 |
| Example 56 | J-56 | A-12 | 100 | B-3 | 30.0 | d-1 | 20.0 | F-5 | 5.0 | E-1/E-2/E-3 | 1000/4900/100 |
| Example 57 | J-57 | A-12 | 100 | B-4 | 30.0 | d-1 | 20.0 | F-5 | 5.0 | E-1/E-2/E-3 | 1000/4900/100 |
| Example 58 | J-58 | A-12 | 100 | B-5 | 30.0 | d-1 | 20.0 | F-5 | 5.0 | E-1/E-2/E-3 | 1000/4900/100 |
| Example 59 | J-59 | A-12 | 100 | B-6 | 30.0 | d-1 | 20.0 | F-5 | 5.0 | E-1/E-2/E-3 | 1000/4900/100 |
| Example 60 | J-60 | A-12 | 100 | B-11 | 30.0 | d-1 | 20.0 | F-5 | 5.0 | E-1/E-2/E-3 | 1000/4900/100 |
| Example 61 | J-61 | A-12 | 100 | B-14 | 30.0 | d-1 | 20.0 | F-5 | 5.0 | E-1/E-2/E-3 | 1000/4900/100 |
| Example 62 | J-62 | A-12 | 100 | B-18 | 30.0 | d-1 | 20.0 | F-5 | 5.0 | E-1/E-2/E-3 | 1000/4900/100 |
| Example 63 | J-63 | A-12 | 100 | B-20 | 30.0 | d-1 | 20.0 | F-5 | 5.0 | E-1/E-2/E-3 | 1000/4900/100 |
| Example 64 | J-64 | A-12 | 100 | B-1/b-1 | 15.0/15.0 | d-1 | 20.0 | F-5 | 5.0 | E-1/E-2/E-3 | 1000/4900/100 |
| Example 65 | J-65 | A-12 | 100 | B-1/b-3 | 15.0/15.0 | d-1 | 20.0 | F-5 | 5.0 | E-1/E-2/E-3 | 1000/4900/100 |
| Comparative | CJ-7 | A-12 | 100 | b-4 | 30.0 | d-1 | 20.0 | F-5 | 5.0 | E-1/E-2/E-3 | 1000/4900/100 |
| Example 7 | | | | | | | | | | | |
| Comparative | CJ-8 | A-12 | 100 | b-5 | 30.0 | d-1 | 20.0 | F-5 | 5.0 | E-1/E-2/E-3 | 1000/4900/100 |
| Example 8 | | | | | | | | | | | |
| Comparative | CJ-9 | A-12 | 100 | b-7 | 30.0 | d-1 | 20.0 | F-5 | 5.0 | E-1/E-2/E-3 | 1000/4900/100 |
| Example 9 | | | | | | | | | | | |

### <Formation of Resist Pattern Using Positive Radiation-Sensitive Composition for EUV Exposure>

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 105 nm. The positive radiation-sensitive composition for EUV exposure prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR02. After exposing, PEB was performed at 120° C. for 60 seconds. Thereafter, the resist film was developed with an alkali with use of a 2.38% by mass aqueous TMAH solution as an alkaline developer, followed by washing with water and further drying to form a positive resist pattern (25 nm line-and-space pattern).

### <Evaluation>

The resist pattern formed using the positive radiation-sensitive composition for EUV exposure was evaluated on sensitivity, LWR, EL and number of development defects in accordance with the following methods. The results are shown in the following Table 7. A scanning electron microscope (“CG-5000” manufactured by Hitachi High-Tech Corporation) was used for measuring the length of the resist pattern.

### [Sensitivity]

An exposure dose at which a 25 nm line-and-space pattern was formed in the aforementioned resist pattern formation using the positive radiation-sensitive composition for EUV exposure was defined as an optimum exposure dose, and this optimum exposure dose was defined as sensitivity (mJ/cm²). The sensitivity was evaluated to be “good” in a case of being 40 mJ/cm² or less, and “poor” in a case of exceeding 40 mJ/cm².

### [LWR]

A resist pattern was formed by adjusting a mask size so as to form a 25 nm line-and-space pattern by irradiation with the optimum exposure dose obtained in the evaluation of the sensitivity. The formed resist pattern was observed from above the pattern with use of the scanning electron microscope. The variation in the line width was measured at a total of 500 points. The 3 sigma value was obtained from the distribution of the measurement values, and defined as LWR (nm). The smaller the value of the LWR is, the smaller the wobble of the line is, which is better. The LWR was evaluated to be “good” in a case of being 3.0 nm or less, and “poor” in a case of exceeding 3.0 nm.

### [Number of Development Defects]

The resist films were exposed with the optimum exposure dose to form a 25-nm line-and-space pattern and used as wafers for defect inspection. The number of defects on this wafer for defect inspection was measured using a defect inspection device (“KLA 2810” of KLA-Tencor Corporation). The defects having a diameter of 50 μm or less were determined to be derived from the resist film, and the number of the defects were calculated. When the number of defects determined to be derived from the resist film was 50 or less, the number of defects after development was evaluated as “good”, and when more than 50, evaluated as “poor”.

### [EL (Exposure Latitude)]

In the range of the exposure dose including the optimum exposure dose, resist patterns were formed by changing the exposure dose every 1 mJ/cm², and the line width of each resist pattern was measured using the scanning electron microscope. From the obtained relationship between the line width and the exposure dose, the exposure dose E(28) at which the line width was 28 nm and the exposure dose E(22) at which the line width was 22 nm were determined, and the exposure latitude (%) was calculated from the equation: exposure latitude (EL)=(E(22)−E(28))×100/(optimum exposure dose). The larger the value of the exposure latitude, the smaller the fluctuation in dimension among the patterns obtained when the exposure dose fluctuates, and the higher the yield at the time of manufacturing a device. When being 7% or more, the EL was evaluated as “good”, and when being less than 78, the EL was evaluated as “poor”.

TABLE 7

| | | | | Number of | |
|---|---|---|---|---|---|
| | Radiation- | | | development | |
| | sensitive | Sensitivity | LWR | defects | EL |
| | composition | (mJ/cm²) | (nm) | (defects) | (%) |
| Example 50 | J-50 | 33 | 2.5 | 3 | 11.2 |
| Example 51 | J-51 | 34 | 2.3 | 4 | 10.2 |
| Example 52 | J-52 | 33 | 2.4 | 2 | 11.4 |
| Example 53 | J-53 | 32 | 2.5 | 6 | 13.9 |
| Example 54 | J-54 | 30 | 2.4 | 2 | 10.0 |
| Example 55 | J-55 | 35 | 2.6 | 5 | 11.0 |
| Example 56 | J-56 | 34 | 2.1 | 2 | 10.2 |
| Example 57 | J-57 | 32 | 2.4 | 5 | 13.4 |
| Example 58 | J-58 | 32 | 2.1 | 2 | 12.8 |
| Example 59 | J-59 | 34 | 2.4 | 1 | 13.8 |
| Example 60 | J-60 | 31 | 2.1 | 1 | 10.3 |
| Example 61 | J-61 | 33 | 2.2 | 2 | 10.3 |
| Example 62 | J-62 | 32 | 2.3 | 3 | 10.3 |
| Example 63 | J-63 | 33 | 2.3 | 5 | 10.4 |
| Example 64 | J-64 | 35 | 2.6 | 3 | 12.1 |
| Example 65 | J-65 | 32 | 2.1 | 7 | 10.4 |
| Comparative | CJ-7 | 50 | 4.1 | 191 | 2.3 |
| Example 7 | | | | | |
| Comparative | CJ-8 | 48 | 4.4 | 186 | 4.5 |
| Example 8 | | | | | |
| Comparative | CJ-9 | 45 | 4.2 | 153 | 5.1 |
| Example 9 | | | | | |

As is apparent from the results in Table 7, the radiation-sensitive compositions of Examples were good in sensitivity, LWR, EL and development defect performance when used for EUV exposure, whereas the radiation-sensitive compositions of Comparative Examples were inferior in the characteristics to those of Examples.

### Preparation of Negative Radiation-Sensitive Composition for ArF Exposure, and Formation and Evaluation of Resist Pattern Using this Composition

### Example 66

100 parts by mass of (A-8) as the polymer [A], 8.0 parts by mass of (B-1) as the onium salt compound (1) [B] (radiation-sensitive acid generator), 7.0 parts by mass of (d-2) as the acid diffusion controlling agent [D], 2.0 parts by mass (solid content) of (F-4) as the high fluorine-content polymer [F], and 3,230 parts by mass of a mixed solvent of (E-1)/(E-2)/(E-3) (mass ratio: 2,240/960/30) as the solvent [E] were mixed, and the mixture was filtered through a membrane filter having a pore size of 0.2 μm to prepare a radiation-sensitive composition (J-66).

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 100 nm. The negative radiation-sensitive composition for ArF exposure (J-66) prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 90 nm. Next, this resist film was exposed through a mask pattern having a hole of 50 nm and a pitch of 100 nm using an ArF excimer laser immersion exposure apparatus (“TWINSCAN XT-1900i” manufactured by ASML) with NA of 1.35 under an optical condition of Annular (σ=0.8/0.6). After the exposure, PEB (post exposure baking) was performed at 100° C. for 60 seconds. Thereafter, the resist film was developed with an organic solvent using n-butyl acetate as an organic solvent developer, and dried to form a negative resist pattern (contact hole pattern with hole of 50 nm and pitch of 100 nm).

The resist pattern using the negative radiation-sensitive composition for ArF exposure was evaluated on sensitivity in the same manner as in the evaluation of the resist pattern using the positive radiation-sensitive composition for ArF exposure. In addition, CDU and pattern circularity were evaluated in accordance with the following methods.

### [CDU]

Contact holes with a 50 nm hole and a 100 nm pitch were formed by irradiation with an optimum exposure dose determined in the evaluation of sensitivity. The formed resist pattern was observed from above the pattern with use of the scanning electron microscope. The variation of the diameters of the contact holes was measured at 500 points in total. The 3 sigma value was determined from the distribution of the measurement values, and defined as CDU (nm). The smaller the value of the CDU is, the smaller the roughness of the contact holes is, which is better. When the value was less than 3.5 nm, the CDU was evaluated to be “good”, and when the value was 3.5 nm or more, the CDU was evaluated to be “poor”.

### [Pattern Circularity]

The contact holes with a 50 nm hole and a 100 nm pitch formed by irradiation with the optimum exposure dose determined in the evaluation of sensitivity were observed in plan view using the scanning electron microscope, and the size in the longitudinal direction and the size in the lateral direction were measured. When the ratio of the size in the longitudinal direction to the size in the lateral direction was 0.95 or more and less than 1.05, the pattern circularity was evaluated as “A” (extremely good), when the ratio was 0.90 or more and less than 0.95, or 1.05 or more and less than 1.10, the pattern circularity was evaluated as “B” (good), and when the ratio was less than 0.90, or 1.10 or more, the pattern circularity was evaluated as “C” (poor).

As a result, the radiation-sensitive composition of Example 66 had good sensitivity, CDU, and pattern circularity even when a negative resist pattern was formed by ArF exposure.

[Preparation of Negative Radiation-Sensitive Composition for EUV Exposure, and Formation and Evaluation of Resist Pattern Using this Composition]

### Example 67

100 parts by mass of (A-13) as the polymer [A], 30.0 parts by mass of (B-5) as the onium salt compound (1) [B] (radiation-sensitive acid generator), 15.0 parts by mass of (d-4) as the acid diffusion controlling agent [D], 2.0 parts by mass (solid content) of (F-5) as the high fluorine-content polymer [F], and 6,000 parts by mass of a mixed solvent of (E-1)/(E-2)/(E-3) (mass ratio: 1,000/4, 900/100) as the solvent [E] were mixed, and the mixture was filtered through a membrane filter having a pore size of 0.2 μm to prepare a radiation-sensitive composition (J-67).

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 105 nm. The negative radiation-sensitive composition for EUV exposure (J-67) prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR15. After exposing, PEB was performed at 120° C. for 60 seconds. Thereafter, the resist film was developed with an organic solvent using n-butyl acetate as an organic solvent developer, and dried to form a negative resist pattern (contact hole pattern with hole of 20 nm and pitch of 40 nm).

The resist pattern formed using the negative radiation-sensitive composition for EUV exposure was evaluated in the same manner as the resist pattern formed using the negative radiation-sensitive composition for ArF exposure. As a result, the radiation-sensitive composition of Example 67 had good sensitivity, CDU, and pattern circularity even when a negative resist pattern was formed by EUV exposure.

According to the radiation-sensitive composition, the method for forming a pattern and the radiation-sensitive acid generator described above, a resist pattern having good sensitivity to exposure light and being superior in LWR, pattern rectangularity, development defect performance, EL, CDU, and pattern circularity can be formed. Therefore, these can be suitably used for a machining process and the like of a semiconductor device in which micronization is expected to further progress in the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
an onium salt compound represented by formula (1);
a polymer comprising a structural unit which comprises an acid-dissociable group; and
a solvent, 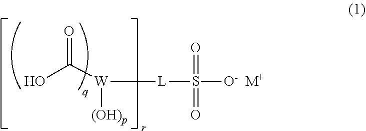
wherein, in the formula (1), W is an organic group having 3 to 40 carbon atoms and having at least one cyclic structure; L is a (r+1)-valent linking group, and r is an integer of 1 to 3; when r is 1, p and q are each independently an integer of 1 to 3, and when r is 2 or 3, each of a plurality of p's and a plurality of q's are each independently an integer of 0 to 3, provided that when r is 2 or 3, at least one of a plurality of p's is 1 or more and at least one of a plurality of q's is 1 or more; M⁺ is a monovalent onium cation.

2. The radiation-sensitive composition according to claim 1, wherein a partial structure “—W(OH)ₚ(COOH)_{q}” in the formula (1) comprises one or more groups selected from the group consisting of groups represented by formula (W-1), formula (W-2), formula (W-3), formula (W-4) and formula (W-5), 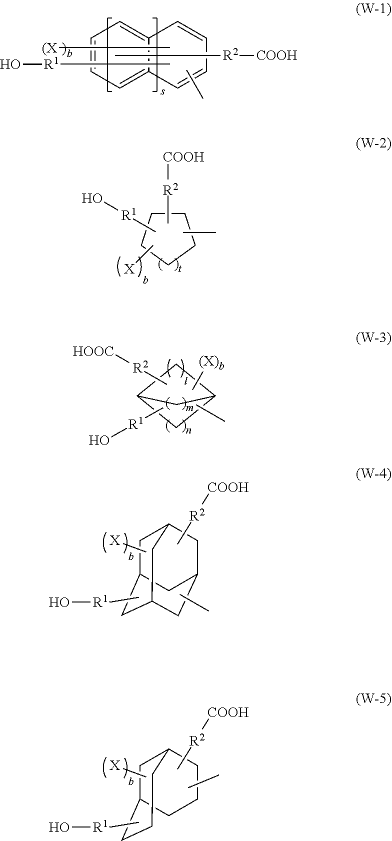
wherein, s is an integer of 0 to 2 and t is an integer of 1 to 3; 1, m, and n are each independently an integer of 1 to 6; X is a hydrogen atom, an organic group having 1 to 12 carbon atoms, a cyano group, a hydroxy group, or a halogen atom; b is an integer of 1 to 10; when b is 2 or more, a plurality of X's are the same or different from each other; R¹ and R² are the same or different from each other, and are each independently a single bond or a divalent organic group.

3. The radiation-sensitive composition according to claim 1, wherein a partial structure “—W(OH)ₚ(COOH)_{q}” in the formula (1) comprises:
one or more groups selected from the group consisting of groups represented by formula (W-6), formula (W-7), formula (W-8), and formula (W-9); and
one or more groups selected from the group consisting of groups represented by formula (W-10), formula (W-11), formula (W-12), and formula (W-13), 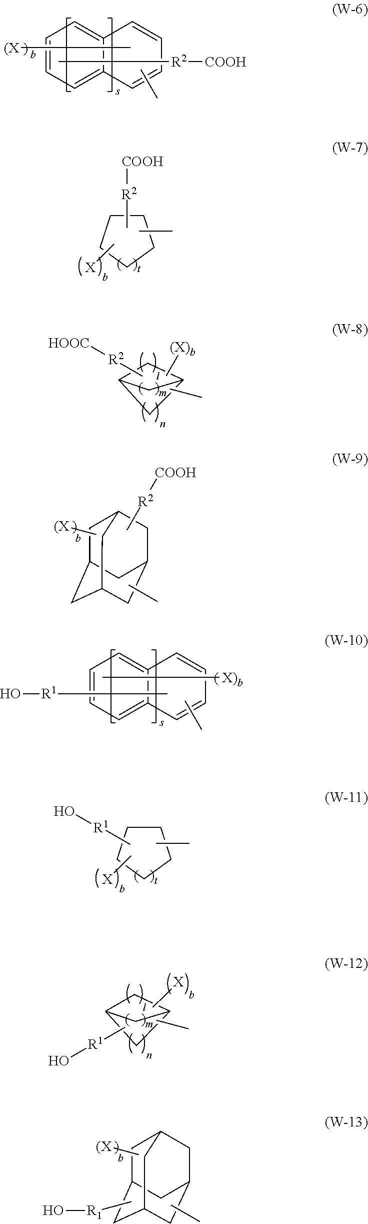
wherein, s is an integer of 0 to 2 and t is an integer of 1 to 3; 1, m, and n are each independently an integer of 1 to 6; X is a hydrogen atom, an organic group having 1 to 12 carbon atoms, a cyano group, a hydroxy group, or a halogen atom; b is an integer of 1 to 10; when b is 2 or more, a plurality of X's are the same or different from each other; R¹ and R² are the same or different from each other, and are each independently a single bond or a divalent organic group.

4. The radiation-sensitive composition according to claim 1, wherein the L has one or more bonding groups selected from the group consisting of an ether bond, an amide bond, an ester bond, and an acetal bond.

5. The radiation-sensitive composition according to claim 4, wherein the L is at least one structure selected from the group consisting of structures represented by formula (L-1), formula (L-2), formula (L-3), formula (L-4), and formula (L-5), 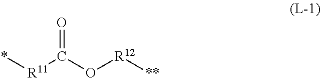
wherein, in the formula (L-1), R¹¹ is a single bond or a substituted or unsubstituted divalent hydrocarbon group having 1 to 12 carbon atoms; R¹² is a substituted or unsubstituted divalent hydrocarbon group having 1 to 12 carbon atoms; * is a bond bonding to W in the formula (1), and ** is a bond bonding to S of SO₃⁻ in the formula (1), 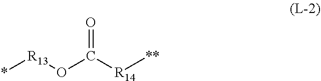
wherein, in the formula (L-2), R¹³ is a single bond or a substituted or unsubstituted divalent hydrocarbon group having 1 to 12 carbon atoms; R¹⁴ is a substituted or unsubstituted divalent hydrocarbon group having 1 to 12 carbon atoms; * is a bond bonding to W in the formula (1), and ** is a bond bonding to S of SO₃⁻ in the formula (1), 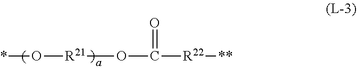
wherein, in the formula (L-3), R²¹ and R²² are the same or different from each other and are each independently a substituted or unsubstituted divalent hydrocarbon group having 1 to 12 carbon atoms, and a is an integer of 1 to 3; * is a bond bonding to W in the formula (1), and ** is a bond bonding to S of SO₃⁻ in the formula (1), 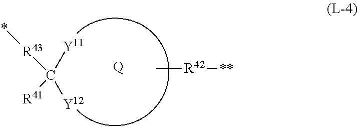
wherein, in the formula (L-4), Y¹¹ and Y¹² are each independently an oxygen atom or a sulfur atom; R⁴¹ is a hydrogen atom, a substituted or unsubstituted monovalent hydrocarbon group having 1 to 10 carbon atoms, or a monovalent organic group represented by —X¹—Y—X² and having 1 to 12 carbon atoms, wherein X¹ is a single bond or a divalent hydrocarbon group having 1 to 11 carbon atoms, Y is —O—, —CO—, —COO—, —OCO—, —OCOO—, —NHCO—, or —CONH—, and X² is a substituted or unsubstituted monovalent hydrocarbon group having 1 to 12 carbon atoms; R⁴² is a single bond or a substituted or unsubstituted divalent hydrocarbon group having 1 to 10 carbon atoms; R⁴³ is a single bond or a divalent organic group; Q is a cyclic (thio) acetal structure that forms a monocyclic ring or a condensed ring together with Y¹¹, Y¹², and the carbon atom to which Y¹¹ and Y¹² are bonded; * is a bond bonding to W in the formula (1), and ** is a bond bonding to S of SO₃⁻ in the formula (1), 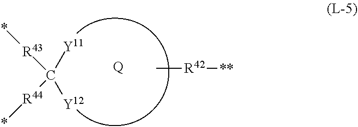
wherein, in the formula (L-5), Y¹¹, Y¹², R⁴², R⁴³, and Q are each defined as in the formula (L-4); R⁴⁴ is a single bond or a divalent organic group; * is a bond bonding to W in the formula (1), and ** is a bond bonding to S of SO₃⁻ in the formula (1).

6. The radiation-sensitive composition according to claim 1, wherein the L comprises a cyclic structure, and the cyclic structure of the L and the cyclic structure of W form a spiro ring structure.

7. The radiation-sensitive composition according to claim 1, wherein a carboxy group and a hydroxy group bonded to W in the formula (1) are bonded directly to the same cyclic structure or different cyclic structures.

8. The radiation-sensitive composition according to claim 1, wherein the structural unit having an acid-dissociable group is represented by formula (2), 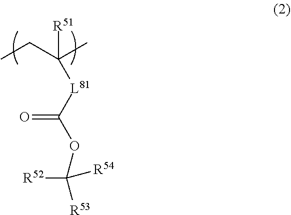
wherein, in the formula (2), R⁵¹ is a hydrogen atom, a fluorine atom, a methyl group, or a trifluoromethyl group; R⁵² is a substituted or unsubstituted monovalent hydrocarbon group having 1 to 20 carbon atoms; R⁵³ and R⁵⁴ each independently are a monovalent chain hydrocarbon group having 1 to 10 carbon atoms or a monovalent alicyclic hydrocarbon group having 3 to 20 carbon atoms, or R⁵³ and R⁵⁴ taken together represent a divalent alicyclic group having 3 to 20 carbon atoms together with the carbon atom to which R⁵³ and R⁵⁴ are bonded; L⁸¹ is a single bond or a divalent organic group.

9. A method for forming a pattern, comprising:
applying the radiation-sensitive composition according to claim 1 directly or indirectly to a substrate to form a resist film;
exposing the resist film to light; and
developing the exposed resist film.

10. The method according to claim 9, wherein developing comprises developing the exposed resist film with an alkaline developer.

11. The method according to claim 9, wherein exposing comprises exposing the resist film to an ArF excimer laser or an extreme ultraviolet ray.

12. A radiation-sensitive acid generator represented by formula (1), 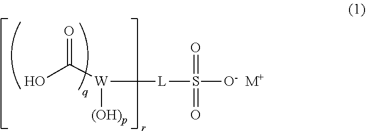
wherein, in the formula (1), W is an organic group having 3 to 40 carbon atoms and having at least one cyclic structure; L is a (r+1)-valent linking group, and r is an integer of 1 to 3; when r is 1, p and q are each independently an integer of 1 to 3, and when r is 2 or 3, each of a plurality of p's and a plurality of q's are each independently an integer of 0 to 3, provided that when r is 2 or 3, at least one of a plurality of p's is 1 or more and at least one of a plurality of q's is 1 or more; M⁺ is a monovalent onium cation.

13. The radiation-sensitive acid generator according to claim 12, wherein a partial structure “—W(OH)ₚ(COOH)_{q}” in the formula (1) comprises one or more groups selected from the group consisting of groups represented by formula (W-1), formula (W-2), formula (W-3), formula (W-4), and formula (W-5), 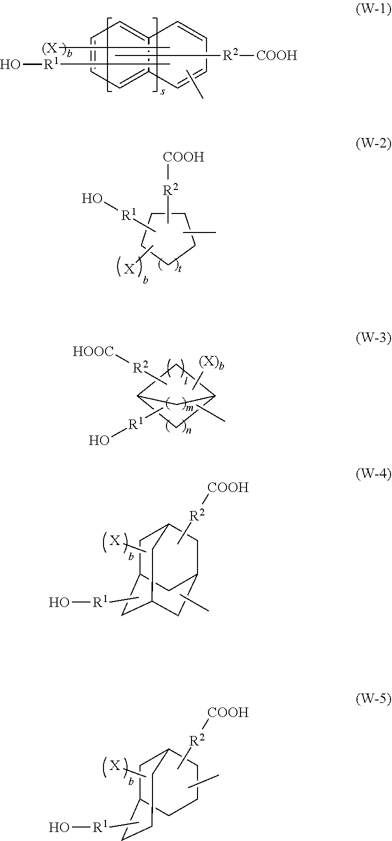
wherein, s is an integer of 0 to 2 and t is an integer of 1 to 3; 1, m, and n are each independently an integer of 1 to 6; X is a hydrogen atom, an organic group having 1 to 12 carbon atoms, a cyano group, a hydroxy group, or a halogen atom; b is an integer of 1 to 10; when b is 2 or more, a plurality of X's are the same or different from each other; R¹ and R² are the same or different from each other, and are each independently a single bond or a divalent organic group.
